SEQUENCE LISTING (65 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 80 aa, Homo sapiens
ITCPPPMSVEHADIWVKSYSLYSRERYICNSGFKRKAGTSSLTECVLNKATNVAHWTTPSLKCIRDPALVHQRPAPPSTV

SEQ ID NO: 3 - protein, 7 aa, Artificial Sequence
  anti CTLA-4 light chain CDR
QSVGSSY

SEQ ID NO: 4 - protein, 9 aa, Artificial Sequence
  anti CTLA-4 light chain CDR
QQYGSSPWT

SEQ ID NO: 5 - protein, 8 aa, Artificial Sequence
  anti CTLA-4 heavy chain CDR
GFTFSSYT

SEQ ID NO: 6 - protein, 8 aa, Artificial Sequence
  anti CTLA-4 heavy chain CDR
ISYDGNNK

SEQ ID NO: 7 - protein, 11 aa, Artificial Sequence
  anti CTLA-4 heavy chain CDR
ARTGWLGPFDY

SEQ ID NO: 8 - protein, 448 aa, Artificial Sequence
  Ipilimumab heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 215 aa, Artificial Sequence
  anti CTLA-4 light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, Artificial Sequence
  anti CTLA-4 heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 448 aa, Artificial Sequence
  anti CTLA-4 heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 118 aa, Artificial Sequence
  anti CTLA-4 heavy chain sequence
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 13 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 20 aa, Artificial Sequence
  glycine-serine linker
GGGGSGGGGSGGGGSGGGGS

SEQ ID NO: 16 - protein, 678 aa, Artificial Sequence
  anti CTLA-4 heavy chain (hole constant region) - IL-15Ra sushi domain - IL15
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGSGGGGSGGGGSITCPPPMSVEHADIWVKSYSLYSRERYICNSGFKRKAGTSSLTECVLNKATNVAHWTTPSLKCIRDPALVHQRPAPPSTVGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - DNA, 645 bp, Artificial Sequence
  anti CTLA-4 light chain
gagattgtgctgacccagagcccaggtacactgtcactgtccccaggcgagagggctactctgtcttgccgggcaagccagtctgtgggtagctcttacctggcctggtaccagcagaagccaggacaggctccacgactgctgatctacggagcattctcaagagccaccgggattcctgaccgcttcagtggctcaggctccgggacagacttcaccctgacaatctcccgactggagccagaagacttcgccgtgtactattgccagcagtatgggtccagcccctggacctttggtcagggcaccaaggtcgagatcaaacgtacagtggccgctccctccgtcttcatttttccccctagcgacgaacagctgaagtctggaaccgctagtgtggtctgtctgctgaacaatttctaccctcgcgaagcaaaggtgcagtggaaagtcgataacgccctgcagagcggcaattctcaggagagtgtgactgaacaggactcaaaggattccacctatagcctgtctagtacactgactctgtccaaagctgattacgagaagcacaaagtgtatgcatgtgaagtcactcatcaggggctgtcttcaccagtcaccaagtccttcaatcgtggagaatgc

SEQ ID NO: 18 - DNA, 2034 bp, Artificial Sequence
  anti CTLA-4 heavy chain (hole constant region) - IL-15Ra sushi domain - IL-15
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgcgtcgacaaagggcccctccgtgtttcctctggctccaagctctaagagcacctctggaggaacagccgctctgggatgtctggtgaaggattacttccctgagccagtgaccgtgagctggaactctggcgccctgacctctggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtcatgtgccgtgaagggcttctacccatctgacatcgctgtggagtgggagtccaatggccagcccgagaacaattataagaccacaccacccgtgctggactccgatggctcattcttcctggtgtccaagctgaccgtggacaagtctagatggcagcagggcaacgtgttctcctgctccgtgatgcacgaggccctgcacaatcactacacccagaagtccctgtctctgtcccctggaaaaggcggaggcggaggatctggtggtggtggatctggcggcggaggctctattacatgccctcctccaatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgcatcagggaccccgctctggttcatcagaggcctgctcctccatctaccgttggtggcggaggtagcggtggtggcggtagcggaggcggtggttctggcggaggcggttctaattgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaaaacctgatcatcctggccaacaactccctgtcctccaacggcaacgtgaccgagtctggctgcaaagagtgcgaggaactggaagagaagaacatcaaagagttcctccagtccttcgtgcacatcgtgcagatgttcatcaacaccagc

SEQ ID NO: 19 - DNA, 1344 bp, Artificial Sequence
  anti CTLA-4 heavy chain
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacccttcgattattggggccagggcaccctggtgacagtgtcttccgcgtcgacaaagggcccctccgtgtttcctctggctccaagctctaagagcacctctggaggaacagccgctctgggatgtctggtgaaggattacttccctgagccagtgaccgtgagctggaactctggcgccctgacctctggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtggtgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacaatcattatacacagaaatctctgtccctgagcccaggcaag

SEQ ID NO: 20 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 21 - DNA, 342 bp, Homo sapiens
aattgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaaaacctgatcatcctggccaacaactccctgtcctccaacggcaacgtgaccgagtctggctgcaaagagtgcgaggaactggaagagaagaacatcaaagagttcctccagtccttcgtgcacatcgtgcagatgttcatcaacaccagc

SEQ ID NO: 22 - DNA, 240 bp, Homo sapiens
attacatgccctcctccaatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgcatcagggaccccgctctggttcatcagaggcctgctcctccatctaccgtt

SEQ ID NO: 23 - protein, 4 aa, Artificial Sequence
  glycine-serine linker
GGGS

SEQ ID NO: 24 - protein, 5 aa, Artificial Sequence
  glycine-serine linker
GGGGS

SEQ ID NO: 25 - protein, 10 aa, Artificial Sequence
  glycine-serine linker
GGGGSGGGGS

SEQ ID NO: 26 - protein, 15 aa, Artificial Sequence
  glycine-serine linker
GGGGSGGGGSGGGGS

SEQ ID NO: 27 - DNA, 48 bp, Artificial Sequence
  glycine-serine linker
ggcggaggcggaggatctggtggtggtggatctggcggcggaggctct

SEQ ID NO: 28 - DNA, 354 bp, Artificial Sequence
  heavy chain variable region
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttcc

SEQ ID NO: 29 - DNA, 990 bp, Artificial Sequence
  heavy chain constant region
gcgtcgacaaagggcccctccgtgtttcctctggctccaagctctaagagcacctctggaggaacagccgctctgggatgtctggtgaaggattacttccctgagccagtgaccgtgagctggaactctggcgccctgacctctggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtcatgtgccgtgaagggcttctacccatctgacatcgctgtggagtgggagtccaatggccagcccgagaacaattataagaccacaccacccgtgctggactccgatggctcattcttcctggtgtccaagctgaccgtggacaagtctagatggcagcagggcaacgtgttctcctgctccgtgatgcacgaggccctgcacaatcactacacccagaagtccctgtctctgtcccctggaaaa

SEQ ID NO: 30 - DNA, 990 bp, Artificial Sequence
  heavy chain constant region
gcgtcgacaaagggcccctccgtgtttcctctggctccaagctctaagagcacctctggaggaacagccgctctgggatgtctggtgaaggattacttccctgagccagtgaccgtgagctggaactctggcgccctgacctctggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtggtgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacaatcattatacacagaaatctctgtccctgagcccaggcaag

SEQ ID NO: 31 - DNA, 1344 bp, Artificial Sequence
  anti CTLA-4 heavy chain
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgcgtcgacaaagggcccctccgtgtttcctctggctccaagctctaagagcacctctggaggaacagccgctctgggatgtctggtgaaggattacttccctgagccagtgaccgtgagctggaactctggcgccctgacctctggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtcatgtgccgtgaagggcttctacccatctgacatcgctgtggagtgggagtccaatggccagcccgagaacaattataagaccacaccacccgtgctggactccgatggctcattcttcctggtgtccaagctgaccgtggacaagtctagatggcagcagggcaacgtgttctcctgctccgtgatgcacgaggccctgcacaatcactacacccagaagtccctgtctctgtcccctggaaaa

SEQ ID NO: 32 - DNA, 60 bp, Artificial Sequence
  glycine-serine linker
ggtggcggaggtagcggtggtggcggtagcggaggcggtggttctggcggaggcggttct

SEQ ID NO: 33 - protein, 6 aa, Artificial Sequence
  anti CTLA-4 light chain CDR
QSINSY

SEQ ID NO: 34 - protein, 9 aa, Artificial Sequence
  anti CTLA-4 light chain CDR
QQYYSTPFT

SEQ ID NO: 35 - protein, 7 aa, Artificial Sequence
  anti CTLA-4 heavy chain CDR
GFTFSSG

SEQ ID NO: 36 - protein, 10 aa, Artificial Sequence
  anti CTLA-4 heavy chain CDR
AVIWYDGSNK

SEQ ID NO: 37 - protein, 18 aa, Artificial Sequence
  anti CTLA-4 heavy chain CDR
ARDPRGATLYYYYYGMDV

SEQ ID NO: 38 - DNA, 1716 bp, Artificial Sequence
  anti CTLA-4 heavy chain - IL-15
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcctccaccaagggcccaagcgtgtttccactggctcccagctctaagtccaccagcggaggaacagccgctctgggctgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatccaatacaaaggtggacaagagagtggagcccaagagctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgctaagacaaagcctagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggccaagggccagcctagggagccacaggtgtacaccctgcctccatgccgggaggagatgacaaagaaccaggtgtccctgtggtgtctggtgaagggcttctatcctagcgacatcgctgtggagtgggagtctaatggccagccagagaacaattacaagaccacaccccctgtgctggactccgatggcagcttctttctgtattctaagctgaccgtggataagtcccggtggcagcagggcaacgtgttttcttgttccgtgatgcatgaggccctgcacgctcattacacacagaagagcctgtctctgtccccaggcaagggaggaggaggatctggaggaggaggatccaactgggtgaatgtgatcagcgacctgaagaagatcgaggatctgatccagtctatgcacatcgacgccaccctgtatacagagagcgatgtgcatccctcttgcaaggtgaccgctatgaagtgtttcctgctggagctgcaggtcatctctctggagtccggcgacgcctccatccacgataccgtggagaatctgatcatcctggctaacaattccctgagctctaacggcaatgtgacagagagcggctgcaaggagtgtgaggagctggaggaaaagaacatcaaggagttcctgcagtcttttgtgcatatcgtgcagatgtttatcaatacctcc

SEQ ID NO: 39 - protein, 572 aa, Artificial Sequence
  anti CTLA-4 heavy chain - IL-15
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHAHYTQKSLSLSPGKGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 40 - DNA, 1344 bp, Artificial Sequence
  anti CTLA-4 heavy chain
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgccagcaccaagggcccatccgtgttcccactggctcccagctctaagagcacctctggaggaacagccgctctgggatgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatctaatacaaaggtggacaagagagtggagcccaagtcctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgccaagaccaagccaagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggctaagggccagcctagggagccacaggtgtgcaccctgcctccatcccgggaggagatgacaaagaaccaggtgtccctgagctgtgccgtgaagggcttctaccctagcgacatcgctgtggagtgggagtctaatggccagccagagaacaattataagaccacaccccctgtgctggactccgatggcagcttctttctggtgtctaagctgaccgtggataagtcccggtggcagcagggcaacgtgttctcctgttccgtgatgcatgaggccctgcacgctcattacacacagaagagcctgtctctgtcccctggcaag

SEQ ID NO: 41 - protein, 448 aa, Artificial Sequence
  anti CTLA-4 heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHAHYTQKSLSLSPGK

SEQ ID NO: 42 - protein, 678 aa, Artificial Sequence
  BS3 heavy chain (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGGSGGGGSGGGGSITCPPPMSVEHADIWVKSYSLYSRERYICNSGFKRKAGTSSLTECVLNKATNVAHWTTPSLKCIRDPALVHQRPAPPSTVGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - DNA, 2034 bp, Artificial Sequence
  BS3 heavy chain (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgcctcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacatgtctggtgaagggcttctacccatctgacatcgctgtggagtgggagtccaatggccagcccgagaacaattataagaccacaccacccgtgctggactccgatggctcattcttcctgacctccaagctgaccgtggacaagtctagatggcagcagggcaacgtgttctcctgctccgtgatgcacgaggccctgcacaatcactacacccagaagtccctgtctctgtcccctggaaaaggcggaggcggaggatctggtggtggtggatctggcggcggaggctctattacatgccctcctccaatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgcatcagggaccccgctctggttcatcagaggcctgctcctccatctaccgttggtggcggaggtagcggtggtggcggtagcggaggcggtggttctggcggaggcggttctaattgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaaaacctgatcatcctggccaacaactccctgtcctccaacggcaacgtgaccgagtctggctgcaaagagtgcgaggaactggaagagaagaacatcaaagagttcctccagtccttcgtgcacatcgtgcagatgttcatcaacaccagc

SEQ ID NO: 44 - protein, 534 aa, Artificial Sequence
  Ab-IL-15Su (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLTSKLTVDKSRWQQGNVFSCSVMHEALHAHYTQKSLSLSPGKGGGGSGGGGSITCPPPMSVEHADIWVKSYSLYSRERYICNSGFKRKAGTSSLTECVLNKATNVAHWTTPSLKCIRDPALVHQRPAP

SEQ ID NO: 45 - DNA, 1602 bp, Artificial Sequence
  Ab-IL-15su (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgccagcaccaagggcccatccgtgttcccactggctcccagctctaagagcacctctggaggaacagccgctctgggatgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatctaatacaaaggtggacaagagagtggagcccaagtcctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgccaagaccaagccaagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggctaagggccagcctagggagccacaggtgtgcaccctgcctccatcccgggaggagatgacaaagaaccaggtgtccctgagctgtgccgtgaagggcttctaccctagcgacatcgctgtggagtgggagtctaatggccagccagagaacaattataagaccacaccccctgtgctggactccgatggcagcttctttctggtgtctaagctgaccgtggataagtcccggtggcagcagggcaacgtgttctcctgttccgtgatgcatgaggccctgcacgctcattacacacagaagagcctgtctctgtcccctggcaagggaggaggaggaagcggaggaggaggatctatcacctgtccaccccctatgtccgtggagcatgccgacatctgggtgaagtcctacagcctgtattccagggagcggtatatctgcaatagcggcttcaagaggaaggctggcaccagctctctgacagagtgcgtgctgaacaaggccaccaatgtggctcattggaccacacccagcctgaagtgtatccgggatcctgccctggtgcaccagcgccccgccccc

SEQ ID NO: 46 - protein, 572 aa, Artificial Sequence; identical to SEQ ID NO: 39
  G2 heavy chain construct (knob)

SEQ ID NO: 47 - DNA, 1716 bp, Artificial Sequence
  G2 construct heavy chain (knob)
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcctccaccaagggcccaagcgtgtttccactggctcccagctctaagtccaccagcggaggaacagccgctctgggctgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatccaatacaaaggtggacaagagagtggagcccaagagctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgctaagacaaagcctagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggccaagggccagcctagggagccacaggtgtacaccctgcctccatgccgggaggagatgacaaagaaccaggtgtccctgtggtgtctggtgaagggcttctatcctagcgacatcgctgtggagtgggagtctaatggccagccagagaacaattacaagaccacaccccctgtgctggactccgatggcagcttctttctgtattctaagctgaccgtggataagtcccggtggcagcagggcaacgtgttttcttgttccgtgatgcatgaggccctgcacgctcattacacacagaagagcctgtctctgtccccaggcaagggaggaggaggatctggaggaggaggatctaactgggtgaatgtgatcagcgacctgaagaagatcgaggatctgatccagtctatgcacatcgacgccaccctgtatacagagagcgatgtgcatccctcttgcaaggtgaccgctatgaagtgtttcctgctggagctgcaggtcatctctctggagtccggcgacgcctccatccacgataccgtggagaatctgatcatcctggctaacaattccctgagctctaacggcaatgtgacagagagcggctgcaaggagtgtgaggagctggaggaaaagaacatcaaggagttcctgcagtcttttgtgcatatcgtgcagatgtttatcaatacctcc

SEQ ID NO: 48 - protein, 538 aa, Artificial Sequence
  G2 construct heavy chain (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHAHYTQKSLSLSPGKGGGGSGGGGSITCPPPMSVEHADIWVKSYSLYSRERYICNSGFKRKAGTSSLTECVLNKATNVAHWTTPSLKCIRDPALVHQRPAPPSTV

SEQ ID NO: 49 - DNA, 1614 bp, Artificial Sequence
  G2 construct heavy chain (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgccagcaccaagggcccatccgtgttcccactggctcccagctctaagagcacctctggaggaacagccgctctgggatgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatctaatacaaaggtggacaagagagtggagcccaagtcctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgccaagaccaagccaagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggctaagggccagcctagggagccacaggtgtgcaccctgcctccatcccgggaggagatgacaaagaaccaggtgtccctgagctgtgccgtgaagggcttctaccctagcgacatcgctgtggagtgggagtctaatggccagccagagaacaattataagaccacaccccctgtgctggactccgatggcagcttctttctggtgtctaagctgaccgtggataagtcccggtggcagcagggcaacgtgttctcctgttccgtgatgcatgaggccctgcacgctcattacacacagaagagcctgtctctgtcccctggcaagggaggaggaggatctggaggaggaggaagcatcacctgtccaccccctatgtccgtggagcatgccgacatctgggtgaagtcctacagcctgtattccagggagcggtatatctgcaatagcggcttcaagaggaaggctggcaccagctctctgacagagtgcgtgctgaacaaggccaccaatgtggctcattggaccacacccagcctgaagtgtatccgggatcctgccctggtgcaccagcgccccgccccccccagcaccgtg

SEQ ID NO: 50 - protein, 576 aa, Artificial Sequence
  G3 construct heavy chain (knob)
QVQLVESGGGVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHAHYTQKSLSLSPGKGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 51 - DNA, 1731 bp, Artificial Sequence
  G3 construct heavy chain (knob)
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcctccaccaagggcccaagcgtgtttccactggctcccagctctaagtccaccagcggaggaacagccgctctgggctgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatccaatacaaaggtggacaagagagtggagcccaagagctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgctaagacaaagcctagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggccaagggccagcctagggagccacaggtgtacaccctgcctccaagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacgctcattatacacagaaatctctgtccctgagcccaggcaagggaggaggaggaagtggaggaggcggatctggaggaggaggatccaactgggtgaatgtgatcagcgacctgaagaagatcgaggatctgatccagtctatgcacatcgacgccaccctgtatacagagagcgatgtgcatccctcttgcaaggtgaccgctatgaagtgtttcctgctggagctgcaggtcatctctctggagtccggcgacgcctccatccacgataccgtggagaatctgatcatcctggctaacaattccctgagctctaacggcaatgtgacagagagcggctgcaaggagtgtgaggagctggaggaaaagaacatcaaggagttcctgcagtcttttgtgcatatcgtgcagatgtttatcaatacctcc

SEQ ID NO: 52 - protein, 544 aa, Artificial Sequence
  G3 construct heavy chain (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHAHYTQKSLSLSPGKGGGGGSGGGGSGGGGSITCPPPMSVEHADIWVKSYSLYSRERYICNSGFKRKAGTSSLTECVLNKATNVAHWTTPSLKCIRDPALVHQRPAPPSTV

SEQ ID NO: 53 - DNA, 1632 bp, Artificial Sequence
  G3 construct heavy chain (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgccagcaccaagggcccatccgtgttcccactggctcccagctctaagagcacctctggaggaacagccgctctgggatgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatctaatacaaaggtggacaagagagtggagcccaagtcctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgccaagaccaagccaagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggctaagggccagcctagggagccacaggtgtacaccctgcctccaagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgacctccaagctgaccgtggataagagcaggtggcagcagggcaacgtgttttcctgcagcgtgatgcacgaggccctgcacgctcattatacacagaaatctctgtccctgagcccaggcaagggcggaggagggggatctggaggaggaggaagcggaggaggaggatctatcacctgtccaccccctatgtccgtggagcatgccgacatctgggtgaagtcctacagcctgtattccagggagcggtatatctgcaatagcggcttcaagaggaaggctggcaccagctctctgacagagtgcgtgctgaacaaggccaccaatgtggctcattggaccacacccagcctgaagtgtatccgggatcctgccctggttcaccaacgccccgccccccccagcaccgtg

SEQ ID NO: 54 - protein, 582 aa, Artificial sequence
  G4 construct heavy chain (knob)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHAHYTQKSLSLSPGKGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 55 - DNA, 1746 bp, Artificial Sequence
  G4 construct heavy chain (knob)
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcctccaccaagggcccaagcgtgtttccactggctcccagctctaagtccaccagcggaggaacagccgctctgggctgtcttgtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatccaatacaaaggtggacaagagagtggagcccaagagctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgctaagacaaagcctagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggccaagggccagcctagggagccacaggtgtacaccctgcctccaagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacgctcattatacacagaaatctctgtccctgagcccaggcaagggtggaggcggttctggaggaggcggaagtggaggtggaggatctggaggaggaggatccaactgggtgaatgtgatcagcgacctgaagaagatcgaggatctgatccagtctatgcacatcgacgccaccctgtatacagagagcgatgtgcatccctcttgcaaggtgaccgctatgaagtgtttcctgctggagctgcaggtcatctctctggagtccggcgacgcctccatccacgataccgtggagaatctgatcatcctggctaacaattccctgagctctaacggcaatgtgacagagagcggctgcaaggagtgtgaggagctggaggaaaagaacatcaaggagttcctgcagtcttttgtgcatatcgtgcagatgtttatcaatacctcc

SEQ ID NO: 56 - protein, 548 aa, Artificial Sequence
  G4 construct heavy chain (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPAVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHAHYTQKSLSLSPGKGGGGSGGGGSGGGGSGGGGSITCPPPMSVEHADIWVKSYSLYSRERYICNSGFKRKAGTSSLTECVLNKATNVAHWTTPSLKCIRDPALVHQRPAPPSTV

SEQ ID NO: 57 - DNA, 1644 bp, Artificial Sequence
  G4 construct heavy chain (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgccagcaccaagggcccatccgtgttcccactggctcccagctctaagagcacctctggaggaacagccgctctgggatgtctggtgaaggattacttcccagagcccgtgacagtgagctggaactctggcgccctgacctctggcgtgcatacatttccagctgtgctgcagtccagcggcctgtactccctgtcttccgtggtgaccgtgcccagctcttccctgggcacccagacatatatctgcaacgtgaatcacaagccatctaatacaaaggtggacaagagagtggagcccaagtcctgtgataagacccatacatgccccccttgtcctgctccagagttcctgggaggaccagccgtgttcctgtttccacccaagcctaaggacaccctgatgatcagccgcaccccagaggtgacatgcgtggtggtggacgtgtctcacgaggatcctgaggtgaagtttaactggtacgtggatggcgtggaggtgcataatgccaagaccaagccaagggaggagcagtacaactccacctatcgggtggtgagcgtgctgacagtgctgcaccaggactggctgaacggcaaggagtataagtgtaaggtgtccaataaggccctgcccgctcctatcgagaagaccatcagcaaggctaagggccagcctagggagccacaggtgtacaccctgcctccaagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgacctccaagctgaccgtggataagagcaggtggcagcagggcaacgtgttttcctgcagcgtgatgcacgaggccctgcacgctcattatacacagaaatctctgtccctgagcccaggcaagggaggcggaggatctggaggaggcggttctggaggaggaggaagcggaggaggaggatctatcacctgtccaccccctatgtccgtggagcatgccgacatctgggtgaagtcctacagcctgtattccagggagcggtatatctgcaatagcggcttcaagaggaaggctggcaccagctctctgacagagtgcgtgctgaacaaggccaccaatgtggctcattggaccacacccagcctgaagtgtatccgggatcctgccctggtgcaccagcgccccgccccacctagcaccgtg

SEQ ID NO: 58 - protein, 592 aa, Artificial Sequence
  G6 construct heavy chain (knob)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - DNA, 1776 bp, Artificial Sequence
  G6 construct heavy chain (knob)
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcctcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacatgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggctctgcacaaccactacacccagaagtccctgtctctgtcccctggaaaaggcggcggaggatctggcggaggtggaagcggaggcggaggaagcggtggcggcggtagtggcggtggtggttcaggtggtggtggctctaattgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaaaacctgatcatcctggccaacaactccctgtcctccaacggcaacgtgaccgagtctggctgcaaagagtgcgaggaactggaagagaagaacatcaaagagttcctccagtccttcgtgcacatcgtgcagatgttcatcaacaccagc

SEQ ID NO: 60 - protein, 560 aa, Artificial Sequence
  G6 construct heavy chain (hole)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGGSGGGGSGGGGSGGGGGSGGGGSGGGGSITCPPPMSVEHADIWVKSYSLYSRERYICNSGFKRKAGTSSLTECVLNKATNVAHWTTPSLKCIRDPALVHQRPAPPSTV

SEQ ID NO: 61 - DNA, 1681 bp, Artificial Sequence
  G6 construct heavy chain (hole)
caggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgcctcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacatgtctggtgaagggcttctacccatctgacatcgctgtggagtgggagtccaatggccagcccgagaacaattataagaccacaccacccgtgctggactccgatggcagcttctttctgacctccaagctgaccgtggataagagcaggtggcagcagggcaacgtgttttcctgcagcgtgatgcacgaggctctgcacaaccactacacccagaagtccctgtctctgtcccctggaaaaggcggaggcggaggatctggtggtggtggatctggcggcggaggaagcggcggaggcggcggaagtggtggaggaggttctggcggaggtggcagcattacatgccctccacctatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgcatcagggaccccgctctggttcatcagaggcctgctcctccatccaccgtgt

SEQ ID NO: 62 - protein, 448 aa, Artificial Sequence; identical to SEQ ID NO: 10
  BS2 construct heavy chain (knob)

SEQ ID NO: 63 - DNA, 1344 bp, Artificial Sequence
  BS2 construct heavy chain (knob)
caggtgcagctggtggagagcggaggaggagtggtgcagccaggcaggagcctgaggctgtcttgcgctgcttccggcttcaccttttccagctacacaatgcactgggtgaggcaggctcctggcaagggactggagtgggtgaccttcatctcttatgacggcaacaataaatactatgctgattccgtgaagggcaggttcaccatcagccgcgacaactctaagaatacactgtacctgcagatgaactctctgagagccgaggacaccgctatctactattgcgcccgcacaggatggctgggacctttcgattattggggccagggcaccctggtgacagtgtcttccgcgtcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgtggtgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacaatcattatacacagaaatctctgtccctgagcccaggcaag

SEQ ID NO: 64 - protein, 677 aa, Artificial Sequence
  BS2 construct heavy chain (hole)
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSITCPPPMSVEHADIWVKSYSLYSRERYICNSGFKRKAGTSSLTECVLNKATNVAHWTTPSLKCIRDPALVHQRPAPPSTVGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 65 - DNA, 2031 bp, Artificial Sequence
  BS2 construct heavy chain (hole)
aactgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaaaacctgatcatcctggccaacaactccctgtcctccaacggcaacgtgaccgagtctggctgcaaagagtgcgaggaactggaagagaagaacatcaaagagttcctccagtccttcgtgcacatcgtgcagatgttcatcaacacctctggcggcggaggatctggcggaggtggaagcggaggcggaggaagcggtggcggcggatctatcacctgtcctccacctatgtccgtggaacacgccgacatctgggtcaagtcctacagcctgtactccagagagcggtacatctgcaactccggcttcaagagaaaggcgggcacctctagcctgaccgagtgcgtgctgaacaaggccaccaatgtggcccactggaccacacctagcctgaagtgcatcagggaccccgctctggttcatcagaggcctgctcctccatctacagtcggaggcggaggatctggtggtggtggatctggcggcggaggtagccaggtgcagctggtggagagcggaggaggagtggtgcagccaggcaggtctctgaggctgtcctgcgctgctagcggcttcaccttttccagctacacaatgcactgggtgaggcaggctcctggcaagggcctggagtgggtgaccttcatctcctatgacggcaacaataaatactatgctgatagcgtgaagggcaggttcaccatctctcgcgacaactccaagaatacactgtacctgcagatgaactctctgagagccgaggacaccgctatctactattgcgctaggacaggatggctgggacctttcgattattggggccagggcaccctggtgacagtgtcttccgcgtcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagctgctgggaggaccttccgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacatgtctggtgaagggcttctacccatctgacatcgctgtggagtgggagtccaatggccagcccgagaacaattataagaccacaccacccgtgctggactccgatggcagcttctttctgacctccaagctgaccgtggataagagcaggtggcagcagggcaacgtgttttcctgcagcgtgatgcacgaggccctgcacaatcattatacacagaaatctctgtccctgagcccaggcaag

(19) United States
(12) Patent Application Publication
(10) Pub. No.: US 2024/0254184 A1
(43) Pub. Date: Aug. 1, 2024

(54) IL-15 FUSION PROTEINS AND METHODS OF MAKING AND USING THE SAME

(71) Applicant: Salubris Biotherapeutics, Inc., Gaithersburg, MD (US)

(72) Inventors: John LI, Reston, VA (US); Binyam Z. BEZABEH, Silver Spring, MD (US); Ming WU, Ashburn, VA (US)

(21) Appl. No.: 18/274,668

(22) PCT Filed: Feb. 4, 2022
(86) PCT No.: PCT/US2022/015271

§ 371 Date: Jul. 27, 2023

Related U.S. Application Data
(60) Provisional application No. 63/146,242, filed on Feb. 5, 2021.

(51) Int. Cl. C07K 14/54; A61K 38/20; A61K 39/00; A61K 39/395; A61P 35/00; C07K 14/715; C07K 16/28; C12N 15/70

(52) U.S. Cl. CPC C07K 14/5443; A61K 38/2086; A61K 39/3955; A61K 39/4611; A61K 39/4613; A61K 39/4631; A61K 39/4632; A61P 35/00; C07K 14/7155; C07K 16/2818; C12N 15/70; A61K 2039/505; C07K 2317/569; C07K 2317/622; C07K 2317/76; C07K 2319/30; C12N 2800/101

(57) ABSTRACT

The disclosure provides recombinant fusion proteins comprising an antigen binding domain specific for CTLA-4, an IL-15Ra sushi domain and IL-15. The disclosure further provides methods of using these recombinant fusion proteins in the treatment of cancer.

### CROSS-REFERENCE TO RELATED APPLICATIONS

This application claims priority to, and benefit of, U.S. Provisional Application No. 63/146,242, filed on Feb. 5, 2021, the contents of which are incorporated by reference in their entirety herein.

### INCORPORATION BY REFERENCE OF SEQUENCE LISTING

The contents of the text file submitted electronically herewith are incorporated herein by reference in their entirety: A computer readable format copy of the Sequence Listing (filename: SBTI-002-001WO_SeqList_ST25.txt, date recorded: Jan. 31, 2022, file size 145 kilobytes).

### BACKGROUND

Cancer is one of the leading causes of death in the developed world. In the United States alone, an estimated 1.8 million people were newly diagnosed, and over 600,000 cancer deaths occurred in 2020. In cancer, cells of the subject grow and divide abnormally, spreading into surrounding tissues. Each cancer is thought to have combination of genetic changes, which may vary between cancers that allow cancer cells to escape the body's natural controls on cellular proliferation and allow the cancer to spread. While some cancers are currently treatable, many cancers are not. The instant disclosure provides a recombinant fusion protein comprising an antigen binding domain specific to CTLA-4, an Interleukin 15 receptor subunit alpha chain (IL-15Ra) sushi domain, and Interleukin 15 (IL-15), and compositions comprising the same, for the treatment of cancer.

### SUMMARY

The instant disclosure is based on the finding that a recombinant fusion protein comprising an anti-CTLA-4 antigen binding domain, in combination with IL-15 and an IL-15Ra sushi domain, can be used to treat cancer by effectively promoting the activity and proliferation of immune cells, which respond to cancer antigens expressed by the cancer cells and mount an immune response against the cancer. Unexpectedly, fusion proteins of the disclosure comprising an anti-CTLA-4 antigen binding domain, IL-15Ra sushi domain, and IL-15 show limited induction of interferon gamma (IFNγ) in vivo, an inflammatory cytokine which is thought to negatively affect the safety and tolerability of IL-15 in clinical studies, while maintaining the ability to expand CD8 and NK cell populations.

Accordingly, the disclosure provides a recombinant fusion protein comprising: (a) an interleukin 15 (IL-15) domain; (b) an interleukin 15 receptor subunit alpha (IL-15Ra) sushi domain; and (c) a cytotoxic T-lymphocyte associated protein 4 (CTLA-4) antigen binding domain. In some embodiments, the IL-15 domain and IL-15Ra sushi domain are separated by a GGGGSGGGGSGGGGSGGGGS (SEQ ID NO: 15) linker.

In some embodiments of the recombinant fusion proteins of the disclosure, the IL-15 domain is active. In some embodiments, the IL-15Ra sushi domain increases the activity of the IL-15 domain compared to the activity of an IL-15 domain in an otherwise equivalent recombinant fusion protein lacking the IL-15Ra sushi domain.

In some embodiments of the recombinant fusion proteins of the disclosure, the IL-15 domain comprises a sequence of SEQ ID NO: 1, or a sequence having at least 80%, at least 85%, at least 90%, at least 95% or at least 99% identity thereto. In some embodiments, the IL-15 domain comprises, or consists essentially of, a sequence of SEQ ID NO: 1.

In some embodiments of the recombinant fusion proteins of the disclosure, the IL-15Ra sushi domain comprises a sequence of SEQ ID NO: 2, or a sequence having at least 80%, at least 85%, at least 90%, at least 95% or at least 99% identity thereto. In some embodiments, the IL-15Ra sushi domain comprises, or consists essentially of, a sequence of SEQ ID NO: 2.

In some embodiments of the recombinant fusion proteins of the disclosure, the CTLA-4 antigen binding domain comprises a heavy chain comprising complementarity determining region (CDR) sequences of GFTFSSYT (SEQ ID NO: 5), ISYDGNNK (SEQ ID NO: 6) and ARTGWLGPFDY (SEQ ID NO: 7). In some embodiments, the CTLA-4 antigen binding domain comprises a light chain comprising CDR sequences of QSVGSSY (SEQ ID NO: 3), GAF and QQYGSSPWT (SEQ ID NO: 4). In some embodiments, the CTLA-4 antigen binding domain comprises a single chain variable fragment (scFv), a single-domain antibody (sdAb), an antibody, or an antibody fragment.

In some embodiments of the recombinant fusion proteins of the disclosure, the CTLA-4 antigen binding domain comprises a CTLA-4 antibody. In some embodiments, the CTLA-4 antibody comprises a first heavy chain and second heavy chain. In some embodiments, the first and second heavy chains both comprise a heavy chain variable region sequence of SEQ ID NO: 12, or a sequence having at least 80%, at least 85%, at least 90%, at least 95% or at least 99% identity thereto. In some embodiments, the first and second heavy chains both comprise a heavy chain variable region sequence of SEQ ID NO: 12. In some embodiments, the first heavy chain comprises a constant region sequence of SEQ ID NO: 13, or a sequence having at least 80%, at least 85%, at least 900%, at least 95% or at least 99% identity thereto. In some embodiments, the first heavy chain comprises a constant region sequence of SEQ ID NO: 13. In some embodiments, the second heavy chain comprises a constant region sequence of SEQ ID NO: 14, or a sequence having at least 80%, at least 85%, at least 90%, at least 95% or at least 99% identity thereto. In some embodiments, the second heavy chain comprises a constant region sequence of SEQ ID NO: 14. In some embodiments, the first heavy chain comprises a sequence of SEQ ID NO: 11, or a sequence having at least 80%, at least 85%, at least 90%, at least 95% or at least 99% identity thereto. In some embodiments, the second heavy chain comprises a sequence of SEQ ID NO: 10, or a sequence having at least 80%, at least 85%, at least 90%, at least 95% or at least 99% identity thereto. In some embodiments, the first and second heavy chains preferentially form a heterodimer.

In some embodiments of the recombinant fusion proteins of the disclosure, the CTLA-4 antigen binding domain comprises a CTLA-4 antibody. In some embodiments, the CTLA-4 antibody comprises a first heavy chain and second heavy chain. In some embodiments, the second heavy chain comprises, from N to C terminus, an anti-CTLA-4 heavy chain, a first linker, an IL-15Ra sushi domain, a second linker, and an IL-15 domain. In some embodiments, the first heavy chain comprises a sequence of SEQ ID NO: 10, and the second heavy chain comprises, from N to C terminus, a heavy chain sequence of SEQ ID NO: 11, a first linker comprising a sequence of SEQ ID NO: 26, an IL-15Ra sushi domain comprising a sequence of SEQ ID NO: 2, a second linker comprising a sequence of SEQ ID NO: 15 and an IL-15 domain comprising a sequence of SEQ ID NO: 1. In some embodiments, the first heavy chain comprises a sequence of SEQ ID NO: 10 and the second heavy chain comprises a sequence of SEQ ID NO: 16. In some embodiments, the first heavy chain comprises a sequence of SEQ ID NO; 11, and the second heavy chain comprises, from N to C terminus, a heavy chain sequence of SEQ ID NO: 10, a first linker comprising a sequence of SEQ ID NO: 26, an IL-15Ra sushi domain comprising a sequence of SEQ ID NO: 2, a second linker comprising a sequence of SEQ ID NO: 15 and an IL-15 domain comprising a sequence of SEQ ID NO: 1. In some embodiments, the CTLA-4 antibody comprises a light chain sequence comprising SEQ ID NO: 9.

In some embodiments of the recombinant fusion proteins of the disclosure, the N-terminus of the IL-15Ra sushi domain is linked to the C-terminus of the first or second heavy chain. In some embodiments, the N-terminus of IL-15 domain is linked to the C-terminus of the IL-15Ra sushi domain. In some embodiments, the first or second heavy chain and the IL-15Ra domain are separated by a linker. In some embodiments, the IL-15Ra sushi domain and the IL-15 domain are separated by a linker. In some embodiments, the linker comprises a sequence of GGGS (SEQ ID NO: 23), GGGGS (SEQ ID NO: 24), GGGGSGGGGS (SEQ ID NO: 25), GGGGSGGGGSGGGGS (SEQ ID NO: 26), or GGGGSGGGGSGGGGSGGGGS (SEQ ID NO: 15).

In some embodiments of the recombinant fusion proteins of the disclosure, the first heavy chain, IL-15Ra sushi domain and IL-15 domain comprise a sequence of SEQ ID NO: 16, or a sequence having at least 80%, at least 85%, at least 90%, at least 95% or at least 99% identity thereto. In some embodiments of the recombinant fusion proteins of the disclosure, the first heavy chain, IL-15Ra sushi domain and IL-15 domain comprise a sequence of SEQ ID NO: 16.

In some embodiments of the recombinant fusion proteins of the disclosure, the CTLA-4 antibody comprises a light chain sequence comprising SEQ ID NO: 9, or a sequence having at least 80%, at least 85%, at least 90%, at least 95% or at least 99% identity thereto. In some embodiments, the CTLA-4 antibody comprises a light chain sequence comprising SEQ ID NO: 9.

The disclosure provides a recombinant fusion protein, comprising: (a) a first polypeptide comprising, from N- to C-terminus, sequences of a first CTLA-4 antibody heavy chain, an IL-15Ra sushi domain and an IL-15 domain; (b) a second polypeptide comprising a sequence of a second CTLA-4 heavy chain; and (c) two additional polypeptides comprising a sequence of a CTLA-4 antibody light chain. In some embodiments, the IL-15 domain and IL-15Ra sushi domain are separated by a GGGGSGGGGSGGGGSGGGGS (SEQ ID NO: 15) linker.

In some embodiments of the recombinant fusion proteins of the disclosure, the first and second polypeptides preferentially form a heterodimer.

In some embodiments of the recombinant fusion proteins of the disclosure, the first polypeptide comprises a sequence of SEQ ID NO: 16, the second polypeptide comprises a sequence of SEQ ID NO: 10, and the CTLA-4 antibody light chain comprises a sequence of SEQ ID NO: 9, or sequences having at least 80%, at least 85%, at least 90%, at least 95% or at least 99% identity thereto. In some embodiments of the recombinant fusion proteins of the disclosure, the first polypeptide comprises a sequence of SEQ ID NO: 16, the second polypeptide comprises a sequence of SEQ ID NO: 10, and the CTLA-4 antibody light chain comprises a sequence of SEQ ID NO: 9.

The disclosure provides polynucleotides encoding the recombinant fusion proteins of the disclosure.

The disclosure provides polynucleotides encoding the first polypeptide, the second polypeptide, or the CTLA-4 antibody light chain of the disclosure.

In some embodiments of the polynucleotides of the disclosure, the sequence encoding the CTLA-4 antibody light chain comprises a sequence of SEQ ID NO: 17, or a sequence having at least 80%, at least 85%, at least 90%, at least 95% or at least 99% identity thereto. In some embodiments, the sequence encoding the first polypeptide comprises a sequence of SEQ ID NO: 18, or a sequence having at least 80%, at least 85%, at least 90%, at least 95% or at least 99% identity thereto. In some embodiments, the sequence encoding the second polypeptide comprises a sequence of SEQ ID NO: 19 or a sequence having at least 80%, at least 85%, at least 90%, at least 95% or at least 99% identity thereto.

The disclosure provides vectors comprising the polynucleotides of the disclosure.

In some embodiments of the vectors of the disclosure, the vector comprises a promoter operably linked to the sequence encoding the recombinant fusion protein or polynucleotide.

The disclosure provides pharmaceutical compositions comprising the recombinant fusion proteins of the disclosure and a pharmaceutically acceptable carrier, diluent or excipient.

In some embodiments of the pharmaceutical compositions of the disclosure, the pharmaceutical composition is suitable for parenteral administration. In some embodiments, the parenteral administration comprises intravenous infusion or injection, or subcutaneous injection.

The disclosure provides methods of treating a subject with a disease or disorder, comprising administering a therapeutically effective amount of the recombinant fusion proteins or pharmaceutical compositions of the disclosure.

In some embodiments of the methods of the disclosure, the disease or disorder is cancer. In some embodiments, the cancer comprises a solid tumor or a liquid tumor. In some embodiments, the liquid tumor comprises leukemia, acute myeloid leukemia, myeloma, acute myelogenous leukemia (AML), acute lymphoblastic leukemia (ALL), lymphoma, Hodgkin's lymphoma, non-Hodgkin's lymphoma, beta-cell lymphoma, chronic lymphocytic leukemia, chronic myelogenous leukemia, mantle cell lymphoma, follicular lymphoma, T-cell lymphoma, NK-cell lymphoma, B-cell lymphoma or NKT-cell lymphoma. In some embodiments, the cancer is selected from the group consisting of melanoma, renal cell carcinoma, mesothelioma, small cell lung cancer, uveal melanoma, bladder cancer, gastric cancer, squamous cell carcinoma of the head and neck, cutaneous carcinoma, non-small cell lung cancer, colorectal cancer, prostate cancer, ovarian cancer, cervical cancer, endometrial carcinoma, breast cancer, pancreatic cancer, urothelial cancer, hepatocellular carcinoma, esophageal cancer, glioblastoma, glioma, or sarcoma.

In some embodiments, the cancer is selected from the group consisting of melanoma, and renal cell carcinoma.

In some embodiments of the methods of the disclosure, the recombinant fusion protein or pharmaceutical composition inhibits the activity of CTLA-4 on an immune cell. In some embodiments, the recombinant fusion protein or pharmaceutical composition increases the activity of an Interleukin 2/Interleukin 15 receptor beta (IL-2Rb)/common gamma chain (IL-2RG) receptor complex on an immune cell. In some embodiments, the recombinant fusion protein or pharmaceutical composition promotes activity in an immune cell. In some embodiments, the activity comprises activation, proliferation, or a combination thereof. In some embodiments, the immune cell is a T cell, B cell or an NK cell. In some embodiments, the T cell is a CD8+ T cell. In some embodiments, the recombinant fusion protein or pharmaceutical composition increases proliferation of NK cells.

In some embodiments of the methods of the disclosure, the recombinant fusion protein or pharmaceutical composition is administered parenterally. In some embodiments, the parenteral administration comprises intravenous infusion or injection, or subcutaneous injection.

In some embodiments of the methods of the disclosure, administration of the recombinant fusion protein or pharmaceutical composition alleviates a sign or a symptom of the cancer. In some embodiments, administration of the recombinant fusion protein or pharmaceutical composition inhibits the progression of the cancer. In some embodiments, administration of the recombinant fusion protein or pharmaceutical composition prevents or delays recurrence of the cancer. In some embodiments, administration of the recombinant fusion protein or pharmaceutical composition induces partial or complete remission of the cancer.

In some embodiments of the methods of the disclosure, the methods comprise one or more additional cancer therapies. In some embodiments, the one or more additional cancer therapies comprises a chemotherapy, a small molecule inhibitor, a protein-based or biologic therapy, radiation, surgery, immunotherapy or adoptive cell therapy. In some embodiments, the adoptive cell therapy comprises a chimeric antigen receptor (CAR) T cell therapy, a T Cell Receptor (TCR) T cell therapy or a CAR NK cell therapy.

In some embodiments of the methods of the disclosure, administration of the recombinant fusion protein or pharmaceutical composition does not substantially increase a level of interferon gamma (IFNγ) in a peripheral blood sample from the subject. In some embodiments, administration of the recombinant fusion protein or pharmaceutical composition increases a level of interferon gamma (IFNγ) in a peripheral blood sample from the subject less than administration of an equimolar amount of IL-15 or IL-15 in a complex with the IL-15Ra sushi domain. In some embodiments, administration of the recombinant fusion protein or pharmaceutical composition increases proliferation of immune cells, but does not substantially increase a level of IFNγ in the subject. In some embodiments, the immune cells comprise NK cells, CD8+ T cells, or a combination thereof.

In some embodiments of the methods of the disclosure, administration of the recombinant fusion protein or pharmaceutical composition results in a ratio of IL-6 to IFNγ that is greater than or equal to 2:1, 3:1, 4:1, 5:1, 6:1, 7:1, 8:1, 9:1 or 10:1.

In some embodiments of the methods of the disclosure, administration of the recombinant fusion protein or pharmaceutical composition results in less toxicity than administration of an equimolar amount of IL-15 or IL-15 in a complex with the IL-15Ra sushi domain.

In some embodiments of the methods of the disclosure, the recombinant fusion protein is administered at a dose of 0.1 μg/kg to 1 mg/kg. In some embodiments, the recombinant fusion protein is administered at a dose of 10 μg/kg to 0.30 mg/kg.

In some embodiments of the methods of the disclosure, the recombinant fusion protein or pharmaceutical composition is administered intravenously, intratumorally or subcutaneously.

In some embodiments of the methods of the disclosure, the recombinant fusion protein or pharmaceutical composition is administered daily, every 2 days, every 3 days, every 4 days, every 5 days, every 6 days, every 7 days, every 8 days, every 9 days, every 10 days, every two weeks, every three weeks or monthly.

In some embodiments of the methods of the disclosure, the recombinant fusion protein or pharmaceutical composition is administered for at least one week, at least two weeks, at least three weeks, at least 4 weeks, at least 5 weeks, at least 6 weeks, at least 2 months, at least 3 months, at least 4 months, at least 5 months or at least 6 months, at least 8 months, at least 10 months, at least 12 months, at least 14 months, at least 16 months, at least 18 months, at least 20 months, at least 22 months or at least 2 years.

The disclosure provides recombinants fusion proteins or the pharmaceutical composition of the disclosure, for use in a method of treating of a disease or disorder in a subject.

The disclosure provides recombinants fusion proteins of the disclosure, for use the manufacture of a medicament for treating a disease or disorder in a subject.

The disclosure provides methods of making the recombinant fusion protein of the disclosure, comprising: (a) contacting a plurality of cells with the polynucleotides or vectors of the disclosure; (b) expressing the recombinant fusion protein by the plurality of cells; and (c) purifying the recombinant fusion protein.

The disclosure provides kits, comprising a therapeutically effective amount of the recombinant fusion proteins, the polynucleotides, the vectors, or the pharmaceutical compositions of the disclosure.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings wherein:

FIGS. 1A-1E are each a series of diagrams which show a CTLA-4 antibody, and exemplary CTLA-4 antibody fusion proteins of the disclosure.

FIG. 2 is a table comparing the thermal stability of two Fc variants of the anti-CTLA-4, IL-15Ra sushi domain, IL-15 fusion protein and a HER3 antibody-Neuregulin 1 fusion protein. WT: the knob heavy chain of the fusion protein has a S366W substitution, and the hole heavy chain has a Y407T substitution. WSAV: the knob heavy chain has a S366W substitution, and the hole heavy chain has T366S, L368A and Y407V substitutions.

FIG. 3 is a diagram showing the construction of expression vectors for the CTLA-4 antibody heavy chain with a “hole” modification in the constant region fused to the IL-15Ra sushi domain and IL-15, a CTLA-4 antibody heavy chain with a “knob” modification in the constant region, and a CTLA-4 antibody light chain.

FIG. 4 is a series of plots showing binding cross-reactivity of an anti CTLA-4 antibody (Ipilimumab) and the anti-CTLA-4-IL-15Ra-sushi-IL-15 fusion protein of the design shown in FIG. 1A to CTLA-4 derived from different species. αCTLA-4-IL-15Ra-IL-15: the anti-CTLA-4, IL-15Ra sushi domain, IL-15 fusion protein.

FIG. 5 is a plot showing the induction of interleukin 2 (IL-2) secretion by blockage of CTLA-4 receptor function in a cell co-culture assay system. αCTLA-4-IL15Ra-IL-15: the anti-CTLA-4, IL-15Ra sushi domain, IL-15 fusion protein.

FIG. 6 is a plot showing the antibody-dependent cellular cytotoxicity of the anti-CTLA-4-IL-15Ra-sushi-IL-15 fusion protein (SEQ ID NOS: 9, 10 and 16) compared to a CTLA-4 antibody.

FIG. 7 is a plot showing binding activity of the anti-CTLA-4-IL-15Ra-sushi-IL-15 fusion protein to the 1 subunit of interleukin 2 receptor (IL2Rβ).

FIGS. 8A-B are a pair of plots showing proliferation of wild type (FIG. 8A) and IL15Rα-deficient (FIG. 8B) T cells in response to stimulation with the anti-CTLA-4-IL-15Ra-sushi-IL-15 fusion protein. αCTLA-4-IL-15Ra-IL-15: the anti-CTLA-4, IL-15Ra sushi domain, IL-15 fusion protein; αCTLA-4-IL-15: CTLA-4 antibody fused to IL-15, no IL-15Ra sushi domain, as shown in FIG. 1A.

FIGS. 9A-9D are each a plot showing proliferation of wild type (CTLL2-WT, FIGS. 9A and 9C) and IL15Rα-deficient (CTLL2-IL15RAKO, FIGS. 9B and 9D) T cells in response to stimulation with various anti-CTLA-4-IL-15Ra-sushi-IL-15 fusion constructs of the disclosure. Construct schematics are as shown in FIGS. 1B-1E. BS3hole in FIG. 9D refers to a construct with the BS3 architecture shown in FIG. 1E, but with two hole heavy chains fused to IL-15Ra-sushi_IL-15 instead of the knob and hole heavy chains.

FIGS. 10A-1OC are each a pair of plots showing NK cell and CD8⁺ T cell proliferation in response to administration of the anti-CTLA-4-IL-15Ra-sushi-IL-15 fusion protein in C57BL6 mice. αCTLA-4-IL-15Ra-IL-15: CTLA-4 antibody fused to IL-15Ra sushi domain and IL-15; αCTLA-4-1L-15: CTLA-4 antibody fused to IL-15, no sushi domain.

FIG. 11 is a series of plots showing expansion of NK cells, CD8⁺ T cells, and CD4⁺ T cells in response to treatment with the anti-CTLA-4-IL-15Ra-sushi-IL-15 fusion protein (of SEQ ID NOS: 9, 10 and 16) in cynomolgus macaques.

FIG. 12 is a plot showing cytokine induction in response to administration of the anti-CTLA-4-IL-15Ra-sushi-IL-15 fusion protein in cynomolgus macaques. Units on the y axis are in picograms (pg) per milliliter (mL). Cytokine levels were assayed 3 days prior to administration (D-3), and then 2 hours, 24 hours and 48 hours after the first administration (D1(2h), D1(24h) and D1(48h) respectively) in a once-weekly four week repeat-dose toxicology study, and again at 2 hours, 24 hours and 48 hours ((D22(2h), D22(24h) and D22(48h) respectively) after the fourth dose in a once-weekly four week repeat-dose toxicology study.

FIG. 13A is a pair of plots showing the anti-tumor activity following treatment with anti-CTLA-4-IL-15Ra-sushi-IL-15 fusion protein (of SEQ ID NOS: 9, 10 and 16) in mice expressing human CTLA-4 and bearing MC38 xenograft tumors.

FIG. 13B is a pair of plots showing the anti-tumor activity following treatment with anti-CTLA-4-IL-15Ra-sushi-IL-15 fusion protein (of SEQ ID NOS: 9, 10 and 16) in mice expressing human CTLA-4 and bearing B16F10 xenograft tumors.

### DETAILED DESCRIPTION

Interleukin-15 (IL-15) is a common gamma chain cytokine that plays a role in the development, survival, proliferation and activation of lymphocytes, including natural killer (NK) cells, T cells such as CD8+αβ T cells, γδ T cells and NKT cells, and intraepithelial T lymphocytes. IL-15 shares a common gamma chain with receptor with IL-2, and has similar biological effects. IL-15 is co-produced in cells with a second polypeptide, IL-15 Receptor alpha (IL-15Ra), and the two proteins form stable heterodimers which are transported to the plasma membrane where the IL-15Ra to release a soluble heterodimer into the extracellular space and plasma circulation.

Unlike IL-2, IL-15 has been shown to promote the cytotoxic immune response without also promoting activation-induced cell death, a mechanism by which the risk of autoimmunity through the elimination of self-reactive T cells is reduced. In mouse cancer models, administration of IL-15 has been shown to enhance the in vivo anti-tumor activity of CD8+ T cells, and prolong survival. Without wishing to be bound by theory, it is thought that IL-15 induces lymphocyte entry into tumors, and increases their cytotoxicity, as well as affecting proliferation and homeostasis. Thus, recombinant IL-15, either as a monomer or as the soluble heterodimer, is an attractive target as a cancer therapeutic.

However, administration of recombinant IL-15, either as a monomer or as a heterodimer, is associated with significant toxicity. Administration of recombinant human IL-15 to human cancer patients by daily intravenous bolus resulted in marked increases in the levels of IL-6, IL-8, and IFNγ, as well as IL-10, tumor necrosis factor α, and IL-1p. These increases coincided with clinical toxicities such as fever, chills, rigors and blood pressure changes, leading the study authors to conclude that recombinant human IL-15 was too difficult to administer as an intravenous bolus dose (Conlon et al. (2015) Journal of Clinical Oncology 33: 74-82). Similarly, when IL-15 complexed with IL-15Ra was administered to mice, significant toxicity was observed, including hypothermia, weight loss, acute liver injury and mortality (Guo et al. (2015) J Immunol 1953:2353-2364). The toxic effects of IL-15/IL-15Ra heterodimer appeared to be mediated primarily by the expansion and activation of NK cells, which in turn resulted from the increased expression of interferon gamma (IFNγ) that occurred following administration of the IL-154IL-15Ra heterodimer. Similarly, when 14 patients with metastatic or unresectable solid tumors were treated with IL-15-IL-15Ra heterodimer in an escalating dose study, serious adverse events were observed in three patients, and included dermatitis bullous, purpura and acute kidney injury (Conlon et al. J. Immunother Cancer 2021, 9:e003388). Induction of several cytokines, including IFNγ, was also observed, and the fold-increase of IFNγ exceeded the fold-increase of all other cytokines measured.

There thus exists a need for improved IL-15 based therapeutics with reduced toxicity compared to the recombinant IL-15 or IL-15 complexed with IL-15Ra described supra. The inventors have unexpectedly found that IL-15 fused to the IL-15Ra sushi domain, when also fused to a CTLA-4 antigen binding domain, does not lead to increased levels of IFNγ. Thus, the disclosure provides fusion proteins comprising an CTLA-4 antigen binding domain, an IL-15Ra sushi domain and IL-15 that have superior safety with retained pharmacodynamic activity compared to the recombinant human IL-15 or L-15/IL-15Ra heterodimer known in the art.

Cytotoxic T-lymphocyte-associated protein 4 (CTLA-4) is a transmembrane receptor that functions as an immune checkpoint and downregulates immune responses. CTLA-4 is homologous to CD28, a critical T cell co-stimulatory receptor for T cell activation. Both CTLA-4 and CD28 molecules can bind to CD80 and CD86 on antigen-presenting cells in a competitive manner, thereby modulating immune responses in which CTLA-4 transmits an inhibitory signal to T cells, whereas CD28 transmits a stimulatory signal. CTLA-4 binds CD80 and CD86 with greater affinity than CD28 thus enabling it to outcompete CD28 for its ligands. CTLA-4 is constitutively expressed in regulatory T cells but only upregulated in effector T cells after activation. The anti-CTLA-4 antibody ipilimumab is the first immune checkpoint inhibitor therapy for cancer approved by the FDA. Despite intensive investigation, the molecular mechanism by which ipilimumab exerts its immunotherapeutic effect remains a subject of debate. Although the initial premise was that anti-CTLA-4 antibodies function by blocking inhibitory signals into effector T cells, recent studies suggested that the in vivo anti-tumor activity of ipilimumab may be attributed to the depletion of regulatory T cells through the ADCC mechanism (Simpson T. R., et al., 2013, J. Exp. Med. 210(9):1695-1710; Du X., et al., 2018, Cell Research 0:1-15). Without wishing to be bound by theory, it is thought that IL-15 can enhance the ability of a CTLA-4 antibody to deplete regulatory T cells through its ability to expand CD8 T cells and NK cells.

Accordingly, provided herein is a recombinant fusion protein comprising an antibody that binds to cytotoxic T-lymphocyte associated protein 4 (CTLA-4, also known as CD152), a sushi domain of the interleukin 15 receptor alpha chain (IL-15Ra, or IL-15Ra) and interleukin 15 (IL-15). Provided herein are polynucleotides and vectors encoding these recombinant fusion proteins, as well as pharmaceutical compositions comprising the recombinant fusion proteins, and methods of making and using same. The recombinant fusion protein can be used to treat a variety of diseases and disorders, including cancers.

### Definitions

Unless defined otherwise, technical and scientific terms used herein have the same meaning as commonly understood by one of ordinary skill in the art to which this invention belongs.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The term “active,” as used herein, refers to a fragment having a biological activity or biological function. In some embodiments, the activity is equal to or approximates the activity of the wild-type protein.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, a non-mammal, including, e.g., a non-mammalian vertebrate, such as a bird (e.g., a chicken or duck) or a fish; and a non-mammalian invertebrate. In some embodiments, the methods and compositions of the invention are used to treat (both prophylactically and/or therapeutically) non-human animals. The term “subject” can also refer to patients, i.e. individuals awaiting or receiving medical care.

The term “pharmaceutical composition” herein means a composition suitable for pharmaceutical use in a subject, including an animal or human. A pharmaceutical composition generally comprises an effective amount of an active agent (e.g., the recombinant fusion proteins of the invention) and a pharmaceutically acceptable carrier, diluent or excipient (e.g., a buffer, adjuvant, or the like).

The term “effective amount” means a dosage or amount sufficient to produce a desired result. The desired result may comprise an objective or subjective improvement in the recipient of the dosage or amount (e.g., long-term survival, decrease in number and/or size of tumors, effective prevention of a disease state, etc.).

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as the recombinant fusion protein of the invention, or composition thereof, that, when administered to a subject who does not display signs or symptoms of a pathology, disease or disorder (or who displays only early signs or symptoms of a pathology, disease, or disorder) diminishes, prevents, or decreases the risk of the subject developing the pathology, disease, or disorder. A “prophylactically useful” agent or compound (e.g., a recombinant fusion protein of the invention) refers to an agent or compound that is useful in diminishing, preventing, treating, or decreasing development of a pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such a recombinant fusion protein of the invention, or a composition thereof, that eliminates or diminishes signs or symptoms of a pathology, disease or disorder, when administered to a subject suffering from such signs or symptoms. A “therapeutically useful” agent or compound (e.g., a recombinant fusion protein of the invention) indicates that an agent or compound is useful in diminishing, treating, or eliminating such signs or symptoms of the pathology, disease or disorder.

The term “treating cancer” as used herein, unless otherwise indicated, means reversing, alleviating, inhibiting the progress of, or preventing, either partially or completely, the growth of tumors, tumor metastases, or other cancer-causing or neoplastic cells in a subject. The term “treatment” as used herein, unless otherwise indicated, refers to the act of treating.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity equals the number of identical positions/total number of positions (e.g., overlapping positions)×100). In some embodiments, the two sequences are the same length.

The term “substantially identical,” in the context of two nucleic acids or polypeptides, refers to two or more sequences or subsequences that have at least 80%, at least 81%, at least 82%, at least 83%, at least 84%, at least 85%, at least 86%, at least 87%/o, at least 88%, at least 890%, 90%, at least 91%, at least 92%, at least 93%, at least 94%, at least 95%, at least 96%, at least 97%, at least 98% identity, or at least 99% identity (e.g., as determined using one of the methods set forth infra).

The determination of percent identity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search, which detects distant relationships between molecules (id.). When utilizing BLAST, Gapped BLAST, and PSI-BLAST programs, the default parameters of the respective programs (e.g., XBLAST and NBLAST) can be used. Another non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Alternatively, protein sequence alignment may be carried out using the CLUSTAL W algorithm, as described by Higgins et al., 1996, Methods Enzymol. 266:383-402.

As used herein, the term binds,” “specifically binds to,” or is “specific to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (Kd) of <1 μM, <100 nM, <10 nM, <1 nM, or <0.1 nM.

In certain embodiments, an antibody specifically binds to an epitope on a protein that is conserved among the protein from different species. In another embodiment, specific binding can include, but does not require exclusive binding.

As used in this specification, the singular forms “a”, “an”, and “the” include plural references unless the context clearly dictates otherwise. Reference to “the formulation” or “the method” includes one or more formulations, methods, and/or steps of the type described herein and/or which will become apparent to those persons skilled in the art upon reading this disclosure.

The term “polypeptide” refers to a polymer of amino acids and its equivalent and does not refer to a specific length of a product; thus, “peptides” and “proteins” are included within the definition of a polypeptide. A protein can have one or more polypeptides. Also included within the definition of polypeptides are “antibodies” as defined herein. A “polypeptide region” refers to a segment of a polypeptide, which segment may contain, for example, one or more domains or motifs (e.g., a polypeptide region of an antibody can contain, for example, one or more complementarity determining regions (CDRs)). The term “fragment” refers to a portion of a polypeptide that is less than the entire polypeptide, as it occurs naturally.

Unless otherwise indicated by context, a “derivative” is a polypeptide or fragment thereof having one or more non-conservative or conservative amino acid substitutions relative to a second polypeptide (also referred to as a “variant”); or a polypeptide or fragment thereof that is modified by covalent attachment of a second molecule such as, e.g., by attachment of a heterologous polypeptide, or by glycosylation, acetylation, phosphorylation, and the like. Further included within the definition of “derivative” are, for example, polypeptides containing one or more analogs of an amino acid (e.g., unnatural amino acids and the like), polypeptides with unsubstituted linkages, as well as other modifications known in the art, both naturally and non-naturally occurring.

An “isolated” polypeptide is one which has been identified and separated and/or recovered from a component of its natural environment. Contaminant components of its natural environment are materials which would interfere with diagnostic or therapeutic uses for the polypeptide, and may include enzymes, hormones, and other proteinaceous or nonproteinaceous solutes. An isolated polypeptide includes an isolated antibody, or a fragment or derivative thereof.

T cells are a type of lymphocyte that express a T Cell Receptor (TCR) on their cell surface, and play a central role in the adaptive immune response. T cells are produced by hematopoietic stem cells in in the bone marrow, and migrate to the thymus gland to mature. Types of T cells include CD4+ helper T cells, cytotoxic T cells, memory T cells, and NKT cells. Types of T cells will be readily apparent to the person of ordinary skill in the art by their expression of combinations of markers, such as CD4, CD8 and CD45RO.

Natural Killer (NK) cells are a type of cytotoxic lymphocyte that play a role in the innate immune response. NK cells can recognize and kill stressed cells in the absence of antibodies and or major histocompatibility complex (MHC) expression, producing a fast immune response. In addition, antibodies that bind to antigens can be recognized by FcγRIII (CD16) receptors expressed on NK cells, resulting in NK activation, release of cytolytic granules and cell apoptosis. Like T cells, NK cells differentiate from hematopoietic stem cells. NK cells will be apparent to the person of ordinary skill in the art through their expression of markers or combinations of markers, for example CD56+ and CD3−.

To activate T cells and NK cells means to induce a change in their biologic state by which the cells express activation markers, produce cytokines, proliferate and/or become cytotoxic to target cells.

For T-cells, engagement of the T-cell receptor (TCR) alone is usually not sufficient to induce persistent activation of resting naive or memory T cells. Full T cell activation requires a second co-stimulatory signal from a competent antigen-presenting cell (APC). Co-stimulation is achieved naturally by the interaction of the co-stimulatory cell surface receptor on the T cell with the appropriate counter-receptor on the surface of the APC. An APC is normally a cell of host origin which displays a moiety which will cause the stimulation of an immune response. APCs include monocyte/macrophages, dendritic cells, B cells, and any number of virally-infected or tumor cells which express a protein on their surface recognized by T cells. To be immunogenic APCs must also express on their surface a co-stimulatory molecule. Such APCs are capable of stimulating T cell proliferation, inducing cytokine production, and acting as targets for cytolytic T cells upon direct interaction with the T cell.

For NK cells, activation is determined at least in part by the balance of inhibitory and activating receptor stimulation. Exemplary activating receptors include Ly49, NCR receptors and CD16, while exemplary inhibitory receptors include the Killer-cell immunoglobulin-like receptors (KIRs), CD94/NKG2, and LIR. Cytokines play a role in NK cell activation. Cytokines, which are released by cells under stress, for example the stress of an infection, NK cell the presence of pathogens in the affected area. Cytokines involved in NK activation include IL-12, IL-15, IL-18, IL-2, and CCL5. NK cells are also activated in response to interferons or macrophage-derived cytokines.

B cells, also known as B lymphocytes, are a type of white blood cell of the lymphocyte subtype. They function in the humoral immunity component of the adaptive immune system by secreting antibodies.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “about” as used herein means in quantitative terms plus or minus 5%, or in another embodiment plus or minus 10%, or in another embodiment plus or minus 15%, or in another embodiment plus or minus 20%.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications, patents, and patent applications mentioned in this specification are herein incorporated by reference to the same extent as if each individual publication, patent, or patent application was specifically and individually indicated to be incorporated by reference.

### Recombinant Fusion Proteins

The disclosure provides a recombinant fusion protein comprising an interleukin 15 (IL-15) domain; an Interleukin 15 receptor subunit alpha (IL-15Ra) sushi domain, and an antigen binding domain specific to cytotoxic T-lymphocyte associated protein 4 (CTLA-4).

### Interleukin 15 (IL-15)

The disclosure provides a recombinant fusion protein comprising Interleukin 15 (IL-15), or an active fragment or derivative thereof.

IL-15 is an immunoregulatory cytokine that belongs to the family of cytokines that includes Interleukin-2 (IL-2), Interleukin-4 (IL-4), Interleukin-7 (IL-7), Interleukin-9 (IL-9), and Interleukin-21 (IL-21). Like IL-2, IL-15 binds to and signals through a receptor complex comprising the IL-2/1L-15 receptor p (IL-2RO, or LL-2Rb, also called CD122) subunit, and the common gamma chain (yC) (IL-2RG, or CD132) receptor subunit. IL-15 has multiple functions, including, but not limited to, regulating T cell response, regulating tissue repair and B cell homing, modulating inflammation, and activating NK cells. IL-15 signaling can stimulate an array of downstream pathways leading to increased cellular growth, decreased apoptosis, and enhanced immune cell activation and migration. IL-15 is also thought to play a role in NKT cell development and survival. IL-15 can stimulate the proliferation, survival and cytotoxic functions of T cells and NK cells, and induce the generation of cytotoxic lymphocytes, leading to enhanced anti-tumor responses.

IL-15 is 14-15 kDa glycoprotein. The human IL-15 gene comprises nine exons (1-8 and 4A) and eight introns, four of which (exons 5 through 8) encode the mature protein. Two alternatively spliced transcript variants of IL-15, which differ in their cellular trafficking but encode the same mature protein, have been reported. The IL-15 gene is described, for example, in NCBI record NG_029605.2, the contents of which are incorporated by reference in their entirety herein.

In some embodiments, the IL-15 domain of the recombinant fusion protein described herein is active. IL-15 activities include, but are not limited to, promoting immune cell activation, promoting immune cell proliferation, decreasing immune cell apoptosis, regulating immune cell response, regulating immune cell release of cytokines, and regulating immune cell differentiation. In some embodiments, IL-15 activities comprise promoting immune cell activation, promoting immune cell proliferation, or a combination thereof. In some embodiments, the immune cells comprise T cells, B cells, NK cell or a combination thereof.

In some embodiments, the IL-15 domain comprises a sequence of NWVNVISDLKKIEDLIQSMHIDATLYTESDVHPSCKVTAMKCFLLELQVISLESG DASIHDTVENLIULANNSLSSNGNVTESGCKECEELEEKNIKEFLQSFVHIVQMFIN TS (SEQ ID NO: 1), or a sequence having at least 80%, at least 85%, at least 90%, at least 95%, at least 97%, or at least 99% identity thereto. In some embodiments, the IL-15 domain comprises a sequence of SEQ ID NO: 1, or a sequence having at least 90%,o, at least 95%, at least 97%, or at least 99% identity thereto. In some embodiments, the IL-15 domain comprises, or consists essentially of, SEQ ID NO: 1.

In some embodiments, the IL-15 domain is encoded by a sequence comprising

(SEQ ID NO: 21)
AATTGGGTCAACGTGATCTCCGACCTGAAGAAGATCGAGGACCTGATCCA
GTCCATGCACATCGACGCTACCCTGTACACCGAGTCCGACGTGCACCCTT
CCTGTAAAGTGACCGCCATGAAGTGCTTTCTGCTGGAACTGCAAGTGATC
TCCCTGGAATCCGGCGACGCCTCTATCCACGACACCGTGGAAAACCTGAT
CATCCTGGCCAACAACTCCCTGTCCTCCAACGGCAACGTGACCGAGTCTG
GCTGCAAAGAGTGCGAGGAACTGGAAGAGAAGAACATCAAAGAGTTCCTC
CAGTCCTTCGTGCACATCGTGCAGATGTTCATCAACACCAGC,

or a sequence having at least 80%, at least 85%, at least 90%, at least 95%, at least 97%, at least 99% or is identical thereto. In some embodiments, the IL-15 domain is encoded by a sequence comprising SEQ ID NO: 21, or a sequence having at least 90%, at least 95%, at least 97%, at least 99% or is identical thereto. In some embodiments, the IL-15 domain is encoded by a sequence comprising SEQ ID NO: 21.

### IL-15Ra Sushi Domain

The disclosure provides a recombinant fusion protein comprising IL-15, an IL-15Ra sushi domain, and an anti-CTLA-4 antibody.

Interleukin 15 receptor subunit alpha (IL-15Ra or LL-15Ra) is a critical component of the IL-15 cytokine-receptor complex. IL-15Ra is a transmembrane protein with very high affinity for IL-15 that facilitates IL-15 trafficking from the endoplasmic reticulum (ER) through the cytoplasm and presentation of IL-15/IL-15Ra complexes on the cell surface. In addition to remaining associated throughout cytoplasmic and cell surface expression, IL-15/IL-15Ra can also be cleaved as a complex into the extracellular space. These peculiarities of IL-15 and IL-15R subunits lend itself to unique mechanisms of cytokine delivery. In contrast to the selective expression of the signaling subunits for IL-15, the cytokine itself and IL-15Ra are widely expressed by most cell types, including both hematopoietic and non-hematopoietic cells but are highest among myeloid cells.

The IL-15Ra sushi domain is an extracellular protein-protein interacting domain that contains four cysteines forming two disulfide bonds in a 1-3 and 2-4 pattern. Without wishing to be bound by theory, it is thought that the IL-15Ra sushi domain acts as an IL-15 agonist by enhancing IL-15 binding to, and effects on, immune cells through the 1L-2R beta/AL-2R gamma heterodimer receptor complex.

In some embodiments, the IL-15Ra sushi domain increases the activity of the IL-15 domain compared to the activity of an IL-15 domain in an otherwise equivalent recombinant fusion protein lacking the IL-15Ra sushi domain. For example, the presence of the IL-15Ra sushi domain as part of the recombinant fusion protein described herein can increase the effect of recombinant fusion protein, of which the IL-15 domain is a part, on immune cell proliferation, activation, or a combination thereof.

Human IL-15Ra is described, for example, at UniProtKB record Q13261.1, the contents of which are incorporated by reference in their entirety. In some embodiments, IL-15Ra comprises a sequence of:

(SEQ ID NO: 20)

| | |
|---|---|
| 1 | MAPRRARGCR TLGLPALLLL LLLRPPATRG ITCPPPMSVE |
| | HADIWVKSYS LYSRERYICN |
| 61 | SGFKRKAGTS SLTECVLNKA TNVAHWTTPS LKCIRDPALV |
| | HQRPAPPSTV TTAGVTPQPE |
| 121 | SLSPSGKEPA ASSPSSNNTA ATTAAIVPGS QLMPSKSPST |
| | GTTEISSHES SHGTPSQTTA |
| 181 | KNWELTASAS HQPPGVYPQG HSDTTVAIST STVLLCGLSA |
| | VSLLACYLKS RQTPPLASVE |
| 241 | MEAMEALPVT WGTSSRDEDL ENCSHHL . |

In SEQ ID NO: 20, supra, the sushi domain is underlined. The person of ordinary skill in the art will understand that fragments of IL-15Ra encompassing all or part of the sushi domain that differ at the N and C termini by 1, 2, 3, 4, 5 or more amino acids may have sushi domain activity, and are envisaged as within the scope of the instant invention.

In some embodiments, the IL-15Ra sushi domain comprises a sequence of comprises a sequence of ITCPPPMSVEHADIWVKSYSLYSRERYICNSGFKRKAGTSSLTECVLNKATNVAH WTTPSLKCIRDPALVHQRPAPPSTV (SEQ ID NO: 2), or a sequence having at least 80%, at least 85%, at least 90%, at least 95%, at least 97%, or at least 99% identity thereto. In some embodiments, the IL-15Ra sushi domain comprises a sequence of comprises a sequence of SEQ ID NO. 2, or a sequence having at least 90%, at least 95%, at least 97%, or at least 99% identity thereto. In some embodiments, the IL-15Ra sushi domain comprises, or consists essentially of, a sequence of SEQ ID NO: 2.

In some embodiments, the IL-15Ra sushi domain is encoded by a sequence comprising ATTACATGCCCTCCTCCAATGTCCGTGGAACACGCCGACATCTGGGTCAAGTCCTACAG CCTGTACTCCAGAGAGCGGTACATCTGCAACTCCGGCTTCAAGAGAAAGGCCGGCACCT CTAGCCTGACCGAGTGCGTGCTGAACAAGGCCACCAATGTGGCCCACTGGACCACACCT AGCCTGAAGTGCATCAGGGACCCCGCTCTGGTTCATCAGAGGCCTGCTCCTCCATCTAC CGTT (SEQ ID NO: 22), or a sequence having at least 80%, at least 85%, at least 90%, at least 95%, at least 97%, at least 99% or is identical thereto. In some embodiments, the IL-15Ra domain is encoded by a sequence comprising SEQ ID NO: 22, or a sequence having at least 90%, at least 95%, at least 97%, at least 99% or is identical thereto. In some embodiments, the IL-15Ra domain is encoded by a sequence comprising SEQ ID NO: 22.

### CTLA-4 Antigen Binding Domains

The disclosure provides fusion proteins comprising an antigen binding domain specific to CTLA-4 (sometimes referred to herein as a CTLA-4 antigen binding domain). Any suitable CTLA-4 antigen binding domain is envisaged within the scope of the instant disclosure, including, but not limited to, single chain variable fragments (scFv), single domain antibodies (sdAb) such as VHH single domain antibodies, antibodies, or antibody fragments.

Human CTLA-4, also known as CD152, is a member of the membrane-bound single V domain subfamily within the immunoglobulin superfamily that is found primarily on activated T cells and regulatory T cells. T-lymphocytes (T cells) are central to the adaptive immune response to antigen. At least two signals are required for full activation of naive T-cells. A first, antigen-specific signal is provided by interaction of the T-cell receptor (TCR) with MHC/peptide complex on an antigen-presenting cell (APC). A second, co-stimulatory signal is provided by the interactions between receptors on the T-cell and their ligands on an antigen presenting cell (APC). Engagement of both TCR/MHC and co-stimulatory interactions leads to T-cell activation via a number of intracellular pathways, and subsequent activation of transcription factors for a number of effector compounds, including cytokines such as IL-2. These events lead to T-cell proliferation, generation of a CD4⁺ helper T-cell (T^{H}) pool, and expansion of activated CD8⁺ cytotoxic T-cells. Not only is co-stimulation critical for full T-cell activation, its absence during TCR/MHC engagement results in anergy and/or apoptosis. One critical interaction takes place between CD28 on T-cells and B7-1 (CD80) and B7-2 (CD86) on APCs. CD28 promotes T-cell differentiation into TH1 phenotype cells and enhances antibody production by B cells and activation of T-cells. After T-cell activation, CTLA-4, which functions as a negative regulatory receptor, is upregulated on T-cells. CTLA-4 inhibits the immune response in several ways: it competes with CD28 for the B7 ligands and thus blocks co-stimulation; it negatively signals to inhibit T-cell activation; and it can capture CD80 and CD86 from opposing cells by trans-endocytosis, resulting in impaired costimulation via CD28. CTLA-4 functions as an immune checkpoint, and activation of CTLA-4 leads to downregulation of the immune response. By antagonizing CTLA-4 activation, for example through use of an antigen binding domain specific to CTLA-4 that acts as a CTLA-4 antagonist, it is possible to prevent or reduce CTLA-4 mediated downregulation of the immune response.

In some embodiments, an antigen binding domain of the disclosure specific to CTLA-4 can act as a CTLA-4 antagonist. In some embodiments, the CTLA-4 antigen binding domain prevents or reduces CTLA-4 mediated downregulation of the immune response. CTLA-4 antagonists can reduce the development of immune system tolerance, for example to cancers and infections, and promote activities of immune cells. For example, CTLA-4 antagonists can promote immune cell activation and proliferation.

In some embodiments, the antigen binding domain specific to CTLA-4 is an antibody, for example a monoclonal antibody.

The term “antigen-binding region” as used herein refers to a domain of an antigen binding moiety that is responsible for the specific binding between an antigen binding moiety and an antigen. For example, the antigen-binding region of an antibody or a fragment thereof is formed by amino acid residues of the N-terminal variable regions of the heavy chain (abbreviated herein as VH) and the light chain (abbreviated herein as VL). The variable regions of the VH and the VL each comprise three hypervariable regions, termed complementary determining regions (CDR). The 3 CDRs of the VH and the 3 CDRs of the VL are three-dimensionally disposed relative to each other to form an antigen binding surface.

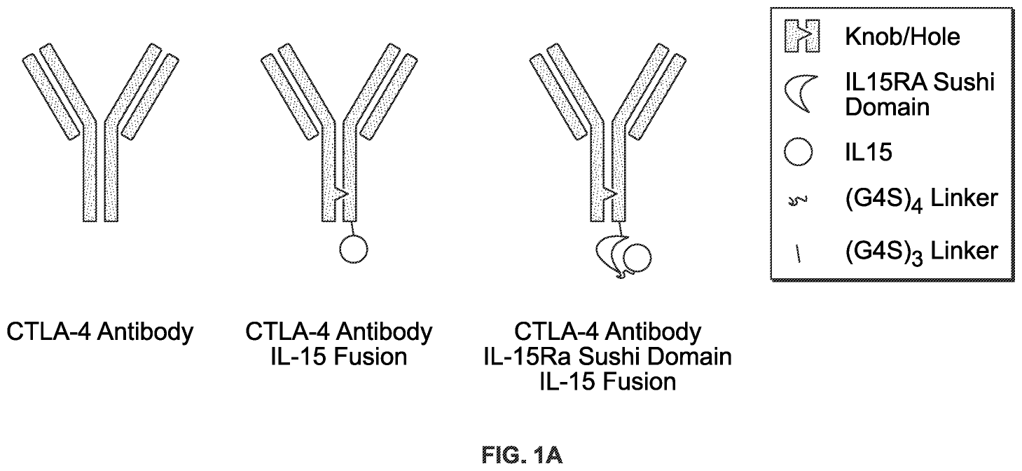

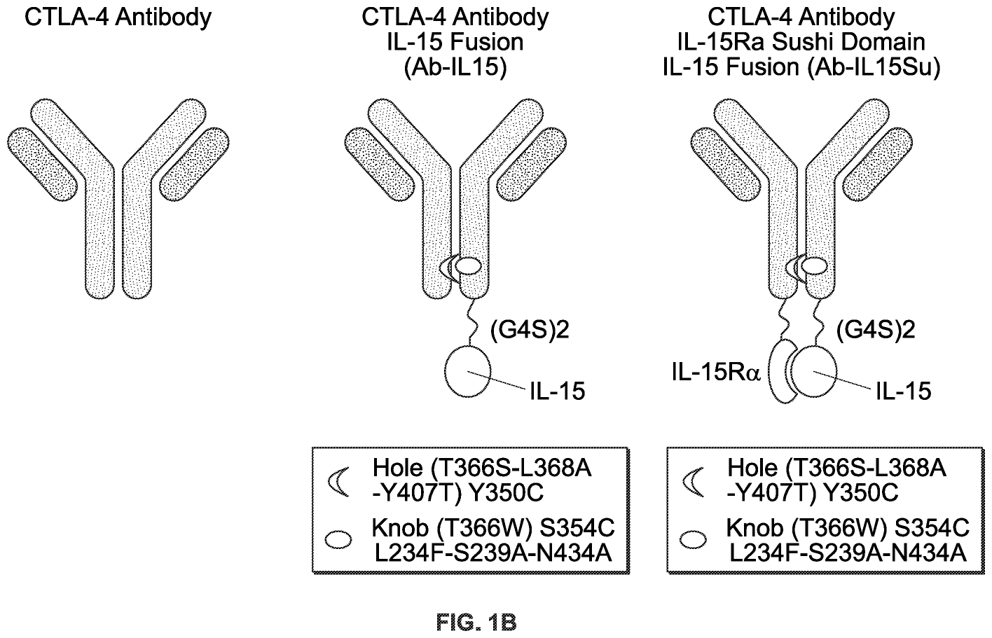

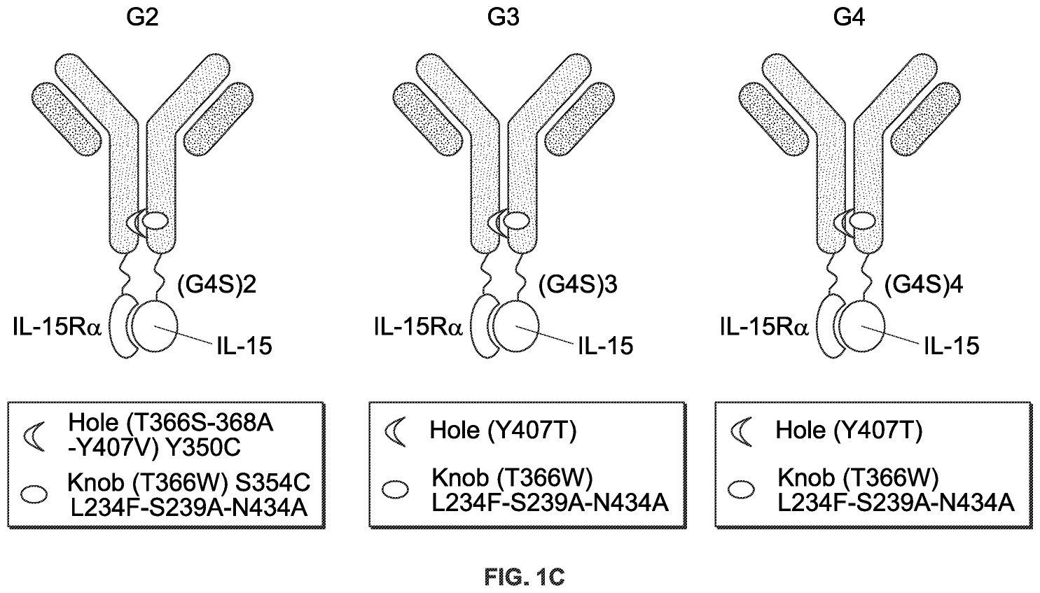

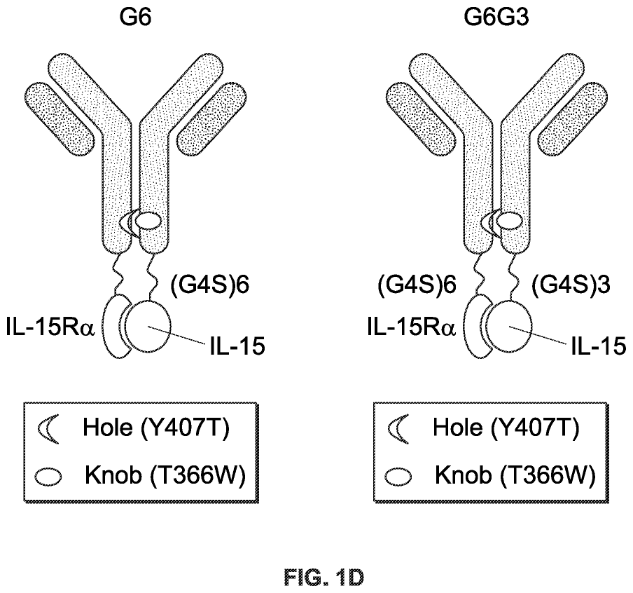

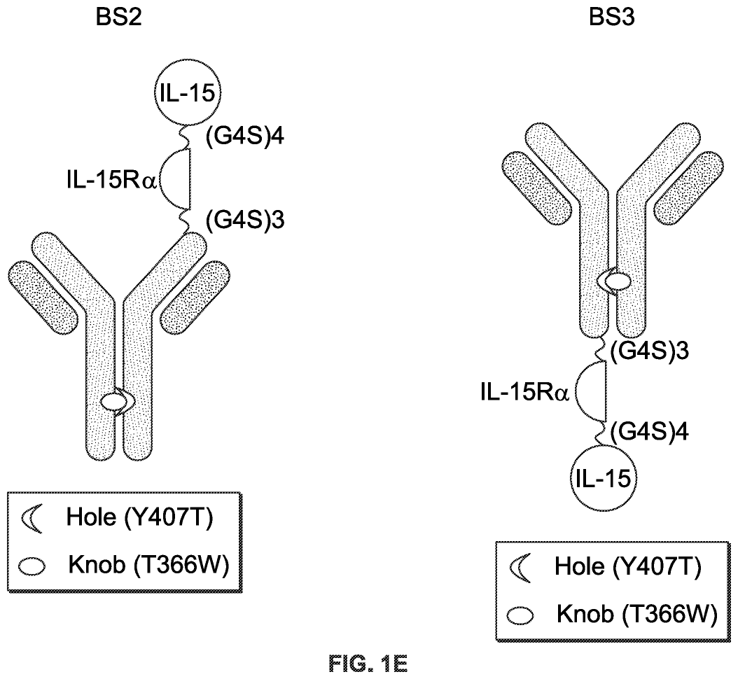

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD), as shown in FIG. 1A. The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the F(ab′)2dimer into an Fab′ monomer. The Fab′ monomer is essentially a Fab with part of the hinge region (see, Fundamental Immunology, W. E. Paul, ed., Raven Press, New York (1999), for a more detailed description of other antibody fragments). While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies.

A naturally occurring “antibody” is a protein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementary determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The constant regions of the antibodies may mediate the binding of the immunoglobulin to host tissues or factors, including various cells of the immune system (e.g., effector cells) and the first component (C1q) of the classical complement system.

Antibodies include single chain antibodies, including single chain Fv (sFv or scFv) antibodies in which a variable heavy and a variable light chain are joined together (directly or through a peptide linker) to form a continuous polypeptide.

Antibodies include single domain antibodies, which comprise an antibody fragment consisting of a single monomeric variable antibody domain that is able to bind selectively to an antigen domain. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies Single domain antibodies may be derived from any species including, but not limited to mouse, human, camel, llama, goat, rabbit. bovine According to one aspect of the invention, a single domain antibody as used herein is a naturally occurring single domain antibody known as heavy chain antibody devoid of light chains. Such single domain antibodies are disclosed in WO 9404678 for example. For clarity reasons, this variable domain derived from a heavy chain antibody naturally devoid of light chain is known herein as a VHH to distinguish it from the conventional VH of four chain immunoglobulins Such a VHH molecule can be derived from antibodies raised in Camelidae species. for example in camel, llama, dromedary, alpaca and guanaco.

The antibody domain of the fusion protein optionally comprises all or part of an immunoglobulin molecule and optionally contains all or part of an immunoglobulin variable region (i.e., the area of specificity for the disease related antigen) and optionally comprises region(s) encoded by a V gene, and/or a D gene and/or a J gene.

As explained above (see, Definitions, supra) the antibodies used herein optionally comprise F(ab)2, F(ab′)2, Fab, Fab′, scFv, single domain antibodies, etc. depending upon the specific requirements of the embodiment. Some embodiments utilize fusion proteins comprising IgG domains. However, other embodiments comprise alternate immunoglobulins such as IgM, IgA, IgD, and IgE. Furthermore, all possible isotypes of the various immunoglobulins are also encompassed within the current embodiments. Thus, IgG1, IgG2, IgG3, etc. are all possible molecules in the antibody domains of the antibody fusion proteins used in the invention. In addition to choice in selection of the type of immunoglobulin and isotype, different embodiments of the invention comprise various hinge regions (or functional equivalents thereof). Such hinge regions provide flexibility between the different domains of the antibody fusion proteins. See, e.g., Penichet, et al. 2001 “Antibody-cytokine fusion proteins for the therapy of cancer” J Immunol Methods 248:91-101.

In some embodiments, the CTLA-4 antigen binding domain comprises a light chain variable region and a heavy chain variable region. In some embodiments, the heavy chain variable region comprises complementarity determining region (CDR) sequences of GFTFSSYT (SEQ ID NO: 5), ISYDGNNK (SEQ ID NO: 6) and ARTGWLGPFDY (SEQ ID NO: 7). In some embodiments, the light chain variable region comprises CDR sequences of QSVGSSY (SEQ ID NO: 3), GAF and QQYGSSPWT (SEQ ID NO: 4). In some embodiments, the CTLA-4 antigen binding domain comprises a light chain and a heavy chain. In some the heavy chain comprises CDR sequences of SEQ ID NO: 5, SEQ ID NO: 6 and SEQ ID NO: 7. In some embodiments, the light chain comprises CDR sequences of SEQ ID NO: 3, GAF and SEQ ID NO: 4. In some embodiments, the CTLA-4 antigen binding domain comprises a heavy chain comprising CDR sequences of SEQ ID NOS: 5, 6, and 7, and a light chain comprising CDR sequence of SEQ ID NO: 3, GAF and SEQ ID NO: 4.

Exemplary, but non-limiting CTLA-4 CDR sequences are shown in Table 1. Below.

TABLE 1
CDR sequences of antibodies specific to CTLA-4

| CDR-L1 | CDR-L2 | CDR-L3 | CDR-H1 | CDR-H2 | CDR-H3 |
|---|---|---|---|---|---|
| QSVGSSY | GAF | QQYGSSP | GFTFSSY | ISYDGNNK | ARTGWLGP |
| (SEQ ID | | WT (SEQ | T (SEQ | (SEQ ID | FDY (SEQ |
| NO: 3) | | ID NO: | ID NO: | NO: 6) | ID NO: 7) |
| | | 4) | 5) | | |
| QSINSY | AAS | QQYYSTPF | GFTFSSG | AVIWYDGS | ARDPRGAT |
| (SEQ ID | | T (SEQ | (SEQ ID | NK (SEQ | LYYYYYG |
| NO: 33) | | ID NO: | NO: 35) | ID NO: | MDV (SEQ |
| | | 34) | | 36) | ID NO: 37) |

In some embodiments, the CTLA-4 antigen binding domain comprises an antibody. In some embodiments, the CTLA-4 antibody comprises a heavy chain and a light chain. In some embodiments, the CTLA4 antibody comprises a first heavy chain, a second heavy chain and two light chains (see, for example, FIG. 1A), and the sequences of the two chains are not the same. Exemplary CTLA-4 antibody sequences, including heavy chain, light chain, constant and variable regions are shown in Table 2, below.

TABLE 2
Sequences for antibodies specific to CTLA-4

| Name | Sequence |
|---|---|
| Light | EIVLTQSPGTLSLSPGERATLSCRASQSVGSSYLAWYQQKPGQAPRLLIY |
| Chain | GAFSRATGIPDRFSGSGSGTDFTLTISRLEPEDFAVYYCQQYGSSPWTFG |
| | QGTKVEIKRTVAAPSVFIFPPSDEQLKSGTASVVCLINNFYPREAKVQWK |
| | VDNALQSGNSQESVTEQDSKDSTYSLSSTLTLSKADYEKHKVYACEVTHQ |
| | GLSSPVTKSFNRGEC (SEQ ID NO: 9) |
| Light | GAGATTGTGCTGACCCAGAGCCCAGGTACACTGTCACTGTCCCCAGGCGA |
| Chain | GAGGGCTACTCTGTCTTGCCGGGCAAGCCAGTCTGTGGGTAGCTCTTACC |
| | TGGCCTGGTACCAGCAGAAGCCAGGACAGGCTCCACGACTGCTGATCTAC |
| | GGAGCATTCTCAAGAGCCACCGGGATTCCTGACCGCTTCAGTGGCTCAGG |
| | CTCCGGGACAGACTTCACCCTGACAATCTCCCGACTGGAGCCAGAAGACT |
| | TCGCCGTGTACTATTGCCAGCAGTATGGGTCCAGCCCCTGGACCTTTGGT |
| | CAGGGCACCAAGGTCGAGATCAAACGTACAGTGGCCGCTCCCTCCGTCTT |
| | CATTTTTCCCCCTAGCGACGAACAGCTGAAGTCTGGAACCGCTAGTGTGG |
| | TCTGTCTGCTGAACAATTTCTACCCTCGCGAAGCAAAGGTGCAGTGGAAA |
| | GTCGATAACGCCCTGCAGAGCGGCAATTCTCAGGAGAGTGTGACTGAACA |
| | GGACTCAAAGGATTCCACCTATAGCCTGTCTAGTACACTGACTCTGTCCA |
| | AAGCTGATTACGAGAAGCACAAAGTGTATGCATGTGAAGTCACTCATCAG |
| | GGGCTGTCTTCACCAGTCACCAAGTCCTTCAATCGTGGAGAATGC (SEQ |
| | ID NO: 17) |
| Heavy | QVQLVESGGGVVQPGRSLRLSCAASGFTFSSYTMHWVRQAPGKGLEWVTF |
| Chain | ISYDGNNKYYADSVKGRFTISRDNSKNTLYLQMNSLRAEDTAIYYCARTG |
| variable | WLGPFDYWGQGTLVTVSS (SEQ ID NO: 12) |
| region | |
| Heavy | CAGGTGCAGCTGGTGGAGAGCGGAGGAGGAGTGGTGCAGCCAGGCAGGTC |
| Chain | TCTGAGGCTGTCCTGCGCTGCTAGCGGCTTCACCTTTTCCAGCTACACAA |
| variable | TGCACTGGGTGAGGCAGGCTCCTGGCAAGGGCCTGGAGTGGGTGACCTTC |
| region | ATCTCCTATGACGGCAACAATAAATACTATGCTGATAGCGTGAAGGGCAG |
| | GTTCACCATCTCTCGCGACAACTCCAAGAATACACTGTACCTGCAGATGA |
| | ACTCTCTGAGAGCCGAGGACACCGCTATCTACTATTGCGCTAGGACAGGA |
| | TGGCTGGGACCTTTCGATTATTGGGGCCAGGGCACCCTGGTGACAGTGTC |
| | TTCC (SEQ ID NO: 28) |
| First or | ASTKGPSVFPLAPSSKSTSGGTAALGCLVKDYFPEPVTVSWNSGALTSGV |
| Second | HTFPAVLQSSGLYSLSSVVTVPSSSLGTQTYICNVNHKPSNTKVDKRVEP |
| Heavy | KSCDKTHTCPPCPAPELLGGPSVFLFPPKPKDTLMISRTPEVTCVVVDVS |
| Chain | HEDPEVKFNWYVDGVEVHNAKTKPREEQYNSTYRVVSVLTVLHQDWLNGK |
| constant | EYKCKVSNKALPAPIEKTISKAKGQPREPQVYTLPPSREEMTKNQVSLSC |
| region | AVKGFYPSDIAVEWESNGQPENNYKTTPPVLDSDGSFFLVSKLTVDKSRW |
| | QQGNVFSCSVMHEALHNHYTQKSLSLSPGK (SEQ ID NO: 13) |
| First or | GCGTCGACAAAGGGCCCCTCCGTGTTTCCTCTGGCTCCAAGCTCTAAGAG |
| Second | CACCTCTGGAGGAACAGCCGCTCTGGGATGTCTGGTGAAGGATTACTTCC |
| Heavy | CTGAGCCAGTGACCGTGAGCTGGAACTCTGGCGCCCTGACCTCTGGAGTG |
| Chain | CATACATTTCCCGCTGTGCTGCAGTCCAGCGGCCTGTATAGCCTGTCTTC |
| constant | CGTGGTGACCGTGCCTAGCTCTTCCCTGGGCACCCAGACATACATCTGCA |
| region | ACGTGAATCACAAGCCCTCCAATACAAAGGTGGACAAGAGAGTGGAGCCT |
| (DNA | AAGAGCTGTGATAAGACCCATACATGCCCACCATGTCCAGCTCCTGAGCT |
| sequence | GCTGGGAGGACCTTCCGTGTTCCTGTTTCCTCCAAAGCCAAAGGACACCC |
| encoding | TGATGATCTCTCGCACCCCTGAGGTGACATGCGTGGTGGTGGACGTGTCC |
| SEQ ID | CACGAGGATCCAGAGGTGAAGTTCAACTGGTACGTGGATGGCGTGGAGGT |
| NO: 13) | GCATAATGCTAAGACCAAGCCTAGGGAGGAGCAGTACAACAGCACCTATC |
| | GGGTGGTGTCTGTGCTGACAGTGCTGCACCAGGACTGGCTGAACGGCAAG |
| | GAGTACAAGTGCAAGGTGAGCAATAAGGCCCTGCCAGCTCCCATCGAGAA |
| | GACCATCTCTAAGGCCAAGGGCCAGCCCAGAGAGCCTCAGGTGTATACAC |
| | TGCCCCCTAGCCGCGAGGAGATGACCAAGAACCAGGTGTCTCTGTCATGT |
| | GCCGTGAAGGGCTTCTACCCATCTGACATCGCTGTGGAGTGGGAGTCCAA |
| | TGGCCAGCCCGAGAACAATTATAAGACCACACCACCCGTGCIGGACTCCG |
| | ATGGCTCATTCTTCCTGGTGTCCAAGCTGACCGTGGACAAGTCTAGATGG |
| | CAGCAGGGCAACGTGTTCTCCTGCTCCGTGATGCACGAGGCCCTGCACAA |
| | TCACTACACCCAGAAGTCCCIGTCTCTGTCCCCTGGAAAA (SEQ ID |
| | NO: 29) |
| First or | ASTKGPSVFPLAPSSKSTSGGTAALGCLVKDYFPEPVTVSWNSGALTSGV |
| Second | HTFPAVLQSSGLYSLSSVVTVPSSSLGTQTYICNVNHKPSNTKVDKRVEP |
| Heavy | KSCDKTHTCPPCPAPELLGGPSVFLFPPKPKDTLMISRTPEVTCVVVDVS |
| Chain | HEDPEVKFNWYVDGVEVHNAKTKPREEQYNSTYRVVSVLTVLHQDWLNGK |
| constant | EYKCKVSNKALPAPIEKTISKAKGQPREPQVYTIPPSREEMTKNQVSLWC |
| region | LVKGFYPSDIAVEWESNGQPENNYKTTPPVLDSDGSFFLYSKLTVDKSRW |
| | QQGNVFSCSVMHEALHNHYTOKSLSLSPGK (SEQ ID NO: 14) |
| First or | GCGTCGACAAAGGGCCCCTCCGTGTTTCCTCTGGCTCCAAGCTCTAAGAG |
| Second | CACCTCTGGAGGAACAGCCGCTCTGGGATGTCTGGTGAAGGATTACTTCC |
| Heavy | CTGAGCCAGTGACCGTGAGCTGGAACTCTGGCGCCCTGACCTCTGGAGTG |
| Chain | CATACATTTCCCGCTGTGCTGCAGTCCAGCGGCCTGTATAGCCTGTCTTC |
| constant | CGTGGTGACCGTGCCTAGCTCTTCCCTGGGCACCCAGACATACATCTGCA |
| region | ACGTGAATCACAAGCCCTCCAATACAAAGGTGGACAAGAGAGTGGAGCCT |
| (DNA | AAGAGCTGTGATAAGACCCATACATGCCCACCATGTCCAGCTCCTGAGCT |
| sequence | GCTGGGAGGACCTTCCGTGTTCCTGTTTCCTCCAAAGCCAAAGGACACCC |
| encoding | TGATGATCTCTCGCACCCCTGAGGTGACATGCGTGGTGGTGGACGTGTCC |
| SEQ ID | CACGAGGATCCAGAGGTGAAGTTCAACTGGTACGTGGATGGCGTGGAGGT |
| NO: 14) | GCATAATGCTAAGACCAAGCCTAGGGAGGAGCAGTACAACAGCACCTATC |
| | GGGTGGTGTCTGTGCTGACAGTGCTGCACCAGGACTGGCTGAACGGCAAG |
| | GAGTACAAGTGCAAGGTGAGCAATAAGGCCCTGCCAGCTCCCATCGAGAA |
| | GACCATCTCTAAGGCCAAGGGCCAGCCCAGAGAGCCTCAGGTGTATACAC |
| | TGCCCCCTAGCCGCGAGGAGATGACCAAGAACCAGGTGTCTCTGTGGTGT |
| | CTGGTGAAGGGCTTCTACCCATCTGACATCGCTGTGGAGTGGGAGTCCAA |
| | TGGCCAGCCCGAGAACAATTATAAGACCACACCACCCGTGCTGGACTCCG |
| | ATGGCAGCTTCTTTCTGTACTCCAAGCTGACCGTGGATAAGAGCAGGTGG |
| | CAGCAGGGCAACGTGTTTTCCTGCAGCGTGATGCACGAGGCCCTGCACAA |
| | TCATTATACACAGAAATCTCTGTCCCTGAGCCCAGGCAAG (SEQ ID |
| | NO: 30) |
| First or | QVQLVESGGGVVQPGRSLRLSCAASGFTFSSYTMHWVRQAPGKGLEWVTF |
| Second | ISYDGNNKYYADSVKGRFTISRDNSKNTLYLOMNSLRAEDTAIYYCARTG |
| Heavy | WLGPFDYWGQGTLVTVSSASTKGPSVFPLAPSSKSTSGGTAALGCLVKDY |
| Chain | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TIMISRTPEVTCVVVDVSHEDPEVKFNWYVDGVEVHNAKTKPREEQYNST |
| | YRVVSVLTVLHQDWINGKEYKCKVSNKALPAPIEKTISKAKGQPREPQVY |
| | TLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPGK |
| | (SEQ ID NO: 11) |
| First or | CAGGTGCAGCTGGTGGAGAGCGGAGGAGGAGTGGTGCAGCCAGGCAGGTC |
| Second | TCTGAGGCTGTCCTGCGCTGCTAGCGGCTTCACCTTTTCCAGCTACACAA |
| Heavy | TGCACTGGGTGAGGCAGGCTCCTGGCAAGGGCCTGGAGTGGGTGACCTTC |
| Chain | ATCTCCTATGACGGCAACAATAAATACTATGCTGATAGCGTGAAGGGCAG |
| (DNA | GTTCACCATCTCTCGCGACAACTCCAAGAATACACTGTACCTGCAGATGA |
| sequence | ACTCTCTGAGAGCCGAGGACACCGCTATCTACTATTGCGCTAGGACAGGA |
| encoding | TGGCTGGGACCTTTCGATTATTGGGGCCAGGGCACCCTGGTGACAGTGTC |
| SEQ ID | TTCCGCGTCGACAAAGGGCCCCTCCGTGTTTCCTCTGGCTCCAAGCTCTA |
| NO: 11) | AGAGCACCTCTGGAGGAACAGCCGCTCTGGGATGTCTGGTGAAGGATTAC |
| | TTCCCTGAGCCAGTGACCGTGAGCTGGAACTCTGGCGCCCTGACCTCTGG |
| | AGTGCATACATTTCCCGCTGTGCTGCAGTCCAGCGGCCTGTATAGCCTGT |
| | CTTCCGTGGTGACCGTGCCTAGCTCTTCCCTGGGCACCCAGACATACATC |
| | TGCAACGTGAATCACAAGCCCTCCAATACAAAGGTGGACAAGAGAGTGGA |
| | GCCTAAGAGCTGTGATAAGACCCATACATGCCCACCATGTCCAGCTCCTG |
| | AGCTGCTGGGAGGACCTTCCGTGTTCCTGTTTCCTCCAAAGCCAAAGGAC |
| | ACCCTGATGATCTCTCGCACCCCTGAGGTGACATGCGTGGTGGTGGACGT |
| | GTCCCACGAGGATCCAGAGGTGAAGTTCAACTGGTACGTGGATGGCGTGG |
| | AGGTGCATAATGCTAAGACCAAGCCTAGGGAGGAGCAGTACAACAGCACC |
| | TATCGGGTGGTGTCTGTGCTGACAGTGCTGCACCAGGACTGGCTGAACGG |
| | CAAGGAGTACAAGTGCAAGGIGAGCAATAAGGCCCTGCCAGCTCCCATCG |
| | AGAAGACCATCTCTAAGGCCAAGGGCCAGCCCAGAGAGCCTCAGGTGTAT |
| | ACACTGCCCCCTAGCCGCGAGGAGATGACCAAGAACCAGGTGTCTCTGTC |
| | ATGTGCCGTGAAGGGCTTCTACCCATCTGACATCGCTGTGGAGTGGGAGT |
| | CCAATGGCCAGCCCGAGAACAATTATAAGACCACACCACCCGTGCTGGAC |
| | TCCGATGGCTCATTCTTCCTGGTGTCCAAGCTGACCGTGGACAAGTCTAG |
| | ATGGCAGCAGGGCAACGTGTTCTCCTGCTCCGTGATGCACGAGGCCCTGC |
| | ACAATCACTACACCCAGAAGTCCCTGTCTCTGTCCCCTGGAAAA (SEQ |
| | ID NO: 31) |
| First or | QVQLVESGGGVVQPGRSLRLSCAASGFTFSSYTMHWVRQAPGKGLEWVTF |
| Second | ISYDGNNKYYADSVKGRFTISRDNSKNTLYLQMNSLRAEDTAIYYCARTG |
| Heavy | WLGPFDYWGQGTLVTVSSASTKGPSVFPLAPSSKSTSGGTAALGCLVKDY |
| Chain | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TIPPSREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK |
| | (SEQ ID NO: 10) |
| First or | CAGGTGCAGCTGGTGGAGAGCGGAGGAGGAGTGGTGCAGCCAGGCAGGAG |
| Second | CCTGAGGCTGTCTTGCGCTGCTTCCGGCTTCACCTTTTCCAGCTACACAA |
| Heavy | TGCACTGGGTGAGGCAGGCTCCTGGCAAGGGACTGGAGTGGGTGACCTTC |
| Chain | ATCTCTTATGACGGCAACAATAAATACTATGCTGATTCCGTGAAGGGCAG |
| (DNA | GTTCACCATCAGCCGCGACAACTCTAAGAATACACTGTACCTGCAGATGA |
| sequence | ACTCTCTGAGAGCCGAGGACACCGCTATCTACTATTGCGCCCGCACAGGA |
| encoding | TGGCTGGGACCCTTCGATTATTGGGGCCAGGGCACCCTGGTGACAGTGTC |
| SEQ ID | TTCCGCGTCGACAAAGGGCCCCTCCGTGTTTCCTCTGGCTCCAAGCTCTA |
| NO: 10) | AGAGCACCTCTGGAGGAACAGCCGCTCTGGGATGTCTGGTGAAGGATTAC |
| | TTCCCTGAGCCAGTGACCGTGAGCTGGAACTCTGGCGCCCTGACCTCTGG |
| | AGTGCATACATTTCCCGCTGTGCTGCAGTCCAGCGGCCTGTATAGCCTGT |
| | CTTCCGTGGTGACCGTGCCTAGCTCTTCCCTGGGCACCCAGACATACATC |
| | TGCAACGTGAATCACAAGCCCTCCAATACAAAGGTGGACAAGAGAGTGGA |
| | GCCTAAGAGCTGTGATAAGACCCATACATGCCCACCATGTCCAGCTCCTG |
| | AGCTGCTGGGAGGACCTTCCGTGTTCCTGTTTCCTCCAAAGCCAAAGGAC |
| | ACCCTGATGATCTCTCGCACCCCTGAGGTGACATGCGTGGTGGTGGACGT |
| | GTCCCACGAGGATCCAGAGGTGAAGTTCAACTGGTACGTGGATGGCGTGG |
| | AGGTGCATAATGCTAAGACCAAGCCTAGGGAGGAGCAGTACAACAGCACC |
| | TATCGGGTGGTGTCTGTGCTGACAGTGCTGCACCAGGACTGGCTGAACGG |
| | CAAGGAGTACAAGTGCAAGGTGAGCAATAAGGCCCTGCCAGCTCCCATCG |
| | AGAAGACCATCTCTAAGGCCAAGGGCCAGCCCAGAGAGCCTCAGGTGTAT |
| | ACACTGCCCCCTAGCCGCGAGGAGATGACCAAGAACCAGGTGTCTCTGTG |
| | GTGTCTGGTGAAGGGCTTCTACCCATCTGACATCGCTGTGGAGTGGGAGT |
| | CCAATGGCCAGCCCGAGAACAATTATAAGACCACACCACCCGTGCTGGAC |
| | TCCGATGGCAGCTTCTTTCTGTACTCCAAGCTGACCGTGGATAAGAGCAG |
| | GTGGCAGCAGGGCAACGTGTTTTCCTGCAGCGTGATGCACGAGGCCCTGC |
| | ACAATCATTATACACAGAAATCTCTGTCCCTGAGCCCAGGCAAG (SEQ |
| | ID NO: 19) |
| Heavy | QVQLVESGGGVVQPGRSLRLSCAASGFTFSSYTMHWVRQAPGKGLEWVTF |
| chain | ISYDGNNKYYADSVKGRFTISRDNSKNTLYLQMNSLRAEDTAIYYCARTG |
| fusion - | WLGPFDYWGQGTLVTVSSASTKGPSVFPLAPSSKSTSGGTAALGCLVKDY |
| hole | FPEPVTVSWNSGALTSGVHTFPAVLQSSGLYSLSSVVTVPSSSLGTQTYI |
| constant | CNVNHKPSNTKVDKRVEPKSCDKTHTCPPCPAPELLGGPSVFLFPPKPKD |
| region, | TLMISRTPEVTCVVVDVSHEDPEVKFNWYVDGVEVHNAKTKPREEQYNST |
| IL-15Ra | YRVVSVLTVLHQDWLNGKEYKCKVSNKALPAPIEKTISKAKGOPREPQVY |
| sushi | TLPPSREEMTKNQVSLSCAVKGFYPSDIAVEWESNGOPENNYKTTPPVLD |
| domain, | SDGSFFLVSKLTVDKSRWQQGNVFSCSVMHEALHNHYTQKSLSLSPGKGG |
| IL~15 | GGGSGGGGSGGGGSITCPPPMSVEHADIWVKSYSLYSRERYICNSGFKRK |
| domain | AGTSSLTECVLNKATNVAHWTTPSLKCIRDPALVHQRPAPPSTVGGGGSG |
| and G4S | GGGSGGGGSGGGGSNWVNVISDLKKIEDLIQSMHIDATLYTESDVHPSCK |
| linkers | VTAMKCFLLELQVISLESGDASIHDTVENLIILANNSLSSNGNVTESGCK |
| | ECEELEEKNIKEFLQSFVHIVQMFINTS (SEQ ID NO: 16) |
| Heavy | CAGGTGCAGCTGGTGGAGAGCGGAGGAGGAGTGGTGCAGCCAGGCAGGTC |
| chain | TCTGAGGCTGTCCTGCGCTGCTAGCGGCTTCACCTTTTCCAGCTACACAA |
| fusion - | TGCACTGGGTGAGGCAGGCTCCTGGCAAGGGCCTGGAGTGGGTGACCTTC |
| hole | ATCTCCTATGACGGCAACAATAAATACTATGCTGATAGCGTGAAGGGCAG |
| constant | GTTCACCATCTCTCGCGACAACTCCAAGAATACACTGTACCTGCAGATGA |
| region, | ACTCTCTGAGAGCCGAGGACACCGCTATCTACTATTGCGCTAGGACAGGA |
| IL-15Ra | TGGCTGGGACCTTTCGATTATTGGGGCCAGGGCACCCTGGTGACAGTGTC |
| sushi | TTCCGCGTCGACAAAGGGCCCCTCCGTGTTTCCTCTGGCTCCAAGCTCTA |
| domain, | AGAGCACCTCTGGAGGAACAGCCGCTCTGGGATGTCTGGTGAAGGATTAC |
| IL-15 | TTCCCTGAGCCAGTGACCGTGAGCTGGAACTCTGGCGCCCTGACCTCTGG |
| domain | AGTGCATACATTTCCCGCTGTGCTGCAGTCCAGCGGCCTGTATAGCCTGT |
| and G4S | CTTCCGTGGTGACCGTGCCTAGCTCTTCCCTGGGCACCCAGACATACATC |
| linkers | TGCAACGTGAATCACAAGCCCTCCAATACAAAGGTGGACAAGAGAGTGGA |
| | GCCTAAGAGCTGTGATAAGACCCATACATGCCCACCATGTCCAGCTCCTG |
| | AGCTGCTGGGAGGACCTTCCGTGTTCCTGTTTCCTCCAAAGCCAAAGGAC |
| | ACCCTGATGATCTCTCGCACCCCTGAGGTGACATGCGTGGTGGTGGACGT |
| | GTCCCACGAGGATCCAGAGGTGAAGTTCAACTGGTACGTGGATGGCGTGG |
| | AGGTGCATAATGCTAAGACCAAGCCTAGGGAGGAGCAGTACAACAGCACC |
| | TATCGGGTGGTGTCTGTGCTGACAGTGCTGCACCAGGACTGGCTGAACGG |
| | CAAGGAGTACAAGTGCAAGGTGAGCAATAAGGCCCTGCCAGCTCCCATCG |
| | AGAAGACCATCTCTAAGGCCAAGGGCCAGCCCAGAGAGCCTCAGGTGTAT |
| | ACACTGCCCCCTAGCCGCGAGGAGATGACCAAGAACCAGGTGTCTCTGTC |
| | ATGTGCCGTGAAGGGCTICTACCCATCTGACATCGCTGTGGAGTGGGAGT |
| | CCAATGGCCAGCCCGAGAACAATTATAAGACCACACCACCCGTGCTGGAC |
| | TCCGATGGCTCATTCTTCCTGGTGTCCAAGCTGACCGTGGACAAGTCTAG |
| | ATGGCAGCAGGGCAACGTGTTCTCCTGCTCCGTGATGCACGAGGCCCTGC |
| | ACAATCACTACACCCAGAAGTCCCTGTCTCTGTCCCCTGGAAAAGGCGGA |
| | GGCGGAGGATCTGGTGGTGGTGGATCTGGCGGCGGAGGCTCTATTACATG |
| | CCCTCCTCCAATGTCCGTGGAACACGCCGACATCTGGGTCAAGTCCTACA |
| | GCCTGTACTCCAGAGAGCGGTACATCTGCAACTCCGGCTTCAAGAGAAAG |
| | GCCGGCACCTCTAGCCTGACCGAGTGCGTGCTGAACAAGGCCACCAATGT |
| | GGCCCACTGGACCACACCTAGCCTGAAGTGCATCAGGGACCCCGCTCTGG |
| | TTCATCAGAGGCCTGCTCCTCCATCTACCGTTGGTGGCGGAGGTAGCGGT |
| | GGTGGCGGTAGCGGAGGCGGTGGTTCTGGCGGAGGCGGTTCTAATTGGGT |
| | CAACGTGATCTCCGACCTGAAGAAGATCGAGGACCTGATCCAGTCCATGC |
| | ACATCGACGCTACCCTGTACACCGAGTCCGACGTGCACCCTTCCTGTAAA |
| | GTGACCGCCATGAAGTGCTTTCTGCTGGAACTGCAAGTGATCTCCCTGGA |
| | ATCCGGCGACGCCTCTATCCACGACACCGTGGAAAACCTGATCATCCTGG |
| | CCAACAACTCCCTGTCCTCCAACGGCAACGTGACCGAGTCTGGCTGCAAA |
| | GAGTGCGAGGAACTGGAAGAGAAGAACATCAAAGAGTTCCTCCAGTCCTT |
| | CGTGCACATCGTGCAGATGTTCATCAACACCAGC (SEQ ID NO: 18) |
| Ipilimumab | QVQLVESGGGVVQPGRSLRLSCAASGFTFSSYTMHWVRQAPGKGLEWVTF |
| Heavy | ISYDGNNKYYADSVKGRFTISRDNSKNTLYLQMNSLRAEDTAIYYCARTG |
| Chain | WLGPFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TIPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| | (SEQ ID NO: 8) |

In some embodiments, the CTLA-4 antibody comprises a light chain sequence comprising a sequence of SEQ ID NO: 9, or a sequence having at least 80%, at least 85%, at least 90%, at least 95% or at least 99% identity thereto. In some embodiments, the CTLA-4 antibody comprises a light chain sequence comprising, or consisting essentially of, SEQ ID NO: 9.

In some embodiments, the CTLA-4 antibody comprises one or more heavy chain sequences comprising a heavy chain variable region sequence of SEQ ID NO: 12, or a sequence having at least 80%, at least 85%, at least 90%, at least 95% or at least 99% identity thereto. In some embodiments, the CTLA-4 antibody comprises one or more heavy chain sequences comprising a heavy chain variable region sequence of SEQ ID NO: 12, or a sequence having at least 90%, at least 95% or at least 99% identity thereto. In some embodiments, the heavy chain variable region sequence comprises, or consists essentially of, SEQ ID NO: 12. In some embodiments, the heavy chain variable regions is encoded by a polynucleotide comprising a sequence of SEQ ID NO: 28.

In some embodiments, the CTLA-4 antibody comprises a first heavy chain and second heavy chain, and the sequences of the two heavy chains are not identical. In some embodiments, both the first and second heavy chains comprise heavy chain variable region sequence of SEQ ID NO: 12, or a sequence having at least 80%, at least 85%, at least 90%, at least 95% or at least 99% identity thereto. In some embodiments, both the first and second heavy chains comprise heavy chain variable region sequence comprising, or consisting essentially of, SEQ TD NO: 12.

Heavy chain constant regions of the antibodies described herein may be engineered to preferentially form a heterodimer of two different heavy chains. A non-limiting example of such engineering is “knob into hole” technology which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of both heavy chains containing these two CH3 domains. One of the two CH3 domains (of the two heavy chains) can be the “knob”, while the other is the “hole”. For example, the Ipilimumab heavy chain (SEQ ID NO: 8) can be engineered with T367S, L369A and Y408V mutations to generate a “hole” heavy chain, and with a T367W mutation to generate a “knob” heavy chain. Knob and hole heavy chains carrying these substitutions will preferentially (i.e., with greater frequency) form a knob/hole heterodimer, instead of knob/knob or hole/hole homodimer.

In some embodiments, the first and second heavy chains differ by at least one amino acid in the constant region. For example, the first and second heavy chains can have, 1, 2, 3, 4, or 5 amino acid differences in the heavy chain constant region. In some embodiments, the first and second heavy chains differ by 3 amino acids in the constant regions. For example, the first and second heavy chains may differ at positions 249, 251 290 or any combination thereof of SEQ ID NO: 13 or SEQ ID NO: 14. In some embodiments, the first heavy chain comprises an S at position 249, an A at position 251 and a V at position 290 of SEQ ID NO: 13 or SEQ ID NO: 14, while the second heavy chain comprises a W at position 249, an L at position 251 and a Y at position 290 of SEQ ID NO: 13 or 14.

In some embodiments, the first and second heavy chains may differ at positions 350, 355, 367, 369, 408 or any combination thereof of SEQ ID NOS: 10 or 11.

In some embodiments, the first heavy chain comprises an S at position 367, an A at position 369 and a T at position 408 relative to SEQ ID NO: 10 or 11, and the second heavy chain comprises a W at position 367 relative to SEQ ID NO: 10 or 11. In some embodiments, the first heavy chain comprises an S at position 367, an A at position 369 and a V at position 408 relative to SEQ ID NOS: 10 or 11, and the second heavy chain comprises a W at position 367 relative to SEQ ID NOS: 10 or 11. Optionally, the first heavy chain comprises a C at position 350 relative to SEQ ID NO: 10 or 11, and the second heavy chain comprises a C at position 355 relative to SEQ ID NO: 10 or 11.

In some embodiments, the first heavy chain comprises a T at position 408 relative to SEQ ID NOS: 10 or 11, and the second heavy chain comprises a W at position 367 relative to SEQ ID NOS: 10 or 11.

In any of the foregoing embodiments, the first and/or second heavy chain can comprise an F at position 235, an A at position 250 and an A at position 435 relative to SEQ ID NOS: 10 or 11.

In some embodiments, the first heavy chain comprises a sequence of SEQ ID NO: 13, or a sequence having at least 80%, at least 85%, at least 90%, at least 95% or at least 99% identity thereto, and an A at position 251 and a V at position 290 of SEQ ID NO: 13. In some embodiments, the first heavy chain comprises a sequence of SEQ ID NO: 13.

In some embodiments, the second heavy chain comprises a sequence of SEQ ID NO: 14, or a sequence having at least 80%, at least 85%, at least 90%, at least 95% or at least 99% identity thereto, and a W at position 249, an L at position 251 and a Y at position 300 of SEQ ID NO: 14. In some embodiments, the second heavy chain comprises a sequence of SEQ ID NO: 14. In some embodiments, the first heavy chain comprises a sequence of SEQ ID NO: 13, and the second heavy chain comprises a sequence of SEQ ID NO: 14.

In some embodiments, the first heavy chain comprises a sequence of SEQ ID NO: 11, or a sequence having at least 80%, at least 85%, at least 90%, at least 95% or at least 99% identity thereto. In some embodiments, the first heavy chain comprises a sequence of SEQ ID NO: 11, or a sequence having at least 80%, at least 85%, at least 90%, at least 95% or at least 99% identity thereto, and a S at position 367, an A at position 369 and a V at position 408 of SEQ ID NO: 11. In some embodiments, the first heavy chain comprises, or consists essentially of, SEQ ID NO: 11. In some embodiments, the second heavy chain comprises a sequence of SEQ ID NO: 10, or a sequence having at least 80%, at least 85%, at least 90%, at least 95% or at least 99% identity thereto. In some embodiments, the first heavy chain comprises a sequence of SEQ ID NO: 10, or a sequence having at least 80%, at least 85%, at least 90%, at least 95% or at least 99% identity thereto, and a W at position 367 of SEQ ID NO: 10. In some embodiments, the second heavy chain comprises, or consists essentially of, SEQ ID NO: 10. In some embodiments, the first heavy chain comprises, or consists essentially of, SEQ ID NO: 11, and the second heavy chain comprises, or consists essentially of, SEQ ID NO: 10.

### Linkers

In some embodiments of the fusion proteins of the disclosure, the fusion protein comprises one or more linkers. For example, in a fusion protein with the IL-15 domain and IL-15Ra sushi domains fused to the heavy chain of an anti-CTLA-4 antibody, one or more of the heavy chain, the IL-15 domain and the IL-15Ra domain can be separated by a linker.

The term “linker” is art-recognized and refers to a molecule (including but not limited to unmodified or modified nucleic acids or amino acids) or group of molecules (for example, 2 or more, e.g., 2, 3, 4, 5, 6, 7, 8, 9, 10, 15, 20, 25, 30, 35, 40, 45, 50, 55, 60, 65, 70, 75, 80, 85, 90, 95, 100 or more) connecting two compounds, such as two polypeptides. The linker may be comprised of a single linking molecule or may comprise a linking molecule and at least one spacer molecule, intended to separate the linking molecule and a compound by a specific distance.

In some embodiments, the linker comprises a Glycine-Serine (GS) linker. Exemplary GS linkers include, but are not limited to GGGS (SEQ ID NO: 23), GGGGS (SEQ ID NO: 24), GGGGSGGGGS (SEQ ID NO: 25), GGGGSGGGGSGGGGS (SEQ ID NO: 26), and GGGGSGGGGSGGGGSGGGGS (SEQ ID NO: 15).

In some embodiments, the CTLA-4 antigen binding domain, and the IL-15Ra domain are separated by a linker. In some embodiments, for example those embodiments where the CTLA-4 antigen binding domain is an antibody, the first or second heavy chain of the antibody and the IL-15Ra domain are separated by a linker. In some embodiments, the linker comprises GGGS (SEQ ID NO: 23), GGGGS (SEQ ID NO: 24), GGGGSGGGGS (SEQ ID NO: 25), GGGGSGGGGSGGGGS (SEQ ID NO: 26), or GGGGSGGGGSGGGGSGGGGS (SEQ ID NO: 15). In some embodiments, the linker comprises GGGGGSGGGGSGGGGS (SEQ ID NO: 26). In some embodiments, the sequence encoding the linker comprises a sequence of GGCGGAGGCGGAGGATCTGGTGGTGGTGGATCTGGCGGCGGAGGCTCT (SEQ ID NO: 27). In some embodiments, the IL-15Ra sushi domain and the IL-15 domain are separated by a linker. In some embodiments, the linker comprises GGGS (SEQ ID NO: 23), GGGGS (SEQ ID NO: 24), GGGGSGGGGS (SEQ ID NO: 25), GGGGSGGGGSGGGGS (SEQ ID NO: 26), or GGGGSGGGGSGGGGSGGGGS (SEQ ID NO: 15). In some embodiments, the linker comprises GGGGSGGGGSGGGGSGGGGS (SEQ ID NO: 15). In some embodiments, the sequence encoding the linker comprises a sequence of

(SEQ ID NO: 32)
GGTGGCGGAGGTAGCGGTGGTGGCGGTAGCGGAGGCGGTGGTTCTGGCGG
AGGCGGTTCT.

In some embodiments of the recombinant fusion protein described herein, the antigen binding domain specific to CTLA-4 comprises an antibody. In some embodiments, the antibody comprises two heavy chains, and two light chains, as seen in FIG. 1A. In some embodiments, the sequences of the two heavy chains (the first and second heavy chain, as referred to herein), are not identical. For example, the first heavy chain comprises one or more modifications to the constant region to produce a “hole” variant, and the second heavy chain comprises one or more modifications to the constant region to produce a “knob” variant, or vice versa. In some embodiments, the hole and knob variants preferentially associate to form a heterodimer. In some embodiments, the first heavy chain comprises a constant region sequence of SEQ ID NO: 13, and the second heavy chain comprises a constant region sequence of SEQ ID NO: 14. In alternative embodiments, the first heavy chain comprises a sequence of SEQ ID NO: 14, and the second heavy chain comprises a sequence of SEQ ID NO: 13.

### Polypeptides

The disclosure provides a recombinant fusion protein, comprising: (a) a first polypeptide comprising, from N- to C-terminus, sequences of a first CTLA-4 antibody heavy chain, an IL-15Ra sushi domain and an IL-15 domain; (b) a second polypeptide comprising a sequence of a second CTLA-4 heavy chain; and (c) two additional polypeptides comprising a sequence of a CTLA-4 antibody light chain.

In some embodiments, the first polypeptide comprises, from N- to C-terminus, the sequences of a heavy chain of an anti-CTLA-4 antibody, a first linker, an IL-15Ra sushi domain, a second linker, and an IL-15 domain. In some embodiments, the recombinant fusion protein comprises a second polypeptide comprising the sequence of a second heavy chain of an anti-CTLA-4 antibody whose sequence not identical to the first heavy chain (e.g., the first heavy chain comprises a hole variant, and the second heavy chain comprises a knob variant, or vice versa). In some embodiments, the first and second polypeptides preferentially form a heterodimer. In some embodiments, the heavy chain and the 1L-15Ra sushi domain are separated by a first linker. In some embodiments, the IL-15Ra sushi domain and IL-15 domain are separated by a second linker. In some embodiments, the first and/or second linkers are Glycine-Serine linkers.

In some embodiments, the first polypeptide comprises, from N- to C-terminus, sequences of an anti-CTLA heavy chain of SEQ ID NO: 11, a first linker of SEQ ID NO: 26, an IL-15Ra sushi domain of SEQ ID NO: 2, a second linker of SEQ ID NO: 15, and an IL-15 domain of SEQ ID NO: 1, or sequences having at least 80%, at least 85%, at least 90%, at least 95% or at least 99% identity thereto. In some embodiments, the first polypeptide comprises, from N- to C-terminus, sequences of an anti-CTLA heavy chain of SEQ ID NO: 11, a first linker of SEQ ID NO: 26, an IL-15Ra sushi domain of SEQ ID NO: 2, a second linker of SEQ ID NO: 15, and an IL-15 domain of SEQ ID NO: 1. In some embodiments, the first polypeptide comprises a sequence of SEQ ID NO: 16, or a sequence having at least 80%, at least 85%, at least 90%, at least 95% or at least 99% identity thereto. In some embodiments, the first polypeptide comprises, or consists essentially of, a sequence of SEQ ID NO: 16. In some embodiments, the first polypeptide is encoded by a polynucleotide comprising a sequence of SEQ ID NO: 18, or a sequence having at least 80%, at least 85%, at least 90%, at least 95% or at least 99% identity thereto. In some embodiments, the first polypeptide is encoded by a polynucleotide comprising a sequence of SEQ ID NO: 18.

In some embodiments, the second polypeptide comprising the second heavy chain of the anti-CTLA-4 antibody does not comprise a fusion of the second heavy chain to any additional heterologous domains, such as the IL-15Ra sushi domain or IL-15 domain. In some embodiments, the second heavy chain comprises, or consists essentially of, a sequence of SEQ ID NO: 10, or a sequence having at least 80%, at least 85%, at least 90%, at least 95% or at least 99% identity thereto. In some embodiments, the second heavy chain comprises, or consists essentially of, a sequence of SEQ ID NO: 10. In some embodiments, the second polypeptide is encoded by a polynucleotide comprising a sequence of SEQ 1D NO: 19, or a sequence having at least 80%, at least 85%, at least 90%, at least 95% or at least 99% identity thereto. In some embodiments, the second polypeptide is encoded by a polynucleotide comprising a sequence of SEQ ID NO: 19.

In some embodiments, the recombinant fusion protein further comprises two additional polypeptides comprising a sequence of a light chain of an anti-CTLA-4 antibody. In some embodiments, the two additional polypeptides comprising the anti-CTLA-4 light chains comprise, or consist essentially of, a sequence of SEQ ID NO: 9, or a sequence having at least 80%, at least 85%, at least 90%, at least 95%, or at least 99% identity thereto. In some embodiments, the two additional polypeptides comprising the anti-CTLA-4 light chains comprise, or consist essentially of, a sequence of SEQ ID NO: 9. In some embodiments, the anti-CTLA 4 antibody light chain sequences of the two additional polypeptides are encoded by a polynucleotide comprising a sequence of SEQ ID NO: 17, or a sequence having at least 80%, at least 85%, at least 90%, at least 95% or at least 99% identity thereto. In some embodiments, the anti-CTLA 4 antibody light chain sequences of the two additional polypeptides are encoded by a polynucleotide comprising a sequence of SEQ ID NO: 17.

In some embodiments, recombinant fusion protein comprises a first polypeptide comprising a sequence of SEQ ID NO: 16, a second polypeptide comprising a sequence of SEQ ID NO: 10, and two additional polypeptides comprising a sequence of SEQ ID NO: 9.

### Polynucleotides and Vectors

The disclosure provides polynucleotides encoding the recombinant fusion proteins described herein.

The disclosure provides a first polynucleotide comprising a sequence encoding a first polypeptide comprising, from N- to C-terminus, a first heavy chain of an anti-CTLA heavy chain of SEQ ID NO: 11, a first linker of SEQ ID NO: 26, an IL-15Ra sushi domain of SEQ ID NO: 2, a second linker of SEQ ID NO: 15, and an IL-15 domain of SEQ ID NO: 1. In some embodiments, the first polynucleotide comprises a sequence of SEQ ID NO: 16, or a sequence having at least 80%, at least 85%, at least 90%, at least 95% or at least 99% identity thereto. In some embodiments, the first polynucleotide comprises a sequence of SEQ ID NO: 16.

The disclosure provides a second polynucleotide comprising a sequence encoding a second polypeptide comprising second heavy chain of an anti-CTLA-4 antibody of SEQ ID NO: 10. In some embodiments, the second heavy chain is not fused to additional heterologous domains, such as the IL-15Ra sushi domain or IL-15 domain. In some embodiments, the second heavy chain comprises, or consists essentially of, a sequence of SEQ ID NO: 10, and the second polynucleotide comprises a sequence of SEQ ID NO: 19, or a sequence having at least 80%, at least 85%, at least 90%, at least 95% or at least 99% identity thereto. In some embodiments, the second polynucleotide comprises a sequence of SEQ ID NO: 19.

The disclosure provides a third polynucleotide comprising a sequence encoding a third polynucleotide encoding a light chain of an anti-CTLA-4 antibody. In some embodiments, the anti-CTLA-4 antibody light chain comprises, or consist essentially of, a sequence of SEQ ID NO: 9, and the third polynucleotide comprises a sequence of SEQ ID NO: 17, or a sequence having at least 80%, at least 85%, at least 90%, at least 95% or at least 99% identity thereto. In some embodiments, the third polynucleotide comprises a sequence of SEQ ID NO: 17.

In some embodiments, the polynucleotide sequences encoding each of the first, second and third polypeptides are operably linked to one or more promoters. For example, sequences of two or more of the polypeptides can be operably linked (under the control of) the same promoter, and separated by one or more elements that produce separate polypeptides, such as self-cleaving polypeptides, internal ribosome entry sites, and the like.

In alternative embodiments, the sequences first, second and third polynucleotides encoding the first second and third polypeptides comprising the first heavy chain, second heavy chain, and light chain are under the control of three separate promoters. For example, each of the first, second and third polynucleotides may be cloned into a separate expression vector, each vector comprising its own promoter and/or regulatory sequences. In some embodiments, the promoters operably linked to each of the first, second and third polynucleotides are the same. In some embodiments, the promoters operably linked to each of the first, second and third polynucleotides are not the same.

In some embodiments, one or more of the first, second and third polynucleotides encoding the first, second and third polypeptides comprising the first heavy chain, second heavy chain, and light chain are part of a single, contiguous polynucleotide molecule.

In alternative embodiments, the first, second and third polypeptides comprising the first heavy chain, second heavy chain, and light chain are each encoded by polynucleotide sequences on different, non-contiguous polynucleotide molecules.

In some embodiments, polynucleotides of the present invention are prepared using PCR techniques using procedures and methods known to one skilled in the art. In some embodiments, the procedure involves the ligation of two different DNA sequences (See, for example, “Current Protocols in Molecular Biology”, eds. Ausubel et al., John Wiley & Sons, 1992).

A polynucleotide sequence is “operably linked” when it is placed into a functional relationship with another polynucleotide sequence. For example, a polynucleotide presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the polynucleotide sequences being linked are contiguous, and, in the case of a secretory leader, contiguous and in reading frame. However, enhancers are optionally contiguous. Linking can be accomplished, for example, by ligation at convenient restriction sites. If such sites do not exist, synthetic oligonucleotide adaptors, linkers or other methods known in the art can be used. In another embodiment, the “operably linked” also refers to the functional pairing of distinct amino acid sequences, peptides or proteins, as in the combination of the anti-CTLA-4 antibody and IL-15Ra sushi domain and IL-15 described herein via a linker sequence also described herein.

The disclosure provides vectors comprising the polynucleotides comprising the recombinant fusion proteins described herein.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or transmembrane. The term “intracellular” means something that is inside a cell. The term “extracellular” means something that is outside a cell. The term transmembrane means something that has an extracellular domain outside the cell, a portion embedded in the cell membrane and an intracellular domain inside the cell.

In some embodiments polynucleotides of the present invention are inserted into expression vectors (i.e., a nucleic acid construct) to enable expression of the recombinant fusion proteins and polypeptides described herein.

In some embodiment, the expression vector of the present invention includes additional sequences which render this vector suitable for replication and integration in prokaryotes. In some embodiments, the expression vector of the present invention includes additional sequences which render this vector suitable for replication and integration in eukaryotes. In some embodiments, the expression vector of the present invention includes a shuttle vector which renders this vector suitable for replication and integration in both prokaryotes and eukaryotes. For example, such vectors may include selectable markers appropriate for both eukaryotic and prokaryotic cells. Suitable markers will be apparent to persons of ordinary skill in the art.

In some embodiments, cloning vectors comprise transcription and translation initiation sequences (e.g., promoters, enhancer) and transcription and translation terminators (e.g., polyadenylation signals) to enhance expression of polypeptides expressed therefrom. Suitable translation terminators include, but are not limited, to bovine growth hormone polyadenylation signals (BGH polyA) and the like. Suitable promoters will be apparent to persons of the ordinary skill in the art, and include the CMV promoter, actin promoter and the like.

In some embodiments, the expression vectors of the present invention can further include additional polynucleotide sequences that allow, for example, the translation of several proteins from a single mRNA such as an internal ribosome entry site (IRES) and sequences for genomic integration of the promoter-chimeric polypeptide.

In some embodiments, the expression vectors of the present invention include elements that increase the expression of the recombinant fusion proteins of the invention. Such features include, but are not limited to, choice of promoter and polyadenylation. In some embodiments, the polyadenylation sequence is a bovine growth hormone (BGH) polyadenylation sequence. In some embodiments, the promoter comprises a constitutively active promoter. In some embodiments, the promoter comprises a cytomegalovirus promoter (pCMV). Promoters can, in some embodiments, be combined with additional elements to promote expression of the recombinant proteins of the disclosure, such as introns (e.g., rabbit beta globin intron, EF1a intron and the like) and enhancer elements (CMV immediate early enhancer, SV40 enhancer, EF1a enhancer, adenoviral major late protein enhancer, and the like).

Exemplary mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Barr virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, for example in bacterial systems used to express the recombinant fusion proteins of the present invention, a number of expression vectors can be advantageously selected depending upon the use intended for the protein expressed. In some embodiments, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of E. coli expression vectors [Studier et al., Methods in Enzymol. 185:60-89 (1990)].

In some embodiments, yeast expression systems are used to express the recombinant fusion proteins of the disclosure. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In some embodiments, mammalian cell expression systems are used to express the recombinant fusion proteins of the disclosure. The mammalian cells can be, for example Chinese Hamster Ovary (CHO) cells or derivatives thereof, and the vector is a vector suitable for expression of the recombinant fusion protein in CHO cells.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

### Methods of Manufacture

The disclosure provides methods of making the recombinant fusion proteins comprising the CTLA-4 antigen binding domain, IL-15Ra sushi domain, and IL-15 domain, comprising: (a) contacting a plurality of cells with polynucleotides or vectors encoding the recombinant fusion protein; (b) expressing the recombinant fusion protein by the plurality of cells; and (c) purifying the recombinant fusion protein.

A variety of prokaryotic or eukaryotic cells can be used as host-expression systems to express the recombinant fusion proteins of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence.

In some embodiments, the plurality of cells comprises eukaryotic cells. In some embodiments, the eukaryotic cells are mammalian cells. Mammalian cells suitable for expression of recombinant fusion proteins include CHO cells, PER.C6 cells, murine NS0 cells, and HEK293 cells. Selection of a suitable cell line will be apparent to persons of ordinary skill in the art.

In some embodiments, the plurality of cells comprises prokaryotic cells, for example E. coli cells.

In some embodiments, contacting the plurality of cells with the polynucleotides or vectors encoding the recombinant fusion protein comprises transfection.

The term “transfection” means the introduction of a foreign nucleic acid into a cell using recombinant DNA technology. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

In some embodiments, contacting the plurality of cells with the polynucleotides or vectors encoding the recombinant fusion protein comprises transduction. The term “transduction” means the introduction of a foreign nucleic acid into a cell using a viral vector, such as a lentiviral vector.

In some embodiments, non-bacterial expression systems are used (e.g., mammalian expression systems such as CHO cells) to express the polypeptide of the present invention. In some embodiments, the expression vector used to express polynucleotides of the present invention in mammalian cells comprises a CMV promoter and a neomycin resistance gene. In alternative embodiments, the expression vector used to express polynucleotides of the present invention in mammalian cells comprises a glutamine synthase marker (GS) under control of an SV40 promoter.

In some embodiment, various methods can be used to introduce the expression vector encoding the recombinant fusion protein of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors; A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In some embodiments, transformed cells are cultured under effective conditions, which allow for the expression of high amounts of recombinant fusion protein or polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

For example, appropriate media for the culture of eukaryotic cells includes, but is not limited to, but are not limited to Iscove's Modified Dulbecco's Medium, RPMI 1640, Minimal Essential Medium-alpha (MEM-alpha), Dulbecco's Modification of Eagle's Medium (DMEM), Grace's Complete Insect Medium, Ham's F-10 or F-12 with L-Glutamine, Schneider's Insect Medium, or any other media known to one skilled in the art. Additionally, culture media as described herein include, but are not limited to, chemically defined media, hydrolysate-containing media, and simple media. Choice of appropriate media and cell culture conditions for a particular cell type will be apparent to the person of ordinary skill in the art.

In some embodiments, depending on the vector and host system used for production, resultant polypeptides of the present invention either remain within the recombinant cell, secreted into the fermentation medium, secreted into a space between two cellular membranes, such as the periplasmic space in E. coli; or retained on the outer surface of a cell or viral membrane.

In some embodiments, following a predetermined time in culture, the recombinant fusion protein or polypeptide is recovered.

In one embodiment, the phrase “recovering the recombinant polypeptide” used herein refers to collecting the whole fermentation medium containing the polypeptide and need not imply additional steps of separation or purification.

In some embodiments, polypeptides of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In some embodiments, to facilitate recovery, the expressed coding sequence can be engineered to encode the polypeptide of the present invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In some embodiments, the polypeptide of the present invention is retrieved in “substantially pure” form. The phrase “substantially pure” refers to a purity that allows for the effective use of the protein in the applications described herein.

In some embodiments, the polypeptides of the present invention can also be synthesized using in vitro expression systems. In one embodiment, in vitro synthesis methods are well known in the art and the components of the system are commercially available.

In some embodiments, the polypeptides are synthesized and purified; and their therapeutic efficacy is assayed in vivo or in vitro.

### Pharmaceutical Compositions

The disclosure provides pharmaceutical compositions, comprising recombinant fusion proteins comprising a CTLA-4 antigen binding domain, IL-15Ra sushi domain and IL-15 domain, and a pharmaceutically acceptable carrier, diluent or excipient.

As used herein, “pharmaceutical carrier” includes any and all solvents, dispersion media, coatings, antibacterial and antifungal agents, isotonic and absorption delaying agents, and the like that are physiologically compatible. Carrier materials are non-toxic and do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. Preferably, the carrier is suitable for intravenous, intramuscular, subcutaneous, parenteral, spinal or epidermal administration (e.g. by injection or infusion).

Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The pharmaceutical compositions may be present in a form known in the art and acceptable for therapeutic uses. In some embodiments, pharmaceutical compositions of the invention is a liquid formulation. In other embodiments, pharmaceutical compositions of the invention are lyophilized. In further embodiments, pharmaceutical compositions of the invention are reconstituted liquid formulations. In some embodiments, a liquid formulation of the invention is an aqueous formulation. In other embodiments, the liquid formulation is non-aqueous.

Compositions comprising the recombinant fusion proteins of the present disclosure can be formulated for administration by a variety of methods known in the art. As will be appreciated by the person of ordinary skill in the art, the route and/or mode of administration will vary depending upon the desired results. To administer a composition of the disclosure by certain routes of administration, it may be necessary to co-administer the composition with, a material to prevent its inactivation. For example, the recombinant fusion proteins may be administered to a subject in an appropriate carrier, for example, liposomes, or a diluent.

In some embodiments, preparations for administration to subjects include sterile aqueous or non-aqueous solutions, suspensions, and emulsions. Some embodiments include non-aqueous solvents such as propylene glycol, polyethylene glycol, vegetable oils (e.g., olive oils), organic esters (e.g., ethyl oleate) and other solvents known to those of skill in the art. Physiologically acceptable carriers (or excipients) are optionally used in certain embodiments of the invention. Examples of such include, e.g., saline, PBS, Ringer's solution, lactated Ringer's solution, etc. Additionally, preservatives and additives are optionally added to the compositions to help ensure stability and sterility. For example, antibiotics and other bacteriocides, antioxidants, chelating agents, and the like are all optionally present in various embodiments of the compositions herein.

Regardless of the route of administration selected, the compositions of the disclosure, which may be used in a suitable hydrated form, and/or the pharmaceutical compositions of the present invention, are formulated into pharmaceutically acceptable dosage forms by conventional methods known to those of ordinary skill in the art.

The recombinant fusion protein, or pharmaceutical composition comprising the same are optionally administered to subjects in need of treatment (either therapeutically or prophylactically) in any appropriate sterile pharmaceutical carrier. Such pharmaceutical carrier acts to maintain the solubility and action of the fusion protein.

In some embodiments, compositions for use in the methods disclosed herein comprise solutions or emulsions, which in some embodiments are aqueous solutions or emulsions comprising a safe and effective amount of the compounds disclosed herein and optionally, other compounds, intended for various routes of administration.

The composition must be sterile and fluid to the extent that the composition is deliverable by syringe. In addition to water, the carrier preferably is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Actual dosage levels of the active ingredients in the pharmaceutical compositions of the present invention may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts.

### Therapeutic Methods

The disclosure provides a method of treating a disease or disorder in a subject in need thereof, the method comprising administering a therapeutically effective amount of the recombinant fusion proteins or the pharmaceutical composition comprising the recombinant fusions protein disclosed herein.

In some embodiments of the methods describe herein, the recombinant fusion protein or pharmaceutical composition comprising same inhibits the activity of CTLA-4 on an immune cell of the subject. For example, the CTLA-4 antigen binding domain of the recombinant fusion protein acts as a CTLA-4 antagonist.

In some embodiments, the recombinant fusion protein or pharmaceutical composition increases the activity of an Interleukin 2/Interleukin 15 receptor beta (IL-2Rb)/common gamma chain (IL-2RG) receptor complex an immune cell. For example, the combined IL-15Ra sushi domain and IL-15 domain bind to and activate that IL-2/IL-15Rb/common gamma chain receptor complex. The immune cell can be an immune cell of the subject, or an immune cell administered to the subject, for example as part of an adoptive cell therapy.

In some embodiments, the recombinant fusion protein or pharmaceutical composition promotes an activity in an immune cell. In some embodiments, the activity comprises activation, proliferation or a combination thereof. In some embodiments, the immune cell is a T cell, B cell or NK cell. In some embodiments, the T cell is a CD8+ T cell.

In some embodiments, the recombinant fusion protein or pharmaceutical composition increases proliferation of NK cells.

### Diseases and Disorders

The disclosure provides methods of treating a disease or disorder in a subject comprising administering to the subject a recombinant fusion protein comprising a CTLA-4 antigen binding domain, IL-15Ra sushi domain and IL-15 domain, or a pharmaceutical composition comprising same.

In some embodiments, the disease or disorder is cancer. In some embodiments, the cancer comprises a liquid or a solid tumor.

In some embodiments, the liquid tumor comprises leukemia, acute myeloid leukemia, myeloma, acute myelogenous leukemia (AML), acute lymphoblastic leukemia (ALL), lymphoma, Hodgkin's lymphoma, non-Hodgkin's lymphoma, beta-cell lymphoma, chronic lymphocytic leukemia, or chronic myelogenous leukemia, mantle cell lymphoma, follicular lymphoma, T-cell lymphoma, NK-cell lymphoma, B-cell lymphoma or NKT-cell lymphoma.

In some embodiments, the cancer comprises a solid tumor. In some embodiments, the cancer is selected from the group consisting of melanoma, renal cell carcinoma, mesothelioma, small cell lung cancer, uveal melanoma, bladder cancer, gastric cancer, squamous cell carcinoma of the head and neck, cutaneous carcinoma, non-small cell lung cancer, colorectal cancer, prostate cancer, ovarian cancer, cervical cancer, endometrial carcinoma, breast cancer, pancreatic cancer, urothelial cancer, esophageal cancer, hepatocellular carcinoma, glioblastoma, glioma, or sarcoma.

In some embodiments, the cancer is selected from the group consisting of melanoma, and renal cell carcinoma.

In some embodiments, the cancer is selected from the group consisting of adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin's lymphoma, mantle cell lymphoma, follicular lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstroem macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, epithelioid sarcoma, synovial sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A cancer treated with the recombinant fusion proteins or pharmaceutical compositions comprising same of the disclosure can be staged according to an American Joint Committee on Cancer (AJCC) classification as Stage I, Stage HA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c. Alternatively, or in addition, a cancer can be staged according to the TNM staging system, which divides most types of cancers into 4 stages. Stage 1 usually means that a cancer is relatively small and contained within the organ of origin. Stage 2 cancers have usually not started to spread into surround tissues, but that the tumor is larger than stage 1. In some embodiments, stage 2 means that the cancer has spread into the lymph nodes close to the tumor. Stage 3 cancers are usually larger, and have started to spread into surrounding tissues and lymph nodes. Stage 4, or metastatic cancers, are typically cancers that have spread from the point of origin to other organ(s) in the body.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a recombinant fusion protein or other composition of matter is in direct contact with a cell, or is close enough to induce a desired biological effect in a cell.

As used herein, “monotherapy” refers to the administration of a single active or therapeutic compound to a subject in need thereof. Preferably, monotherapy will involve administration of a therapeutically effective amount of an active compound.

Monotherapy may be contrasted with combination therapy, in which a combination of multiple active compounds is administered, preferably with each component of the combination present in a therapeutically effective amount.

As used herein, “treating” or “treat” describes the management and care of a subject for the purpose of combating a disease, condition, or disorder and includes the administration of a recombinant fusion protein or pharmaceutical composition comprising same of the disclosure to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of recombinant fusion proteins or pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

As used herein, the term “aggressive” indicates a cancer that can grow, form or spread quickly. Cancers termed aggressive may be susceptible to treatment, or they may resist treatment. An aggressive cancer can comprise any sort of cancer. Alternatively, or in addition, the term “aggressive” may describe a cancer that requires a more severe or intense than the usual form of treatment for that cancer.

As used herein, the term “refractory” describes a cancer that does not respond to an attempted form of treatment. Refractory cancers can also be termed resistant cancers.

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 500%6 or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population that is not receiving the recombinant fusion protein, or pharmaceutical composition comprising same, of the disclosure. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population that is not receiving the recombinant fusion protein, or pharmaceutical composition comprising same, of the disclosure. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a recombinant fusion protein or pharmaceutical composition of the disclosure. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with the recombinant fusion protein.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%/6; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Nal Acad Sci USA. 100(5): 2674-8, 2003. In an aspect, cell death occurs by apoptosis.

### Combination Therapies

In some embodiments, it may be desired to administer additional cancer treatments in conjunction with the recombinant fusion proteins or pharmaceutical compositions comprising same. For example, in some treatment regimes, chemotherapeutic agents, antibiotics, additional formulations comprising the recombinant fusion protein of the invention and one or more standard of care agents, etc. are all optionally included with the compositions of the invention. In some embodiments, the recombinant fusion protein is administered in combination with one or more of chemotherapy, a small molecule inhibitor, protein-based or biologic therapy, radiation, surgery, immunotherapy or adoptive cell therapy.

As used herein, the terms “combination treatment,” “combination therapy,” and “co-therapy” are used interchangeably and generally refer to treatment modalities featuring an recombinant fusion protein or pharmaceutical composition comprising the same as provided herein and an additional therapeutic agent or method. Typically, combination treatment modalities are part of a specific treatment regimen intended to provide a beneficial effect from the concurrent action of the therapeutic agent combination. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). In some embodiments, combination treatment comprises administration of two or more therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, separate dosage forms for the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. The therapeutic agents can be administered according to the same or to a different administration interval. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

In some embodiments, combination therapy also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, the additional therapeutic agent is a chemotherapeutic agent (also referred to as an anti-neoplastic agent or anti-proliferative agent), e.g., an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, an immune checkpoint inhibitor, a platinum based antineoplastic agent, a CDK inhibitor, a PARP inhibitor or any anti-neoplastic or anti-proliferative agent known to those of skill in the art.

Exemplary alkylating agents suitable for use according to the combination treatment modalities provided herein include, but are not limited to, cyclophosphamide (Cytoxan; Neosar), chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary suitable anthracyclines include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine-131 tositumomab (Bexxar); alemtuzumab (Campath); ibritumomab (Zevalin; In-111; Y-90 Zevalin); gemtuzumab (Mylotarg); eculizumab (Soliris) or denosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (EMD 72000) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Txempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafemb (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/fasudil hydrochloride; flavopiridol; seliciclib (CYC202; Roscovitine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin), sorafenib (Nexavar), sunitinib (Sutent), ranibizumab, pegaptanib, or vandetanib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary immune checkpoint inhibitors include programmed cell death 1 (PD-1), and CD274 molecule (PD-L1) inhibitors. Exemplary PD-1 inhibitors include pembrolizumab, nivolumab and cemiplimab. Further examples of PD-1 inhibitors include retifanlimab, spartalizumab, camrelizumab, tislelizumab, toripalimab and dostarlimab.

Exemplary PD-L1 inhibitors include atezolizumab, avelumab and durvalumab. Further examples of PD-LI inhibitors include enfavolimab.

Exemplary platinum based antineoplastic agents include Cisplatin and Carboplatin.

Exemplary cyclin dependent kinase (CDK) inhibitors include abemaciclib, palbociclib, and ribociclib.

Exemplary poly (ADP-ribose) polymerase (PARP) inhibitors include talazoparib, olaparib, rucaparib, niraparib and veliparib.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid): temsirolimus (Torisel); arsenic trioxide (Trisenox); verteporfin (Visudyne); mimosine (Leucenol); (1M tegafur−0.4 M 5-chloro-2,4-dihydroxypyrimidine-1 M potassium oxonate) or lovastatin.

Small molecule inhibitors refer to drugs that, because of their small, can be used to target both extracellular and intracellular proteins expressed by cancer cells. Small molecule inhibitors target serine/threonine/tyrosine kinases, matrix metalloproteinases (MMPs), heat shock proteins (HSPs), proteosome and other proteins playing a role in signal transduction pathways. Exemplary small molecule inhibitors include Acitinib, Erlotinib, Imatinib, Gefitinib, Sunitinib, Lapatinib, Nolitinib, Cabozantinib, Crizotinib, Sorafenib, Vemurafenib, Trametinib, Everolimus, Temisorolimus, Ruxolitinib, Bortezomib, Pazopanib, Ruzolitinib, Vandetenib, Bosutinib, Cabozantinib, Ponatinib, Regorafenib, Ibrutinib, Trametinib, Perifosine, Batimistat, Neovastat, Prinomastat, Rebimastat, Ganetespib, Marimastat, Obatoclax, Navitoclax and Carfilzomib.

A protein or biologic based therapy refers to refers to a therapy that includes administration of a therapeutic protein, cell, vector or vaccine. Exemplary biologic based therapies include, but are not limited to, antibody therapies, and adoptive cell therapies such as chimeric antigen receptor T cell (CAR-T) or T Cell Receptor T cell (TCR-T) therapies. Exemplary antibody therapies include, but are not limited to, immune checkpoint inhibitors such as inhibitors of the PD-1 checkpoint (Nivolumab, Pembrolizumab, Atezolizumab, Avelumab, Durvalumab, Cemiplimab), antibodies to growth factors such as EGFR (Cetuximab, Panitumab, Nimotuzumab, Necitumumab) or HER2 (Trastuzumab, Pertuzumab), and antibodies to cancer antigens (e.g., Rituximab, Brentuximab, Gemtuzumab, Ibritumomab, Blinatumumab, Inotuzumab, and others). Exemplary vectors include, but are not limited to, vectors such as adeno-associated (AAV) and lentiviral vectors, which can be used to deliver a nucleic acid encoding a therapeutic protein. Exemplary vaccines include cancer vaccines.

In some embodiments, combination treatment modalities are provided in which the additional therapeutic agent is a cytokine, e.g., G-CSF (granulocyte colony stimulating factor).

In some embodiments, a pharmaceutical composition provided herein may be administered in combination with radiation therapy. Radiation therapy can also be administered in combination with a pharmaceutical composition provided herein and another chemotherapeutic agent described herein as part of a multi-agent therapy. In yet another aspect, a pharmaceutical composition provided herein may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In some preferred embodiments, a pharmaceutical composition provided herein may be administered with an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevec (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets FIt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

In some embodiments, the recombinant fusion protein or pharmaceutical composition comprising same is administered in combination with an adoptive cell therapy. In some embodiments, the adoptive cell therapy is a chimeric antigen receptor T cell (CAR T), TCR T cell therapy or a CAR NK cell therapy.

As used herein, the term “chimeric antigen receptor (CAR)” refers to an artificial transmembrane protein receptor comprising (i) an extracellular domain capable of binding to at least one predetermined CAR ligand or antigen (ii) an intracellular segment comprising one or more cytoplasmic domains derived from signal transducing proteins different from the polypeptide from which the extracellular domain is derived, and (iii) a transmembrane domain. Many different CARs are known in the art, all of which are envisaged as within the scope if the instant invention.

As used herein, TCR T cell therapy refers to T cells engineered to express a TCR capable of binding to at least one predetermined TCR ligand or antigen. Many different TCRs are known in the art, all of which are envisaged as within the scope if the instant invention.

In some embodiments, the recombinant fusion protein comprising a CTLA-4 antigen binding domain, IL-15Ra sushi domain and IL-15 domain, or a pharmaceutical composition comprising same, is administered to a subject daily, every 2 days, every 3 days, every 4 days, every 5 days, every 6 days, every 7 days, every 8 days, every 9 days, every 10 days, every 11 days, every 12 days, every 13 days, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 2 months, every 3 months or every 4 months.

In some embodiments, the recombinant fusion protein comprising a CTLA-4 antigen binding domain, IL-15Ra sushi domain and IL-15 domain, or a pharmaceutical composition comprising same, is administered to a subject daily, every 2 days, every 3 days, every 4 days, every 5 days, every 6 days, every 7 days, every 8 days, every 9 days, every 10 days, every two weeks, every three weeks or monthly.

In some embodiments, the recombinant fusion protein comprising a CTLA-4 antigen binding domain, IL-15Ra sushi domain and IL-15 domain, or pharmaceutical composition comprising same, is administered to a subject once a day. In some embodiments, the recombinant fusion protein or composition comprising same is administered to a subject every two days. In some embodiments, the recombinant fusion protein or composition comprising same is administered to a subject every three days. In some embodiments, the recombinant fusion protein or composition comprising same is administered to a subject every four days. In some embodiments, the recombinant fusion protein or composition comprising same is administered to a subject every five days. In some embodiments, the recombinant fusion protein or composition comprising same is administered to a subject every six days. In some embodiments, the recombinant fusion protein or composition comprising same is administered to a subject every seven days. In some embodiments the recombinant fusion protein or composition comprising same is administered to a subject every eight days. In some embodiments, the recombinant fusion protein or composition comprising same is administered to a subject every nine days. In some embodiments, the recombinant fusion protein or composition comprising same is administered to a subject every ten days. In some embodiments, the recombinant fusion protein or composition comprising same is administered to a subject every eleven days. In some embodiments, the recombinant fusion protein or composition comprising same is administered to a subject every twelve days. In some embodiments, the recombinant fusion protein or composition comprising same is administered to a subject every thirteen days. In some embodiments, the recombinant fusion protein or composition comprising same is administered to a subject every two weeks. In some embodiments, the recombinant fusion protein or composition comprising same is administered to a subject every three weeks. In some embodiments, the recombinant fusion protein or composition comprising same is administered to a subject every month. In some embodiments, the recombinant fusion protein or pharmaceutical composition comprising same is administered to a subject two or more times a year. In some embodiments, the recombinant fusion protein or pharmaceutical composition comprising same is administered to a subject two or more times every two years. In some embodiments, the recombinant fusion protein or pharmaceutical composition comprising same is administered to a subject two or more times every two or more years.

In some embodiments, the recombinant fusion protein or pharmaceutical composition comprising same is administered to a subject once every 7-14 days. In some embodiments, the recombinant fusion protein or pharmaceutical composition comprising the same is administered to a subject once every 10-20 days. In some embodiments, the recombinant fusion protein or pharmaceutical composition comprising the same is administered to a subject once every 5-15 days. In some embodiments, the recombinant fusion protein or pharmaceutical composition comprising the same is administered to a subject once every 15-30 days.

In some embodiments, the recombinant fusion protein or pharmaceutical composition comprising same is administered at least once every 36 hours. In some embodiments, the recombinant fusion protein or pharmaceutical composition comprising same is administered at least once every 48 hours. In some embodiments, the recombinant fusion protein or pharmaceutical composition comprising same is administered at least once every 60 hours. In some embodiments, the recombinant fusion protein or pharmaceutical composition comprising same is administered at least once every 72 hours. In some embodiments, the recombinant fusion protein or pharmaceutical composition comprising same is administered at least once every 84 hours. In some embodiments, the recombinant fusion protein or pharmaceutical composition comprising same is administered at least once every 96 hours. In some embodiments, the recombinant fusion protein or pharmaceutical composition comprising same is administered at least once every 5 days. In some embodiments, the recombinant fusion protein or pharmaceutical composition comprising same is administered at least once every 6 days. In some embodiments, the recombinant fusion protein or pharmaceutical composition comprising same is administered at least once every 7 days. In some embodiments, the recombinant fusion protein or pharmaceutical composition comprising same is administered at least once every 8-10 days. In some embodiments, the recombinant fusion protein or pharmaceutical composition comprising same is administered at least once every 10-12 days. In some embodiments, the recombinant fusion protein or pharmaceutical composition comprising same is administered at least once every 12-15 days. I In some embodiments, the recombinant fusion protein or pharmaceutical composition comprising same is administered at least once every 15-25 days. In some embodiments, the recombinant fusion protein or pharmaceutical composition comprising same is administered at least once every 20-30 days.

In some embodiments, the recombinant fusion protein or pharmaceutical composition comprising same is administered to a subject at least once every 1 month, at least once every 2 months, at least once every 3 months, at least once every 4 months, or at least once every 6 months. In one embodiment, a dose of the recombinant fusion protein or pharmaceutical composition comprising the same is administered at least once every 6-12 months. In some embodiments, the recombinant fusion protein or pharmaceutical composition comprising same is administered quarterly. In some embodiments, the recombinant fusion protein or pharmaceutical composition comprising same is administered daily, weekly, biweekly, monthly or annually. In some embodiments, the recombinant fusion protein or pharmaceutical composition comprising same is administered once, twice, or two or more times a day, a week, a month or a year. In another embodiment, the dose is administered every two, three, four, or at least five years.

In some embodiments, administration of the recombinant fusion protein or pharmaceutical composition comprising same comprises a dosing holiday. For example, the recombinant fusion protein or pharmaceutical composition comprising same is administered to the subject every three days, followed by a week, two weeks, three weeks or a month with no administration, followed by a resumption of dosing. The person of ordinary skill in the art will understand that this dosing holiday is exemplary. Dosing holidays of other duration and frequency are contemplated as within the scope of the instant disclosure.

In some embodiments, the recombinant fusion protein or pharmaceutical composition is administered for at least one week, at least two weeks, at least three weeks, at least 4 weeks, at least 5 weeks, at least 6 weeks, at least 2 months, at least 3 months, at least 4 months, at least 5 months, at least 6 months, at least 7 months, at least 8 months, at least 9 months, at least 10 months, at least 11 months, at least 12 months, at least 14 months, at least 16 months, at least 18 months, at least 20 months, at least 22 months, at least 2 years, at least 2.5 years or at least 3 years.

In some embodiments, the recombinant fusion protein comprising a CTLA-4 antigen binding domain, IL-15Ra sushi domain and IL-15 domain, or a pharmaceutical composition comprising same is administered at a dose of 0.01 μg/kg to 2.0 mg/kg, 0.01 μg/kg to 1.5 mg/kg, 0.01 μg/kg to 1.0 mg/kg, 0.01 μg/kg to 0.9 mg kg, 0.01 μg/kg to 0.8 mg/kg, 0.01 μg/kg to 0.7 mg kg, 0.01 μg/kg to 0.6 mg/kg, 0.01 μg/kg to 0.5 mg/kg, 0.01 μg/kg to 0.4 mg/kg, 0.01 μg/kg to 0.3 mg/kg, 0.01 μg/kg to 0.2 mg/kg, 0.01 μg/kg to 100 μg/kg, 0.01 μg/kg to 50 μg/kg, 0.01 μg/kg to 20 μg/kg, 0.01 μg/kg to 10 μg/kg, 0.1 μg/kg to 2.0 mg/kg, 0.1 μg/kg to 1.5 mg/kg, 0.1 μg/kg to 1.0 mg/kg, 0.1 μg/kg to 0.9 mg kg, 0.1 μg/kg to 0.8 mg/kg, 0.1 μg/kg to 0.7 mg kg, 0.1 μg/kg to 0.6 mg/kg, 0.1 μg/kg to 0.5 mg/kg, 0.1 μg/kg to 0.4 mg/kg, 0.1 μg/kg to 0.3 mg/kg, 0.1 μg/kg to 0.2 mg/kg, 0.1 μg/kg to 100 μg/kg, 0.1 μg/kg to 50 μg/kg, 0.1 μg/kg to 20 μg/kg, 0.1 μg/kg to 10 μg/kg, 1 μg/kg to 2.0 mg/kg, 1 μg/kg to 1.5 mg/kg, 1 μg/kg to 1.0 mg/kg, 1 μg/kg to 0.9 mg kg, 1 μg/kg to 0.8 mg/kg, 1 μg/kg to 0.7 mg kg, 1 μg/kg to 0.6 mg/kg, 1 μg/kg to 0.5 mg/kg, 1 μg/kg to 0.4 mg/kg, 1 μg/kg to 0.3 mg/kg, 1 μg/kg to 0.2 mg/kg, 1 μg/kg to 100 μg/kg, 1 μg/kg to 50 μg/kg, 1 μg/kg to 20 μg/kg, 1 μg/kg to 10 μg/kg, 10 μg/kg to 2.0 mg/kg, 10 μg/kg to 1.5 mg/kg, 10 μg/kg to 1.0 mg/kg, 10 μg/kg to 0.9 mg kg, 10 μg/kg to 0.8 mg/kg, 10 μg/kg to 0.7 mg kg, 10 μg/kg to 0.6 mg/kg, 10 μg/kg to 0.5 mg/kg, 10 μg/kg to 0.4 mg/kg, 10 μg/kg to 0.3 mg/kg, 10 μg/kg to 0.2 mg/kg, 10 μg/kg to 100 μg/kg, 10 μg/kg to 50 μg/kg, 10 μg/kg to 20 μg/kg, 50 μg/kg to 1.0 mg/kg, 50 μg/kg to 0.5 mg/kg, 50 μg/kg to 100 μg/kg, 100 μg/kg to 2.0 mg/kg, 100 μg/kg to 1.5 mg/kg, 100 μg/kg to 1.0 mg/kg, 100 μg/kg to 0.5 mg/kg, 100 μg/kg to 0.3 mg/kg, or 100 μg/kg to 200 μg/kg.

In some embodiments, the recombinant fusion protein comprising a CTLA-4 antigen binding domain, IL-15Ra sushi domain and IL-15 domain, or a pharmaceutical composition comprising same is administered at a dose of 0.01 μg/kg to 2.0 mg/kg. In some embodiments, the recombinant fusion protein or pharmaceutical composition comprising same is administered at a dose of 0.1 μg/kg to 1.0 mg/kg. In some embodiments, the recombinant fusion protein or pharmaceutical composition comprising same is administered at a dose of 1.0 μg/kg to 0.5 mg/kg. In some embodiments, the recombinant fusion protein or pharmaceutical composition comprising same is administered at a dose of 10 μg/kg to 300 μg/kg. In some embodiments, the recombinant fusion protein or pharmaceutical composition comprising same is administered at a dose of 50 μg/kg to 200 μg/kg. In some embodiments, the recombinant fusion protein or pharmaceutical composition comprising same is administered at a dose of 100 μg/kg to 200 μg/kg.

In some embodiments, the recombinant fusion protein or pharmaceutical composition comprising the same is administered to a subject in a dose ranging from 10 μg to 500 mg, 10 μg to 400 mg, 10 μg to 300 mg, 10 μg to 200 mg, 10 μg to 100 mg, 10 μg to 75 mg, 10 μg to 50 mg, 10 μg to 40 mg, 10 μg to 30 mg, 10 μg to 20 mg, 10 μg to 10 mg, 100 μg to 500 mg, 100 μg to 400 mg, 100 μg to 300 mg, 100 μg to 200 mg, 100 μg to 100 mg, 100 μg to 50 mg, 100 μg to 40 mg, 100 μg to 30 mg, 100 μg to 20 mg, 100 μg to 10 mg, 300 μg to 500 mg, 300 μg to 400 mg, 300 μg to 300 mg, 300 μg to 200 mg, 300 μg to 100 mg, 300 μg to 50 mg, 300 μg to 40 mg, 300 μg to 30 mg, 300 μg to 20 mg, 300 μg to 10 mg, 500 μg to 500 mg, 500 μg to 400 mg, 500 μg to 300 mg, 500 μg to 200 mg, 500 μg to 100 mg, 500 μg to 50 mg, 500 μg to 40 mg, 500 μg to 30 mg, 500 μg to 20 mg, 500 μg to 10 mg, 600 μg to 500 mg, 600 μg to 400 mg, 600 μg to 300 mg, 600 μg to 200 mg, 600 μg to 100 mg, 600 μg to 50 mg, 600 μg to 40 mg, 600 μg to 30 mg, 600 pg to 20 mg, 600 μg to 10 mg, 700 μg to 500 mg, 700 μg to 700 mg, 700 μg to 300 mg, 700 μg to 200 mg, 700 μg to 100 mg, 700 μg to 50 mg, 700 μg to 40 mg, 700 μg to 30 mg, 700 μg to 20 mg, 700 μg to 10 mg, 800 μg to 500 mg, 800 μg to 400 mg, 800 μg to 300 mg, 800 μg to 200 mg, 800 μg to 100 mg, 800 μg to 50 mg, 800 μg to 40 mg, 800 μg to 30 mg, 800 μg to 20 mg, 800 μg to 10 mg, 1 mg to 500 mg, 1 mg to 400 mg, 1 mg to 300 mg, 1 mg to 200 mg, 1 mg to 100 mg, 1 mg to 50 mg, 1 mg to 40 mg, 1 mg to 30 mg, 1 mg to 20 mg, 1 mg to 10 mg, 5 mg to 500 mg, 5 mg to 400 mg, 5 mg to 300 mg, 5 mg to 200 mg, 5 mg to 100 mg, 5 mg to 50 mg, 5 mg to 40 mg, 5 mg to 30 mg, 5 mg to 20 mg, 5 mg to 10 mg, 10 mg to 500 mg, 10 mg to 400 mg, 10 mg to 300 mg, 10 mg to 200 mg, 10 mg to 100 mg, 10 mg to 50 mg, 20 mg to 500 mg, 20 mg to 300 mg, 20 mg to 200 mg, 5 mg to 100 mg, or 20 mg to 50 mg.

In some embodiments, the recombinant fusion protein or pharmaceutical composition comprising the same is administered to a subject in a dose ranging from 0.05 μg to 1,000 mg. In some embodiments, the recombinant fusion protein or pharmaceutical composition comprising the same is administered to a subject in a dose ranging from 5 pg to 1,000 mg. In some embodiments, the recombinant fusion protein or pharmaceutical composition comprising the same is administered to a subject in a dose ranging from 50 μg to 500 mg. In some embodiments, the recombinant fusion protein or pharmaceutical composition comprising the same is administered to a subject in a dose ranging from 50 μg to 100 mg. In some embodiments, the recombinant fusion protein or pharmaceutical composition comprising the same is administered to a subject in a dose ranging from 100 μg to 500 mg. In some embodiments, the recombinant fusion protein or pharmaceutical composition comprising the same is administered to a subject in a dose ranging from 100 μg to 100 mg. In some embodiments, the recombinant fusion protein or pharmaceutical composition comprising the same is administered to a subject in a dose ranging from 500 μg to 1000 mg. In some embodiments, the recombinant fusion protein or pharmaceutical composition comprising the same is administered to a subject in a dose ranging from 500 μg to 100 mg. In some embodiments, the recombinant fusion protein or pharmaceutical composition comprising the same is administered to a subject in a dose ranging from 1 mg to 500 mg. In some embodiments, the recombinant fusion protein or pharmaceutical composition comprising the same is administered to a subject in a dose ranging from 1 mg to 100 mg. In some embodiments, the recombinant fusion protein or pharmaceutical composition comprising the same is administered to a subject in a dose ranging from 500 μg to 50 mg. In some embodiments, the recombinant fusion protein or pharmaceutical composition comprising the same is administered to a subject in a dose ranging from 500 μg to 40 mg. In some embodiments, the recombinant fusion protein or pharmaceutical composition comprising the same is administered to a subject in a dose ranging from 500 μg to 36 mg. In some embodiments, the recombinant fusion protein or pharmaceutical composition comprising the same is administered to a subject in a dose ranging from 500 μg to 30 mg. In some embodiments, the recombinant fusion protein or pharmaceutical composition comprising the same is administered to a subject in a dose ranging from 500 μg to 20 mg. In some embodiments, the recombinant fusion protein or pharmaceutical composition comprising the same is administered to a subject in a dose ranging from 700 μg to 100 mg. In some embodiments, the recombinant fusion protein or pharmaceutical composition comprising the same is administered to a subject in a dose ranging from 700 μg to 50 mg. In some embodiments, the recombinant fusion protein or pharmaceutical composition comprising the same is administered to a subject in a dose ranging from 700 μg to 40 mg. In some embodiments, the recombinant fusion protein or pharmaceutical composition comprising the same is administered to a subject in a dose ranging from 700 μg to 36 mg. In some embodiments, the recombinant fusion protein or pharmaceutical composition comprising the same is administered to a subject in a dose ranging from 700 μg to 30 mg. In some embodiments, the recombinant fusion protein or composition comprising the same is administered to a subject in a dose ranging from 700 μg to 20 mg. In some embodiments, the recombinant fusion protein or pharmaceutical composition comprising the same is administered to a subject in a dose ranging from 800 μg to 100 mg. In some embodiments, the recombinant fusion protein or pharmaceutical composition comprising the same is administered to a subject in a dose ranging from 800 μg to 50 mg. In some embodiments, the recombinant fusion protein or pharmaceutical composition comprising the same is administered to a subject in a dose ranging from 5 mg to 1,000 mg. In some embodiments, the recombinant fusion protein or pharmaceutical composition comprising the same is administered to a subject in a dose ranging from 10 mg to 500 mg. In some embodiments, the recombinant fusion protein or pharmaceutical composition comprising the same is administered to a subject in a dose ranging from 10 mg to 100 mg.

In one embodiment, a single one time dose of the recombinant fusion protein or pharmaceutical composition comprising the same is administered to a subject. In another embodiment, a total of two doses are administered to the subject. In another embodiment, a total of two or more doses are administered to the subject. In some embodiments, a single dose is administered in a single injection. In some embodiments, a single dose is administered in multiple injections, e.g. 1, 2, 3, 4, or more injections.

In some embodiments, the recombinant fusion protein or pharmaceutical composition comprising same is administered by intravenous, intra-arterial, subcutaneous, intratumoral or intramuscular injection of a liquid preparation. In some embodiments, liquid formulations include solutions, suspensions, dispersions, emulsions, oils and the like. In some embodiments, the recombinant fusion proteins or pharmaceutical compositions are administered intravenously, and are thus formulated in a form suitable for intravenous administration. In some embodiments, the recombinant fusion proteins or pharmaceutical compositions are administered intra-arterially, and are thus formulated in a form suitable for intra-arterial administration. In some embodiments, the recombinant fusion proteins or pharmaceutical compositions are administered subcutaneously, and are thus formulated in a form suitable for subcutaneous administration. In some embodiments, the recombinant fusion proteins or pharmaceutical compositions are administered intratumorally, and are thus formulated in a form suitable for intratumoral administration.

In some embodiments, compositions for use in the methods disclosed herein comprise solutions or emulsions, which in some embodiments are aqueous solutions or emulsions comprising a safe and effective amount of the compounds disclosed herein and optionally, other compounds, intended for intravenous or subcutaneous administration.

In some embodiments, administration of the recombinant fusion proteins described herein, or pharmaceutical compositions comprising same does not substantially increase a level of interferon gamma (IFNγ) in a peripheral blood sample from the subject. In some embodiments, administration of the recombinants fusion proteins of the disclosure or pharmaceutical compositions comprising increases a level of interferon gamma (IFN-γ) in a peripheral blood sample from the subject less than administration of an equimolar amount of IL-15 or IL-15 in a complex with the IL-15Ra sushi domain. In some embodiments, the peripheral blood sample comprises whole blood. In some embodiments, the peripheral blood sample comprises plasma. In some embodiments, the peripheral blood sample comprises serum.

In some embodiments, administration of the recombinant fusion proteins described herein, or pharmaceutical compositions comprising same increases proliferation of immune cells in a subject. Alternatively, or in addition, administration of the recombinant fusion proteins described herein, or pharmaceutical compositions comprising same increases the number immune cells in a subject. The number of immune cells can be affected by immune cell survival, proliferation, or a combination thereof. Alternatively, or in addition, administration of the recombinant fusion proteins described herein, or pharmaceutical compositions comprising same increases the number of active immune cells in a subject. In some embodiments, the proliferation, survival, activity or number of immune cells is increased while not substantially increasing the level of IFNγ in the subject. In some embodiments, the immune cells comprise natural killer (NK) cells. In some embodiments, the immune cells comprise T cells, for example CD8+ T cells. In some embodiments, the immune cells comprise a combination of NK cells and T cells. Increases in the number of immune cells can persist for at least at least 2, 3, 4, 5, 6, 7 or more days after administration of the recombinant fusion proteins and pharmaceutical compositions described herein.

Methods of assaying levels of cytokines such as IL-2, IL-4, IL-6, IL-8, IL-10, TNFα and IFNγ will be known to persons of ordinary skill in the art, and include, inter alia, immunoassays such as ELISA assays on whole blood or plasma samples drawn from the subject. Tests for IFNγ are known in the art and are commercially available. Exemplary tests include the QuantiFERON-TB Gold (QFT) test. Baseline levels of IFNγ vary according to test, and can be set by a reference sample specific to the test. However, levels that are less than 2 picograms (p)/mL, less than 3 pg/mL, less than 5 pg/mL, or less than 10 pg/mL are generally considered within the normal range for IFNγ for a healthy subject. Accordingly, levels of IFNγ that are less 3 pg/mL, less than 5 pg/mL, less than 10 pg/mL or less than 20 pg/mL after administration of the recombinant fusion proteins and pharmaceutical compositions comprising same are considered to not be substantial increases of IFNγ. Similarly, increases in IFNγ of less than 1.1, 1.2, 1.3, 1.4, 1.5, 1.6, 1.7, 1.8, 1.9 or 2 fold over a baseline level of IFNγ measured before administration of the recombinant fusion proteins and pharmaceutical compositions described herein is not a substantial increase in IFNγ. Tests for IL-6 are similarly known in the art, and are commercially available, for example from QuestDiagnostics.

In some embodiments, the level of IFNγ is measured prior to administration of the recombinant fusion proteins or pharmaceutical compositions comprising same described herein to establish a baseline, and then after administration of the recombinant fusion proteins or pharmaceutical compositions described herein to determine the amount by which the level of IFNγ has increased upon administration. In some embodiments, the level of IFNγ is measured about 1 hour, 2 hours, 2 hours, 3 hours, 5 hours, 6 hours, 8 hours, 10 hours, 12 hours, 14 hours, 16 hours, 18 hours, 20 hours, 24 hours, 36 hours, 48 hours, or 72 hours, or a combination thereof, after administration of recombinant fusion proteins or pharmaceutical compositions described herein.

Alternatively, levels of IFNγ following administration of the fusion proteins described herein can be compared to administration of a comparable IL-15Ra sushi/IL-15 fusion protein lacking the anti-CTLA-4 antigen binding domain. In some embodiments, administration of a fusion protein of the disclosure to a subject increases the level of IFNγ of the subject less than the administration of a comparable IL-15Ra sushi/IL-15 fusion protein lacking the anti-CTLA-4 antigen binding domain. In some embodiments, administration of the fusion protein described herein causes an increase in IFNγ that is less than 60%, less than 50%, less than 40%, less than 30%, less than 20%, less than 10% or less than 5% of the increase seen with a comparable IL-15Ra sushi/IL-15 fusion protein lacking the anti-CTLA-4 antigen binding domain.

In some embodiments, administration of the fusion proteins described herein results in a ratio of IL-6 to IFNγ in the subject that is greater than or equal to 2:1, 3:1, 4:1, 5:1, 6:1, 7:1, 8:1, 9:1, 10:1, 15:1, 20: 1, 25:1, 30:1, 35:1, 40:1, 45:1 or 50:1. In some embodiments, administration of the fusion proteins described herein results in a ratio of IL-6 to IFNγ in the subject that is greater than or equal to 2:1, 3:1, 4:1, 5:1, 6:1, 7:1, 8:1, 9:1, or 10:1. In some embodiments, administration of the fusion proteins described herein results in a ratio of IL-6 to IFNγ in the subject that is greater than or equal to 3:1. In some embodiments, administration of the fusion proteins described herein results in a ratio of IL-6 to IFNγ in the subject that is greater than or equal to 5:1. In some embodiments, administration of the fusion proteins described herein results in a ratio of IL-6 to IFNγ in the subject that is greater than or equal to 8:1. In some embodiments, administration of the fusion proteins described herein results in a ratio of IL-6 to IFNγ in the subject that is greater than or equal to 10:1. In some embodiments, administration of the fusion proteins described herein results in a ratio of IL-6 to IFNγ in the subject that is greater than or equal to 15:1. In some embodiments, administration of the fusion proteins described herein results in a ratio of IL-6 to IFNγ in the subject that is greater than or equal to 20:1. In some embodiments, administration of the fusion proteins described herein results in a ratio of IL-6 to IFNγ that is between 2:1 and 50:1, between 3:1 and 30:1, between 5:1 and 20:1, between 3:1 and 10:1, between 3:1 and 5:1, between 5:1 and 30:1, between 5:1 and 20:1, between 5:1 and 10:1, between 10:1 and 50:1, or between 10:1 and 20:1. In some embodiments, administration of the fusion proteins described herein results in a ratio of IL-6 to IFNγ that is between 3:1 and 30:1. In some embodiments, administration of the fusion proteins described herein results in a ratio of IL-6 to IFNγ that is between 3:1 and 20:1. In some embodiments, administration of the fusion proteins described herein results in a ratio of IL-6 to IFNγ that is between 2:1 and 20:1.

### Kits and Articles of Manufacture

The present invention further provides a kit comprising the recombinant fusion protein of the disclosure or pharmaceutical composition comprising same for preventing, treating or delaying cancer in a subject, wherein the kit comprises one or more doses of pharmaceutical composition and instructions on how to use the pharmaceutical preparation or composition.

In some embodiments, the kit comprises syringes, vials labels, and/or instructions on how to use the pharmaceutical preparation or composition.

In some embodiments of the kits of the disclosure, the various constituents of the compositions come pre-measured and/or prepackaged and/or ready for use without additional measurement, etc. The present invention also optionally comprises kits for conducting/using the methods and/or the compositions of the invention. In particular, these kits optionally include, e.g., appropriate recombinant fusion protein (and optionally additional reagents for performing combination treatments as described supra). Additionally, such kits can also comprise appropriate excipients (e.g., pharmaceutically acceptable excipients) for performing therapeutic and/or prophylactic treatments of the invention. Such kits optionally contain additional components for the assembly and/or use of the compositions of the invention including, but not limited to, e.g., diluents, etc.

The compositions described herein are optionally packaged to include all (or almost all) necessary components for performing the methods of the invention or for using the compositions of the invention (optionally including, e.g., written instructions for the use of the methods/compositions of the invention). For example, the kits can optionally include such components as, e.g., buffers, reagents, serum proteins, antibodies, substrates, etc. In the case of prepackaged reagents, the kits optionally include pre-measured or pre-dosed amounts that are ready to incorporate into the methods without measurement, e.g., pre-measured fluid aliquots, or pre-weighed or pre-measured solid reagents that can be easily reconstituted by the end-user of the kit.

Such kits also typically include appropriate instructions for performing the methods of the invention and/or using the compositions of the invention. In some embodiments, the components of the kits/packages are provided in a stabilized form, so as to prevent degradation or other loss during prolonged storage, e.g., from leakage. A number of stabilizing processes/agents are widely used for reagents, etc. that are to be stored, such as the inclusion of chemical stabilizers (i.e., enzymatic inhibitors, microbicides/bacteriostats, anticoagulants), and the like.

The following examples are presented in order to more fully illustrate the preferred embodiments of the invention. They should in no way be construed, however, as limiting the broad scope of the invention.

### Examples

### Example 1: Construction of an Anti-CTLA-4-IL-15Ra-Sushi-IL-15 Fusion Protein and Expression Vector

A schematic of the CTLA-4 antibody fused to the IL-15Ra sushi domain and IL-15, as well as a CTLA-4 antibody IL-15 fusion protein used in this example, are shown in FIG. 1A.

DNA sequences encoding the following constructs were synthesized by GENEWIZ:
(1) an anti-CTLA-4 antibody heavy chain, derived from Ipilimumab, with a constant region modified to promote heterodimerization (the “hole” construct, of a “knob into hole” heterodimerization pair) fused to an IL-15Ra sushi domain, each separated by a G4S linker, SEQ ID NO: 18 corresponding to amino acid sequence SEQ ID NO: 16.
(2) an anti-CTLA-4 antibody heavy chain, derived from Ipilimumab, with a constant region modified to promote heterodimerization (the “knob” construct), SEQ ID NO: SEQ ID NO: 19, corresponding to amino acid sequence SEQ ID NO: 10; and
(3) an anti-CTLA-4 antibody light chain (Ipilimumab), SEQ ID NO: 17, corresponding to amino acid sequence SEQ ID NO: 9.

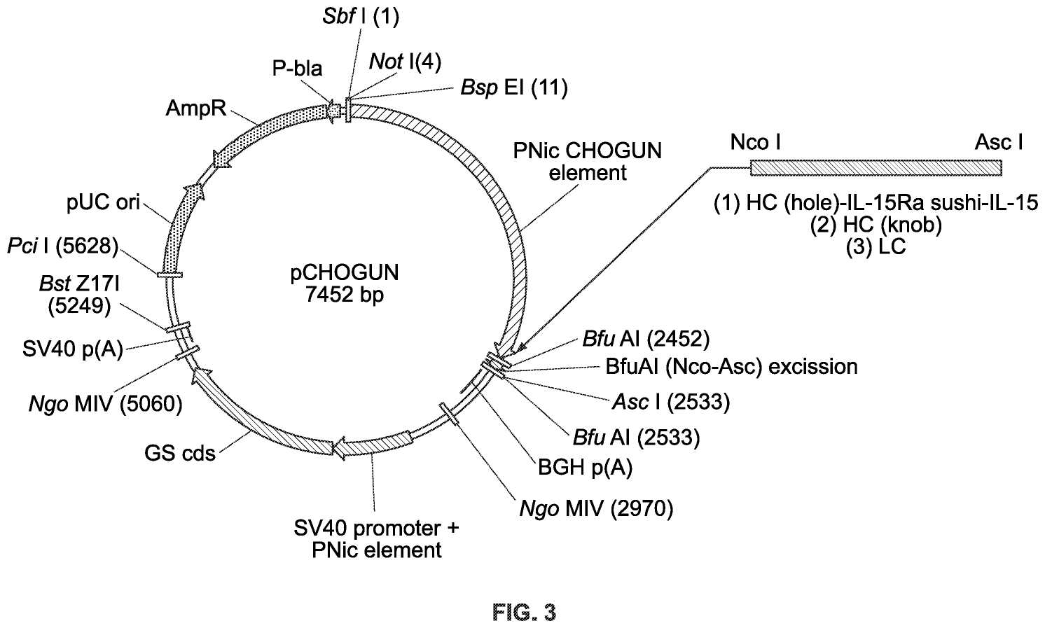

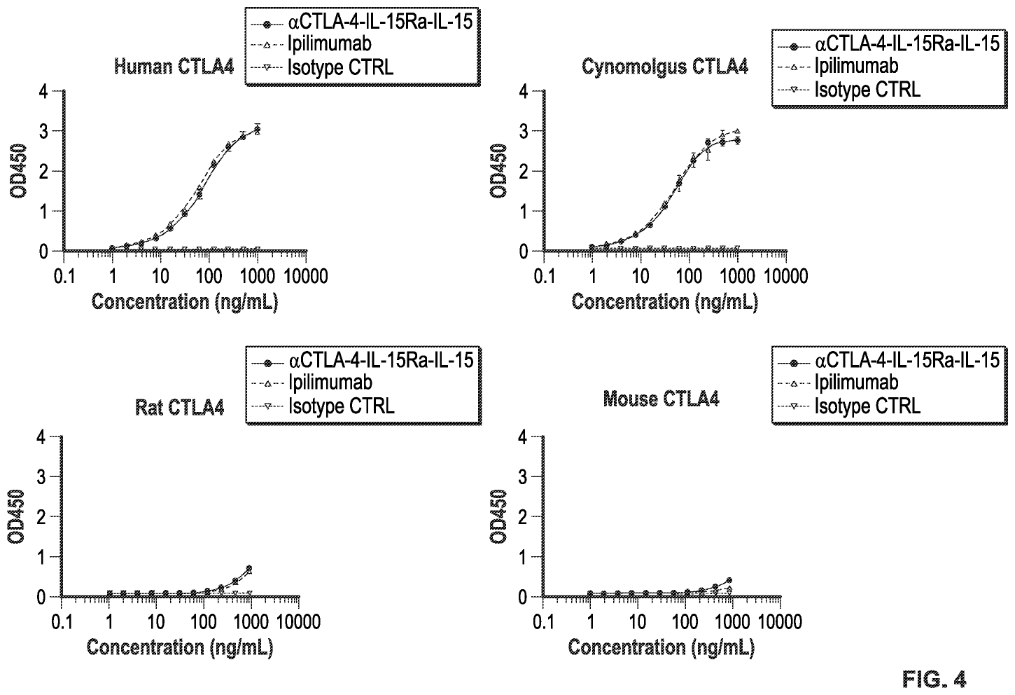

Expression vector pCHOGUN was obtained from Horizon Discovery (Cambridge, UK) under a licensing agreement. Construction of the anti-CTLA-4-IL-15Ra-sushi-IL-15 fusion protein expression plasmids was carried out as outlined in FIG. 3. Briefly, pCHOGUN vector was linearized by restriction enzyme BfuAI, and gene insert fragments of the two heavy chains and the light chain were purified following double restriction enzyme digestion by NcoI and AscI. The linearized vector pCHOGUN/BfuAI and the purified gene insert fragments of the two heavy chains and the light chain were each ligated per standard protocol, and then transformed into E. coli DH5α competent cells. The transformed DH5a cells were plated and incubated overnight at 37° C. Plasmids with the heavy or light chain sequence insert were isolated and confirmed by restriction enzyme digestion. The plasmid constructs containing all three constructs were identified, and inserts were additionally confirmed by DNA sequencing. The three plasmid sequenced verified plasmids were used to transfect the host cell line HD-BIOP3 to generate the production cell line for anti-CTLA-4-IL-15Ra-sushi-IL-15 fusion protein expression.

Expression vectors for heavy chains for an Ipilimumab-IL-15 fusion protein (no IL-15Ra sushi domain, see FIG. 1A, middle diagram) were cloned using a similar strategy to that described above. This construct used the same light chain construct as above. The sequences for the Anti-CTLA-4-IL-15 fusion protein are presented in Table 3 below.

TABLE 3
Anti-CTLA-4-IL-15 Fusion Protein

| Name | Sequence |
|---|---|
| HC1-CTLA- | SEQ ID NO: 38 |
| 4-IL-15; DNA | |
| sequence | |
| HC1 -CTLA- | QVQLVESGGGVVQPGRSLRLSCAASGFTFSSYTMHWVRQAPGKGLEWVTFISYDG |
| 4-IL-15 | NNKYYADSVKGRFTISRDNSKNTLYLQMNSLRAEDTAIYYCARTGWLGPFDYWGQ |
| | GTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKRVEPKS |
| | CDKTHTCPPCPAPEFLGGPAVFLFPPKPKDTLMISRTPEVTCVVVDVSHEDPEVK |
| | FNWYVDGVEVHNAKTKPREEQYNSTYRVVSVLTVLHQDWINGKEYKCKVSNKALP |
| | APIEKTISKAKGQPREPQVYTLPPCREEMTKNQVSLWCLVKGFYPSDIAVEWESN |
| | GQPENNYKTTPPVLDSDGSFFLYSKLTVDKSRWQQGNVFSCSVMHEALHAHYTQK |
| | SLSLSPGKGGGGSGGGGSNWVNVISDLKKIEDLIQSMHIDATLYTESDVHPSCKV |
| | TAMKCFLLELQVISLESGDASIHDTVENLIILANNSLSSNGNVTESGCKECEELE |
| | EKNIKEFLQSFVHIVQMFINTS (SEQ ID NO: 39) |
| HC2-CTLA-4- | SEQ ID NO: 40 |
| IL-15; DNA | |
| sequence | |
| HC2-CTLA-4- | QVQLVESGGGVVQPGRSLRLSCAASGFTFSSYTMHWVRQAPGKGLEWVTFISYDG |
| IL-15 | NNKYYADSVKGRFTISRDNSKNTLYLQMNSLRAEDTAIYYCARTGWLGPFDYWGQ |
| | GTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKRVEPKS |
| | CDKTHTCPPCPAPEFLGGPAVFLFPPKPKDTIMISRTPEVTCVVVDVSHEDPEVK |
| | FNWYVDGVEVHNAKTKPREEQYNSTYRVVSVLTVLHQDWLNGKEYKCKVSNKALP |
| | APIEKTISKAKGQPREPQVCTLPPSREEMTKNQVSLSCAVKGFYPSDIAVEWESN |
| | GQPENNYKTTPPVLDSDGSFFLVSKLTVDKSRWQQGNVFSCSVMHEALHAHYTOK |
| | SLSLSPGK (SEQ ID NO: 41) |

TABLE 4
BS3 anti-CTLA-4-IL15-IL15RA Sushi Heavy chain sequences

| Name | Protein Sequence | DNA Sequence |
|---|---|---|
| BS3 HC | SEQ ID NO: 10 | SEQ ID NO: 19 |
| (knob) | | |
| BS3 HC | SEQ ID NO: 42 | SEQ ID NO: 43 |
| (hole) | | |

The BS3 construct uses a light chain of SEQ ID NO: 9. T366W in FIG. 1E refers to position 367 of SEQ ID NO: 10, while Y407T refers to position 408 of SEQ ID NO: 42.

To evaluate temperature stability, samples were first diluted to 1 mg/ml in 1×PBS, pH 7.4 and 10 μl of sample per capillary was loaded in triplicate using the standard protocol for the Prometheus NT.48 Instrument (NanoTemper Technologies Inc.) Samples were subjected to a temperature ramp of 1.0° C./min from 20° C. to 95° C. and fluorescence intensity at 350 nm and 330 nm was continuously monitored. Data was analyzed with the Prometheus NT ThermControl software (NanoTemper Technologies Inc.).

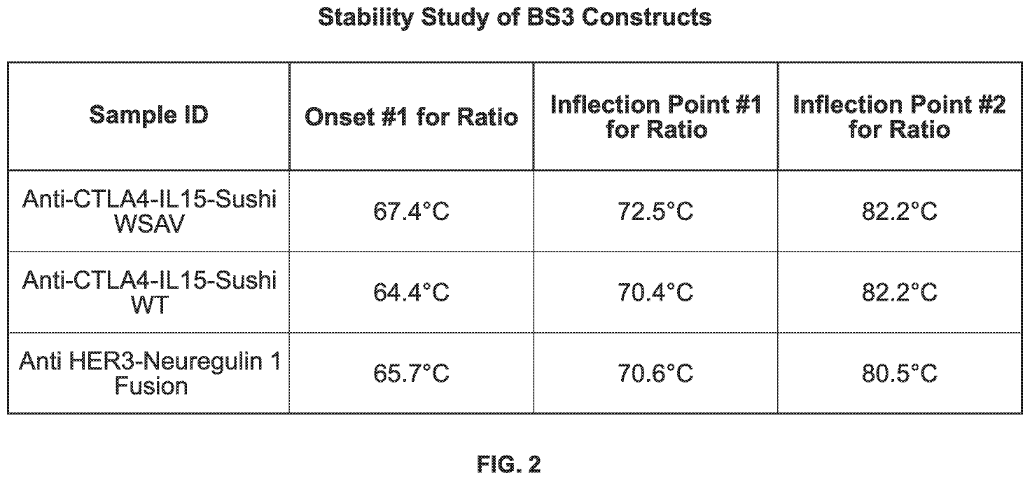

The results are shown in FIG. 2. Results from an anti-HER3-neuregulin-1 fusion protein are provided for reference. In FIG. 2, Anti-CTLA4-IL15-Sushi WT refers to the BS3 construct shown in FIG. 1E, right panel, with the Y407T hole and T366W knob mutations in the constant regions (corresponding to positions 408 and 367 of SEQ ID NOS: 42 and 10 respectively). Anti-CTLA4-IL15-Sushi WSAV refers to the construct comprising heavy chains of SEQ ID NOS: 10 and 16, with a knob heavy chain carrying a S366W substitution, and the hole heavy chain carrying T366S, L368A and Y407V substitutions (note that these correspond to positions 367, 369 and 408, respectively in SEQ ID NOS: 10 and 16). The WSAV variant was used for additional experiments, as the knob-hole construct it was more stable.

### Example 2: Anti-CTLA-4-IL-15Ra-Sushi-IL-15 Fusion Proteins Interact with CTLA-4

CTLA-4 antibody, IL-15Ra sushi domain and IL-15 fusion constructs (anti-CTLA-4-IL-15Ra-sushi-IL-15) interact with CTLA-4 and neutralize the inhibitory activity of CTLA-4 to restore the CD28 costimulatory signaling pathway.

The binding activity of the anti-CTLA-4-IL-15Ra-sushi-IL-15 fusion protein to recombinant CTLA-4 protein which originated from different species, including human, cynomolgus monkey, rat, and mouse, was determined by Enzyme Linked Immunosorbent Assay (ELISA). Briefly, 96-well plates were coated overnight at 4° C. with 1 pg/mL of CTLA-4 protein diluted in PBS. Plates were blocked with 1% bovine serum albumin (BSA) for an hour at room temperature, and then incubated for additional 2 hours with the anti-CTLA-4-IL-15Ra-sushi-IL-15 fusion protein, Ipilimumab, or human IgG1 isotype control, at a 2-fold serial dilution starting from 1 μg/mL. Goat anti-human IgG (Fc specific)-peroxidase antibody (Sigma, 1:5000 dilution) was added to the plates and incubated for 1 hour at room temperature. After washing, the 3, 3′, 5, 5′-Tetramethylbenzidine (TMB) substrate was added to the wells to develop color and the absorbance at 450 nm was measured using the Multiskan plate reader (Thermo). Optical Density 450 (OD450) values were plotted against antibody concentration. As shown in FIG. 4, similar to Ipilimumab, the anti-CTLA-4-IL-15Ra-sushi-IL-15 fusion protein can bind CTLA-4 protein from humans and cynomolgus monkeys, but not rats or mice.

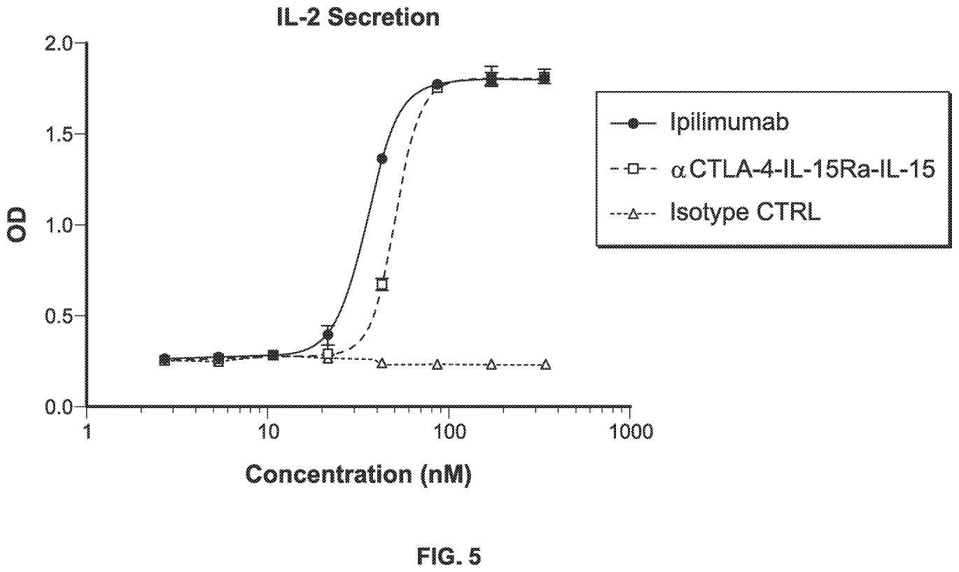

The CTLA-4 blockade activity of the anti-CTLA-4-IL-15Ra-sushi-IL-15 fusion protein was verified in a cell-based anti-CTLA-4 bioactivity assay which was developed by Genscript. Briefly, human CD80-expressing cell line GS-C1 was seeded in 96-well cell culture plates at 50 μL/well and a density of 1×10⁶ cells/mL. The anti-CTLA-4-IL-15Ra-sushi-IL-15 fusion protein, Ipilimumab and human IgG1 isotype control were prepared at a 2-fold serial dilution starting from 2.07 μM (3× final concentration) and then added to the plates at 50 μL/well. GS-J1, a CD28-expressing T cell line, was suspended to a density of 2×10⁶ cells/mL, mixed with human CTLA4-Fc fusion protein (Sino biological, 6 mg/mL) and PHA (Sigma, 15 mg/mL), and then added to the plates at 50 μL/well to initiate the assay. After 24 hours of incubation at 37° C. and 5% CO₂, conditioned media in each well was harvested and IL-2 secretion was tested using a human IL-2 Quantikine ELISA kit per manufacturer's instructions (R&D). As shown in FIG. 5, both the anti-CTLA-4-IL-15Ra-sushi-IL-15 fusion protein and Ipilimumab blocked the CTLA-4/CD80 interaction and restored the CD28 costimulatory signaling pathway, which led to IL-2 secretion.

### Example 3: Anti-CTLA-4-IL-15Ra-Sushi-IL-15 Fusion Protein Retains Antibody-Dependent Cellular Cytotoxicity Activity Against CTLA-4 Expressing Target Cells

Antibody-dependent cell-mediated cytotoxicity (ADCC) activity assessment was performed using the ADCC Reporter Bioassay developed by Promega. Briefly, engineered Jurkat cells stably expressing the FcγRIIIa receptor and an NFAT (nuclear factor of activated T-cells) response element driving expression of firefly luciferase were used as effector cells. When the antibody binds to antigens on the target cell surface and to FcγRIIIa receptors on effector cell surface, multiple cross-linking of the two cell types occurs, leading to pathway activation of ADCC MOA (mechanism of action) that can be quantified through the luciferase produced by NFAT pathway activation. The Jurkat-NFAT cell line was obtained from Promega, and Raji-hCTLA4 cell line (Target cells) was obtained from InvivoGen. Raji-hCTLA4 cells were seeded in 96-well white-wall cell culture plates at 25 μl/well and a density of 6×10⁵ cells/mL. Anti-CTLA-4-IL-15Ra-sushi-IL-15 fusion protein (SEQ ID NOS: 9, 10 and 16) and ipilimumab, prepared in 5-fold serial dilutions (starting from 1800 nM), were then added to the plates at 25 L/well. Jurkat-NFAT cells were finally seeded at 25 μl/well and a density of 3×10⁶ cells/mL. After 20 hours of incubation at 37° C. and 5% CO₂, activity of the anti-CTLA-4-IL-15Ra-sushi-IL-15 fusion protein in promoting NFAT pathway activation was assessed using the Bio-Glo luminescent kit (Promega). Luminescence readout in each well was plotted against the antibody concentration.

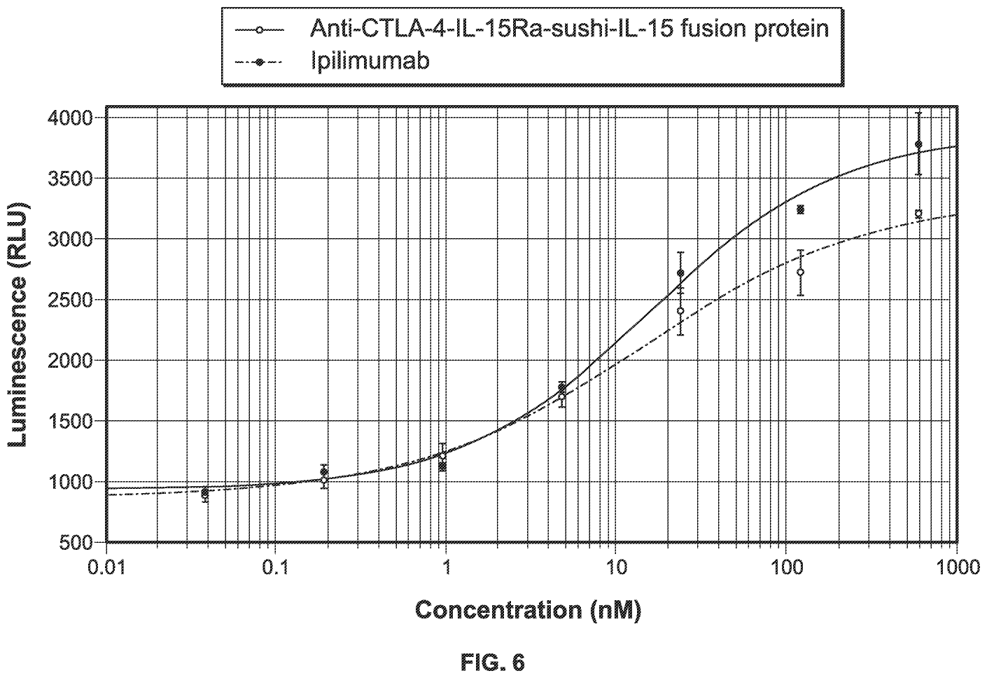

As shown in FIG. 6 both the anti-CTLA-4-IL-15Ra-sushi-1L-15 fusion protein and Ipilimumab can induce luciferase production to a similar level.

### Example 4: Anti-CTLA-4-IL-15Ra-sushi-IL-15 Fusion Proteins Interact with theft Subunit of IL-2 Receptor (IL2Rβ)

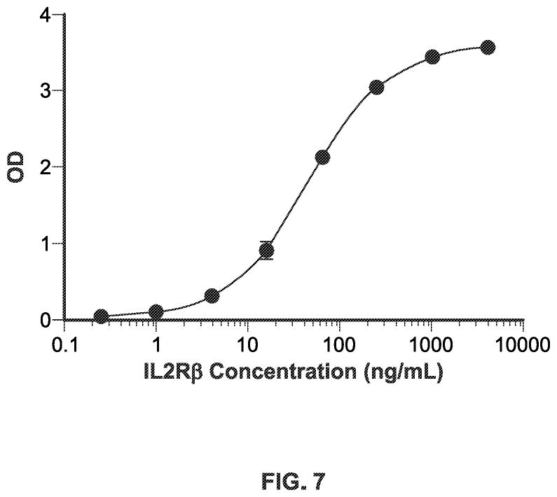

The binding activity of the anti-CTLA-4-IL-15Ra-sushi-IL-15 fusion protein (SEQ ID NOS: 9, 10 and 16) to IL2RO was determined by Enzyme Linked Immunosorbent Assay (ELISA). Briefly, 96-well plates were coated with 2 sg/mL of an anti-idiotypic antibody against the anti-CTLA-4-IL-15Ra-sushi-IL-15 fusion protein (clone 31E2E3, generated in house) diluted in PBS overnight at 4° C. Plates were blocked with 1% BSA for 1 hour at room temperature, and then sequentially incubated with 1 pg/mL of anti-CTLA-4-IL-15Ra-sushi-IL-15 fusion protein and 4-fold serially diluted human IL2Rβ/his-tagged protein (starting from 4 pg/mL). His-tagged recombinant human IL2Rβ protein was obtained from Acro Biosystems. Anti-6×his-peroxidase antibody (Sigma, 1:5000 dilution) was added to the plates and incubated for 1 hour at room temperature. After washing, the TMB substrate was added to the wells to develop color and the absorbance at 450 nm was measured using the Multiskan plate reader (Thermo). OD450 values were plotted against IL2RFβ concentration. As shown in FIG. 7, the anti-CTLA-4-IL-15Ra-sushi-IL-15 fusion protein can effectively interact with IL2Rβ.

### Example 5: Anti-CTLA-4-IL-15Ra-Sushi-IL-15 Fusion Proteins Promote Proliferation of T Cells

Anti-CTLA-4-IL-15Ra-sushi-IL-15 fusion proteins demonstrate strong activity in promoting the proliferation of both wild-type and IL15Ra-deficient T cells in vitro.

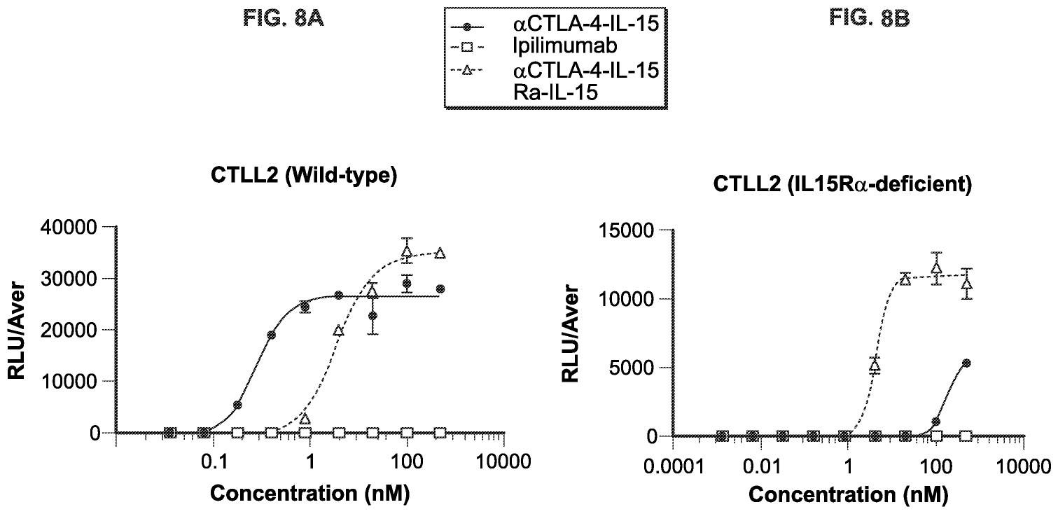

The activity of the anti-CTLA-4-IL-15Ra-sushi-IL-15 fusion protein (SEQ ID NOS: 9, 10 and 16) in promoting T cell proliferation was assessed using the CellTiter-Glo luminescent cell viability assay kit (Promega). Mouse T cell line CTLL-2 (wild-type) was obtained from ATCC, and IL15Ra-deficient CTLL-2 cells were generated in house. Both wild-type CTLL-2 and IL15Ra-deficient CTLL-2 cells were seeded in 96-well white-wall cell culture plates at 75 μL/well and a density of 1.33×10⁶ cells/mL. Anti-CTLA-4-IL-15Ra-sushi-IL-15 fusion protein, Anti-CTLA-4-IL-15 fusion protein (no sushi domain), and Ipilimumab, prepared in 5-fold serial dilutions (starting from 2 μM), were added to the plates at 25 μL per well. After 72 hours of incubation at 37° C. and 5% CO₂, the number of viable cells was assessed according to the manufacturer's instructions. Luminescence readout in each well was corrected by the background value from blank control wells and the ratio of luminescence readout (test to blank) was plotted against the antibody concentration. Unexpectedly, as shown in FIG. 8A, on the wild-type CTLL-2 cells, both the anti-CTLA-4-IL-15 fusion protein without sushi and the anti-CTLA4-IL-15Ra-sushi-IL-15 fusion protein were able to induce luciferase proliferation as measured by luminescence. Furthermore, the anti-CTLA-4-IL-15 fusion protein without the sushi domain exhibited a greater ability to induce proliferation than anti-CTLA4-IL-15Ra-sushi-IL-15. Also unexpectedly, as shown in FIG. 8B, when IL-15Ra was abrogated (by using IL15R(x-deficient CTLL-2 cells), the anti-CTLA4-IL-15Ra-sushi-IL-15 fusion protein retained the ability to induce proliferation, while the proliferation induced by the anti-CTLA-4-IL-15 fusion protein without the sushi domain was substantially attenuated.

Additional anti-CTLA-4-IL-15Ra-sushi-IL-15 fusion proteins with differing arrangements of the anti-CTLA-4 antibody, IL-15, IL-15Ra sushi domain, and linkers were also assayed for their ability to promote T cell proliferation using this assay. Diagrams of the constructs are provided in FIGS. 1B-1E, and the sequences are provided in Table 4 and Table 5.

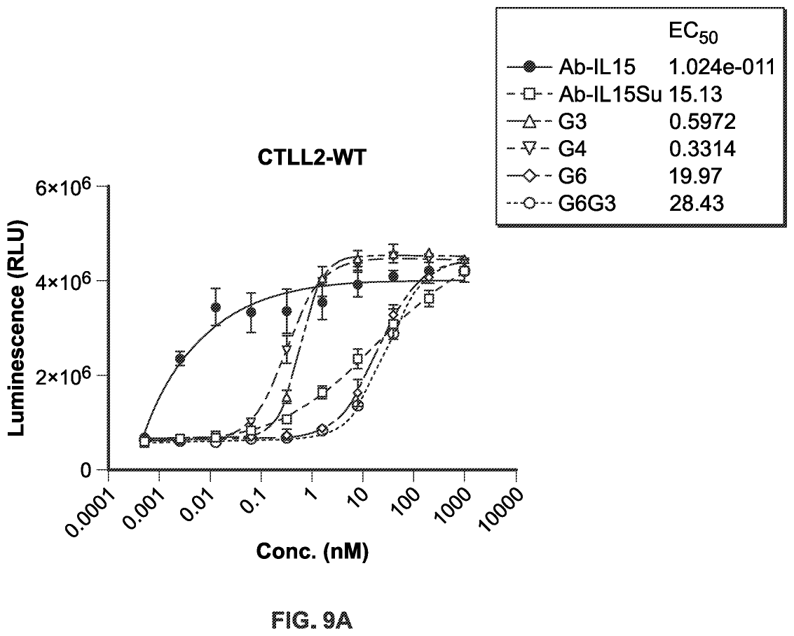

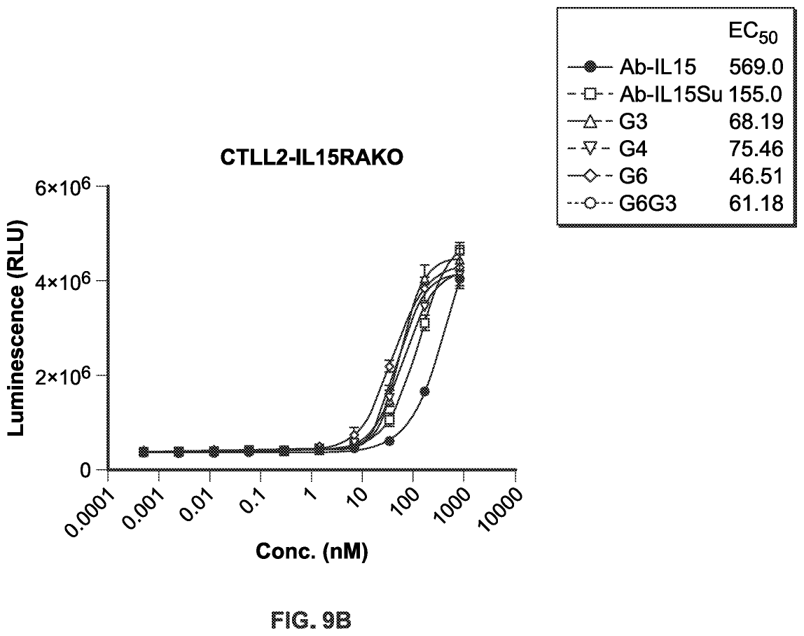

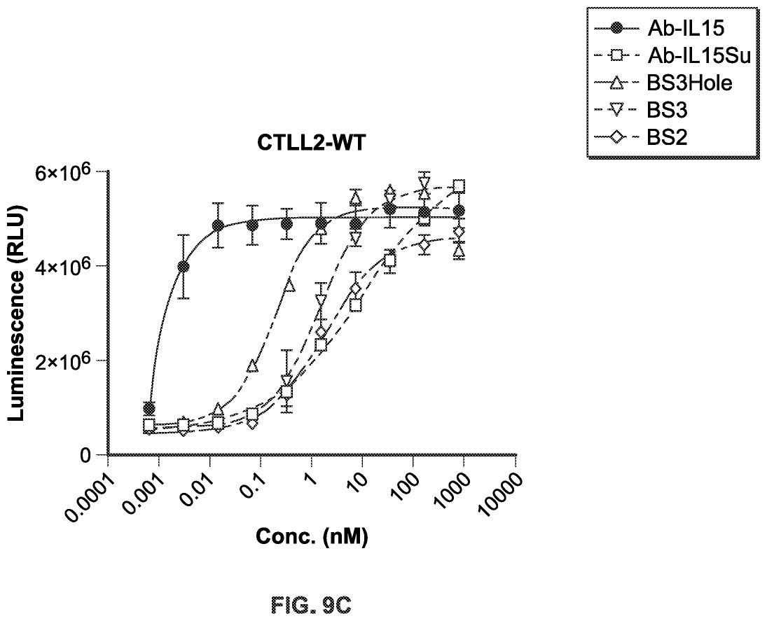

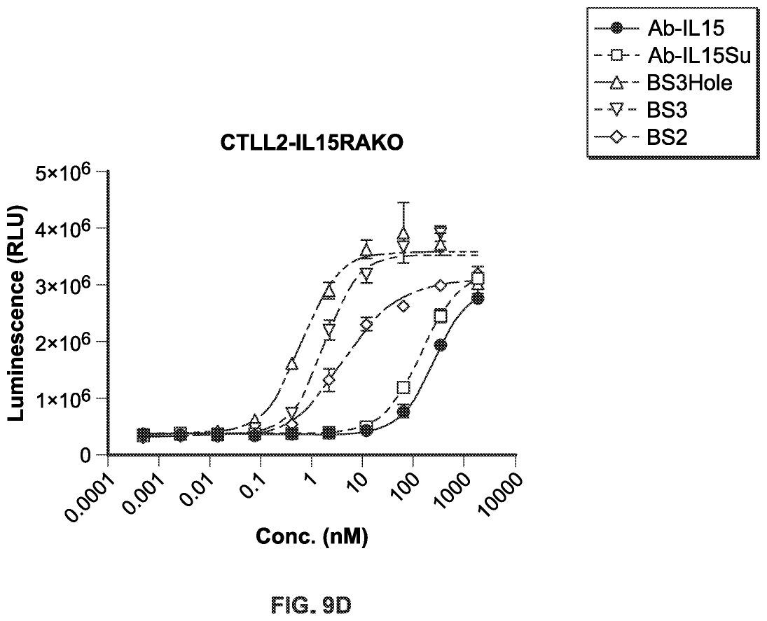

Results are shown in FIGS. 9A-9D. None of Ab-IL15 (sequences in Table 3), Ab-IL-15Su, G3, G4, G6 or G6G3 configurations showed proliferation in IL15Ra-deficient (CTLL2-IL15RAKO, FIG. 9B) T cells. Construct BS3 (sequences in Table 4) showed better activity than BS2 in inducing proliferation of CTLL2-IL15RAKO T cells (FIG. 9D).

TABLE 5
anti-CTLA-4-IL-15 and anti-CTLA-4-IL-15Ra-sushi-IL-15 construct heavy
chain sequences

| Name | Protein Sequence | DNA Sequence |
|---|---|---|
| Ab-IL15 (knob) | SEQ ID NO: 39 | SEQ ID NO: 38 |
| Ab-IL15 (hole) | SEQ ID NO: 41 | SEQ ID NO: 40 |
| Ab-IL15Su (knob) | SEQ ID NO: 39 | SEQ ID NO: 38 |
| Ab-IL15Su | SEQ ID NO: 44 | SEQ ID NO: 45 |
| (hole) | | |
| G2 (knob) | SEQ ID NO: 46 | SEQ ID NO: 47 |
| G2 (hole) | SEQ ID NO: 48 | SEQ ID ON: 49 |
| G3 (knob) | SEQ ID NO: 50 | SEQ ID NO: 51 |
| G3 (hole) | SEQ ID NO: 52 | SEQ ID NO: 53 |
| G4 (knob) | SEQ ID NO: 54 | SEQ ID NO: 55 |
| G4 (hole) | SEQ ID NO: 56 | SEQ ID NO: 57 |
| G6 (knob) | SEQ ID NO: 58 | SEQ ID NO: 59 |
| G6 (hole) | SEQ ID NO: 60 | SEQ ID NO: 61 |
| BS2 (knob) | SEQ ID NO: 62 | SEQ ID NO: 63 |
| BS2 (hole) | SEQ ID NO: 64 | SEQ ID NO: 65 |

Additional constructs use a light chain of SEQ ID NO: 9.

Ab-IL15: T366S, L368A, Y407T and Y350C from FIG. 1B refer to positions 367, 369,408 and 350 of SEQ ID NO: 41, respectively. L234F, S239A and N434A refer to positions 235, 240, and 435 of SEQ ID NO: 41, e.g. T366W and S354C refer to positions 367 and 355 of SEQ ID NO: 39.

Ab-IL15Su: T366S, L368A, Y407T and Y350C from FIG. 1B refer to positions 367, 369, 408 and 350 of SEQ ID NO: 44, respectively. L234F, S239A and N434A refer to positions 235, 240, and 435 of SEQ ID NO: 44, e.g. T366W and S354C refer to positions 367 and 355 of SEQ ID NO: 39.

G2: Ab-IL15: T366S, 1L368A, Y407V and Y350C from FIG. 1C refer to positions 367, 369, 408 and 350 of SEQ ID NO: 48, respectively. L234F, S239A and N434A refer to positions 235, 240, and 435 of SEQ ID NO: 48, e.g. T366W and S354C refer to positions 367 and 355 of SEQ ID NO: 46.

G3: T366W from FIG. 1D refers to position 366 of SEQ ID NO: 50. L234F, S239A and N434A refer to positions 235, 240, and 435 of SEQ ID NO: 52, e.g. Y407T refers to position 408 of SEQ ID NO: 52.

G4, G6 and G6G3 knob-hole mutations in FIGS. 1C-1D similarly refer to corresponding positions in the sequences provided in Table 5.

### Example 6: Anti-CTLA-4-IL-15Ra-Sushi-IL-1S Fusion Proteins Promote NK Cell and CD8⁺ T Cell Proliferation in C57BL/6 Mice

C57BL/6 mice (Gem Pharmatech, Nanjing, China) at 9-11 weeks old were randomized into 5 groups (15 animals/group), and designated each to receive one of the following treatments: PBS (vehicle control), anti-CTLA-4-IL-15 fusion protein (0.3 mg/kg), anti-CTLA-4-IL-15 fusion protein (1 mg/kg), anti-CTLA-4-IL-15Ra-sushi-IL-15 (0.3 mg/kg), and anti-CTLA-4-IL-15Ra-sushi-IL-15 (1 mg/kg). Mice were given a single-dose treatment through intraperitoneal injection. At 3, 5, and 7 days following the treatment, 5 animals from each group were euthanized and fresh blood and spleen cells were collected using conventional procedures. Each sample was stained with a mix of antibodies including PE-labeled anti-mouse CD3 (Biolegend), FITC-labeled anti-mouse CD4 (Biolegend), PE/Cy7-labeled anti-mouse CD8a (Biolegend), and APC-labeled anti-mouse CD335 (Biolegend). NK cells and CD8⁺ T cells in the blood and spleen samples were analyzed by flow cytometry and data were presented as the percent NK cells or CD8⁺ T cells in the total cell population (Mean±SEM, n=5). Statistical analysis of treatments in comparison to the vehicle control was conducted by one-way ANOVA plus paired t-test, and differences were considered significant if p<0.05.

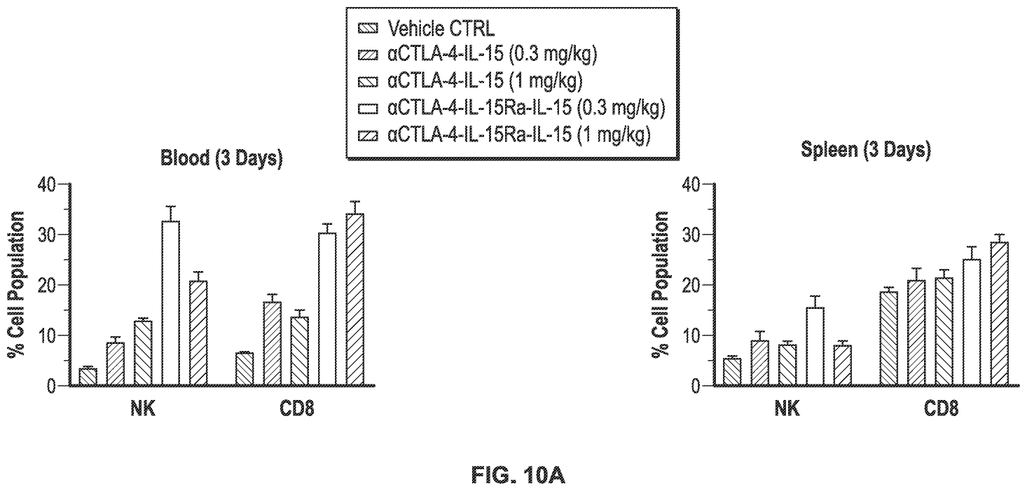

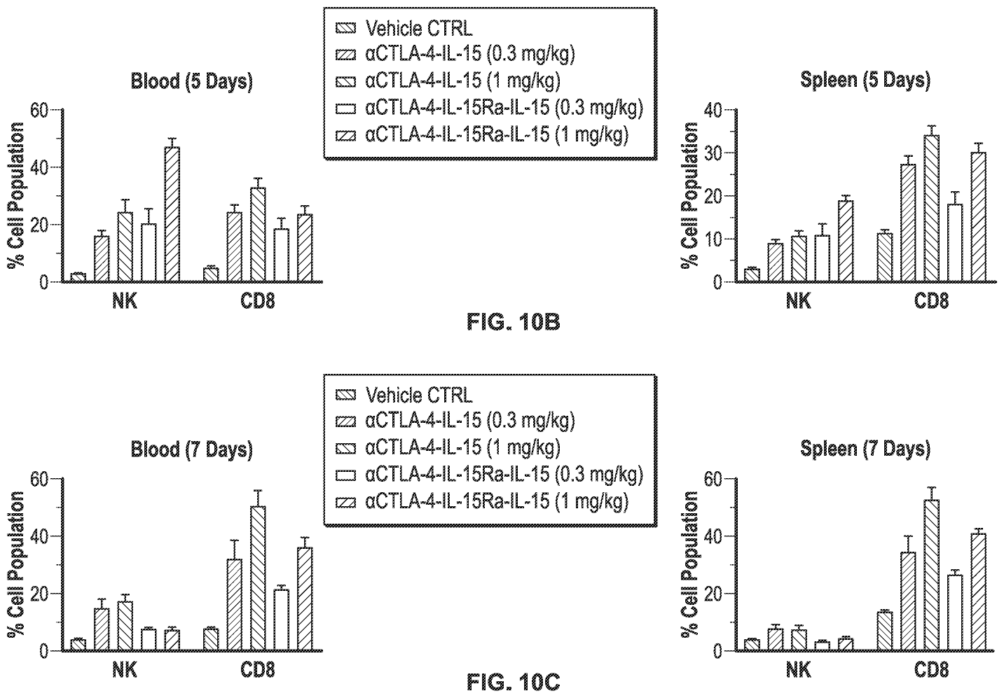

As shown in FIGS. 10A-10C, anti-CTLA-4-IL-15Ra-sushi-IL-15 at both doses (0.3 and 1 mg/kg) demonstrated strong activities in promoting NK cell and CD8⁺ T cell proliferation in the peripheral blood and the spleen of treated animals, although the activation response was more prominent in the peripheral blood. While the activity in stimulating CD8⁺ T cells appeared comparable between anti-CTLA-4 IL15 fusion proteins with and without the IL-15Ra sushi domain, the potency of the fusion protein with the IL-15Ra sushi domain in expanding NK cell population was much higher than that of the fusion protein without the IL-15Ra sushi domain.

Compared to the vehicle control, there was no significant body-weight loss in animals receiving treatments at 0.3 mg/kg dose. However a significant body-weight loss was observed in mice receiving either anti-CTLA-4-IL-15Ra-sushi-IL-15 or anti-CTLA-4-IL-15 at the 1 mg/kg dose. Nevertheless, the observed body-weight losses, if any, were all below 10% of the starting body weight throughout the study, suggesting that the treatment was well-tolerated by the animals.

### Example 7: Anti-CTLA-4-IL-15Ra-Sushi-IL-15 Fusion Protein Promotes T-Cell and NK Cell Proliferation in Cynomolgus Macaques

A total of 40 cynomolgus monkeys (5/sex per group), aged 2.5-4 years old and weighing 2.5-4 kg each, were randomly assigned into 4 groups (Groups 1-4). All groups were dosed once weekly for 4 consecutive weeks with Group 1 as the vehicle control group. Groups 2 and 3 received 0.4 and 0.8 mg/kg of anti-CTLA-4-IL-15Ra-sushi-IL-15 (SEQ ID NOS: 9, 10 and 16), respectively, via subcutaneous injection (s.c.). Group 4 received 1.6 mg/kg of anti-CTLA-4-IL-15Ra-sushi-IL-15 via subcutaneous injection for the first dose, and 1.0 mg/kg of JK08 via subcutaneous injection for the second, third, and fourth doses. At the timepoints of pre-dose, Day 2, Day 6, Day 23, and Day 27, approximately 1.0 mL blood was drawn into heparin sodium anticoagulant tubes for immunophenotyping analysis.

Samples were stored on ice and analyzed within two hours from the time the blood was drawn.

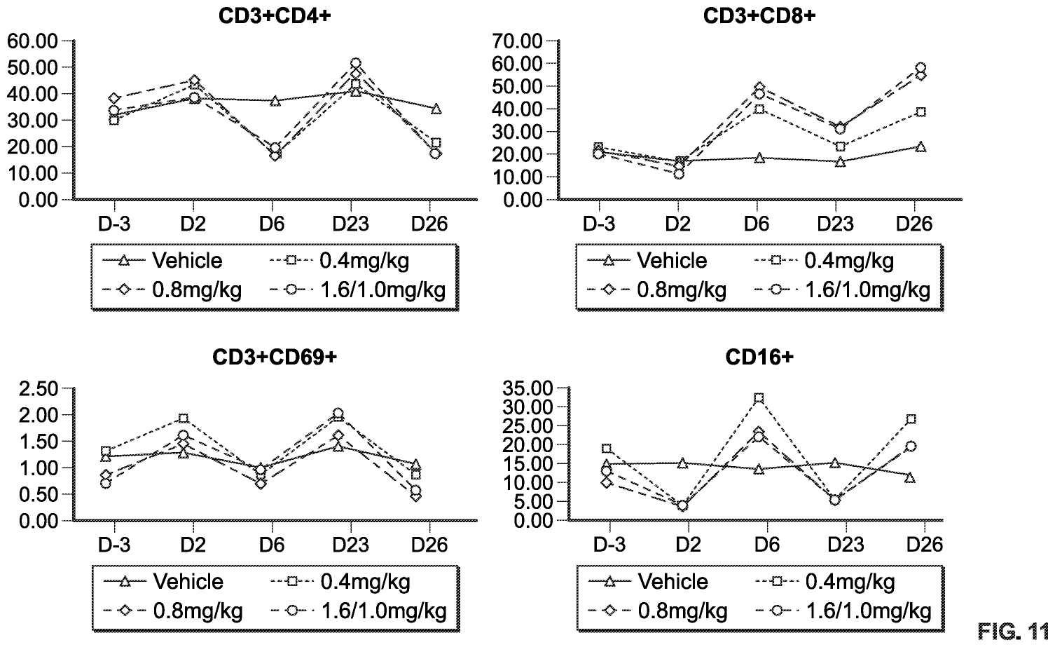

As shown in FIG. 11, anti-CTLA-4-IL-15Ra-sushi-IL-15 induced marked proliferation of CD16+ cells which represent the NK cell population, CD3+CD4+ T-cells, and CD3+CD8+ T-cells. Peripheral blood collection at the indicated time points was followed by standard antibody staining for CD16, CD3, CD4, CD8, and CD69, followed by FACS analysis.

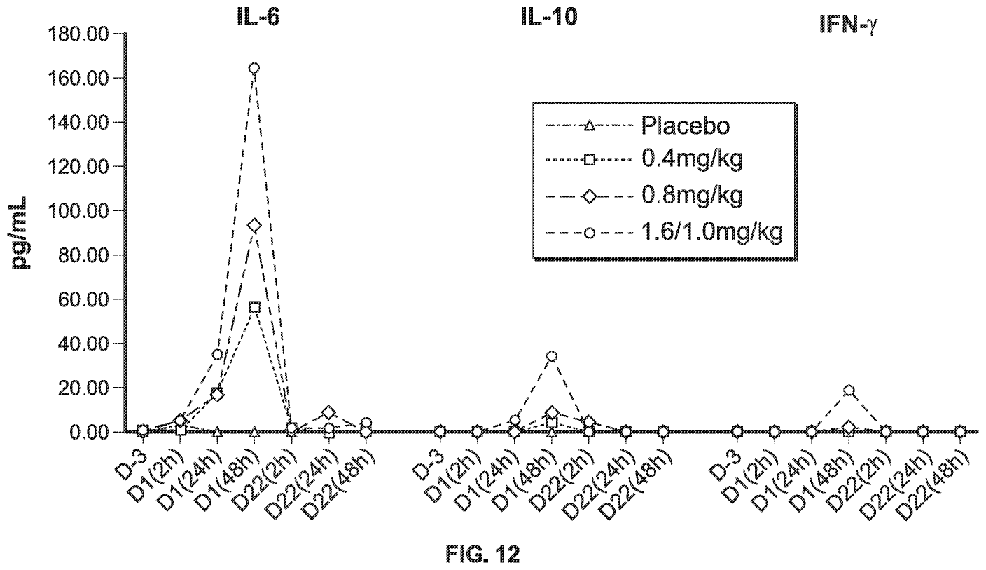

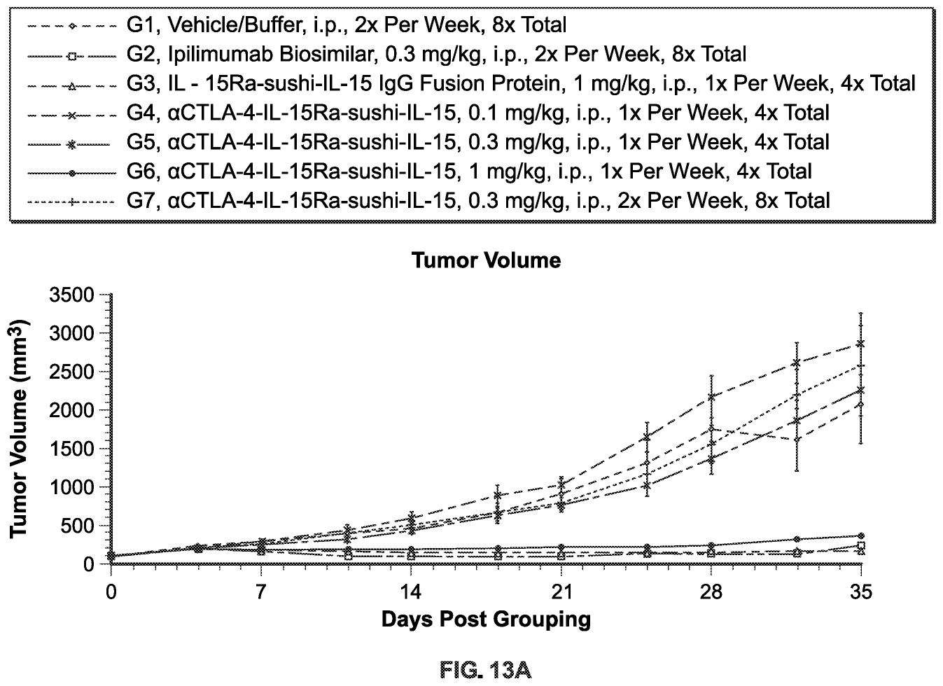

Levels of cytokines in peripheral blood were also assayed at the time points indicated in FIG. 12. Serum samples were isolated from whole blood samples and stored at ≤−65 degrees Celsius until analysis was performed. Analysis was performed with a validated electrochemiluminescence (MSD) method. As shown in FIG. 12, anti-CTLA-4-IL-15Ra-sushi-IL-15 induced cytokine expression detected in peripheral blood in a dose-dependent manner for IL-6 and IL-10. Unexpectedly, levels of IFNγ did not markedly increase with administration of the anti-CTLA-4-IL-15Ra-sushi-IL-15 fusion protein. This was particularly surprising given the observed increase in levels of IL-6 shown in FIG. 12. Furthermore, levels of TNFα, IL-2, 1L-4 and IL-8 did not increase, and remained largely below the limits of detection (not shown).

### Example 8: Anti-CTLA-4-IL-15Ra-Sushi-IL-15 Fusion Protein Show Anti-Cancer Activity in Mice Pressing Human CTLA-4

B-hCTLA4 mice (Biocytogen, Beijing, China) were subcutaneously injected with MC38 colon carcinoma tumor cells (5×10⁵ cells, Shunran Shanghai Biological Technology Co.) suspended in 0.1 mL PBS in the right front flank for tumor development. Tumor-bearing animals were randomly enrolled into seven study groups when the mean tumor size reaches 99 mm³. Each group consisted of 8 mice. The seven groups were: G1 Vehicle, G2 Ipilimumab (0.3 mg/kg), G3 IL15+Sushi Domain IgG Fusion Protein (1 mg/kg), G4 anti-CTLA-4-IL-15Ra-sushi-IL-15 (SEQ ID NOS: 9, 10 and 16) Low (0.1 mg/kg), G5 anti-CTLA-4-IL-15Ra-sushi-IL-15 Mid (0.3 mg/kg), G6 anti-CTLA-4-IL-15Ra-sushi-IL-15 High (1 mg/kg) and G7 anti-CTLA-4-IL-15Ra-sushi-IL-15 Mid (0.3 mg/kg). G1, G2 and G7 were intraperitoneally (i.p.) administrated to tumor-bearing mice at a frequency of twice per week for a total of eight administrations, and G3-G6 were intraperitoneally administrated to tumor-bearing mice at a frequency of once per week for a total of four administrations. All administrations were diluted in PBS to achieve the desired dose level at an appropriate volume for administration. Tumor volumes and body weights were measured and recorded twice per week. The study was terminated 36 days following the first dosing. At the end of this experiment, tumors were removed from euthanized animals, weighed and photographed.

B-hCTLA4 mice (Biocytogen, Beijing, China) were subcutaneously injected with B16F10 lung carcinoma tumor cells (1×10⁵)(ATCC) suspended in 0.1 mL PBS in the right front flank for tumor development. Tumor-bearing animals were randomly enrolled into seven study groups when the mean tumor size reaches 99 mm³. Each group consisted of 8 mice. The seven groups were G1 Vehicle, G2 Ipilimumab (5.0 mg/kg), G3 IL15+Sushi Domain IgG Fusion Protein (1 mg/kg), G4 anti-CTLA-4-IL-15Ra-sushi-IL-15 (SEQ ID NOS: 9, 10 and 16) Low (0.1 mg/kg), G5 anti-CTLA-4-IL-15Ra-sushi-IL-15 Mid (0.3 mg/kg), G6 anti-CTLA-4-IL-15Ra-sushi-IL-15 High (1 mg/kg) and G7 anti-CTLA-4-IL-15Ra-sushi-IL-15 Mid (0.3 mg/kg). G1, G2 and G7 were intraperitoneally administrated to tumor-bearing mice at a frequency of twice per week for a total of six administrations, and G3-G6 were intraperitoneally administrated to tumor-bearing mice at a frequency of once per week for a total of three administrations. All administrations were diluted in PBS to achieve the desired dose level at an appropriate volume for administration. Tumor volumes and body weights were measured and recorded twice per week. The study was terminated 18 days following the first dosing. At the end of this experiment, tumors were removed from euthanized animals, weighed and photographed.

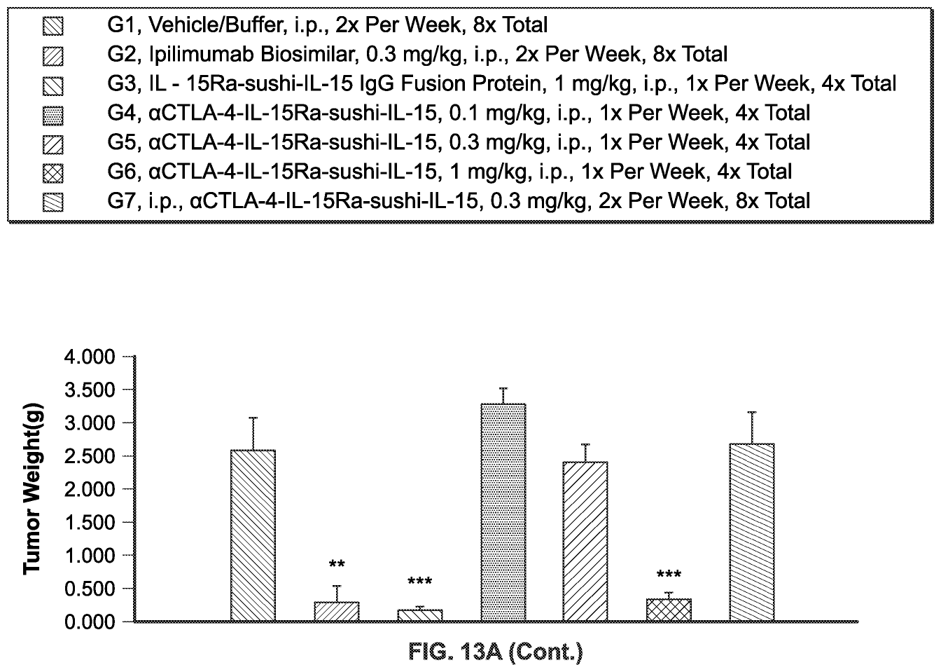

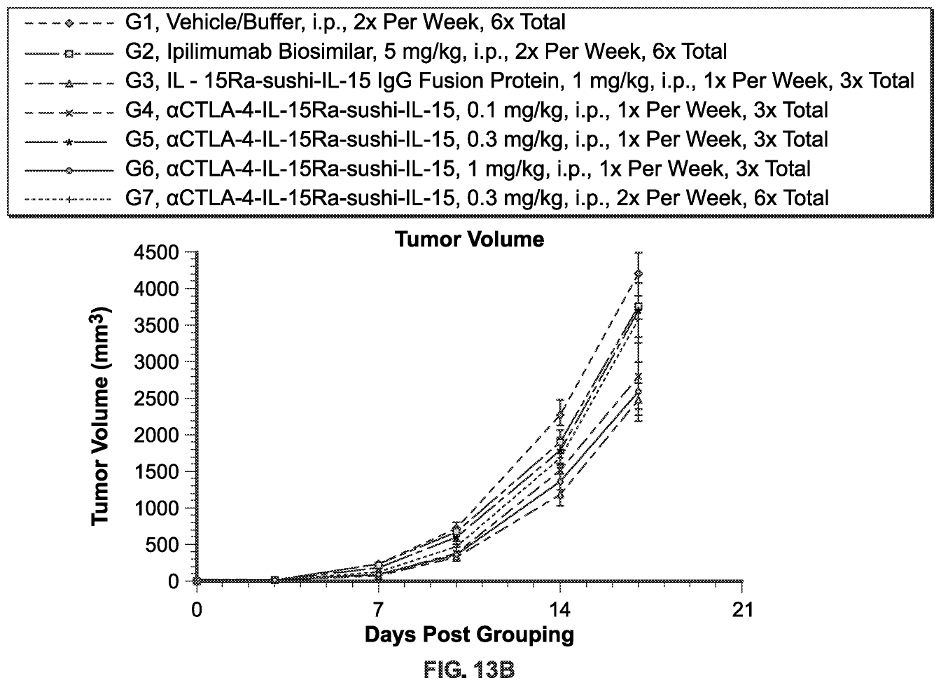

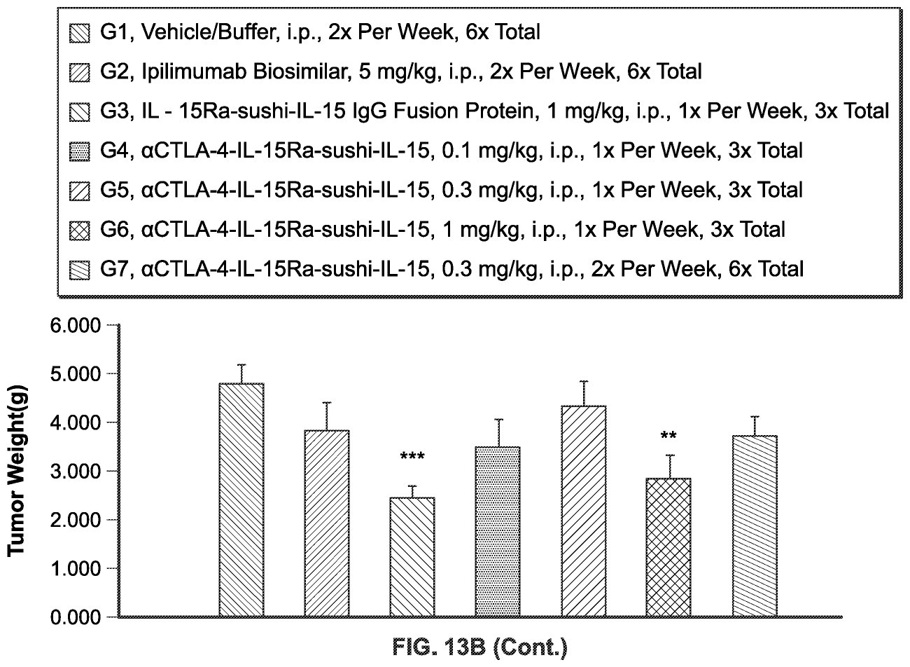

The results are shown in FIGS. 13A (MC38) and 13B (B16F10). As shown in FIG. 13A, administration of 1 mg/kg IL of anti-CTLA-4-IL-15Ra-sushi-IL-15 significantly reduced tumor volume and tumor weight in both MC38 mice (FIG. 13A) and B16F10 mice (FIG. 13B).

What is claimed is:

1. A recombinant fusion protein comprising:
a. an interleukin 15 (IL-15) domain;
b. an interleukin 15 receptor subunit alpha (IL-15Ra) sushi domain; and
c. a cytotoxic T-lymphocyte associated protein 4 (CTLA-4) antigen binding domain;
wherein the IL-15 domain and IL-15Ra sushi domain are separated by a GGGGSGGGGSGGGGSGGGGS (SEQ ID NO: 15) linker.

2. The recombinant fusion protein of claim 1, wherein the IL-15 domain is active.

3. The recombinant fusion protein of claim 1 or 2, wherein the IL-15Ra sushi domain increases the activity of the IL-15 domain compared to the activity of an IL-15 domain in an otherwise equivalent recombinant fusion protein lacking the IL-15Ra sushi domain.

4. The recombinant fusion protein of any one of claims 1-3, wherein the IL-15 domain comprises a sequence of NWVNVISDLKKIEDLIQSMHIDATLYTESDVHPSCKVTAMKCFLLELQVIS LESGDASIHDTVENLHLANNSLSSNGNVTESGCKECEELEEKNIKEFLQSF VHIVQMFINTS (SEQ ID NO: 1), or a sequence having at least 80%, at least 85%, at least 90%, at least 95% or at least 99°,% identity thereto.

5. The recombinant fusion protein of any one of claims 1-3, wherein the IL-15 domain comprises a sequence of SEQ ID NO: 1.

6. The recombinant fusion protein of any one of claims 1-5, wherein the IL-15Ra sushi domain comprises a sequence of ITCPPPMSVEHADIWVKSYSLYSRERYICNSGFKRKAGTSSLTECVLNKAT NVAHWTTPSLKCIRDPALVHQRPAPPSTV (SEQ ID NO: 2), or a sequence having at least 80%, at least 85%, at least 90%, at least 95% or at least 99% identity thereto.

7. The recombinant fusion protein of any one of claims 1-5, wherein the IL-15Ra sushi domain comprises a sequence of SEQ ID NO: 2.

8. The recombinant fusion protein of any one of claims 1-7, wherein the CTLA-4 antigen binding domain comprises a heavy chain comprising complementarity determining region (CDR) sequences of GFTFSSYT (SEQ ID NO: 5), ISYDGNNK (SEQ ID NO: 6) and ARTGWLGPFDY (SEQ ID NO: 7).

9. The recombinant fusion protein of any one of claims 1-8, wherein the CTLA-4 antigen binding domain comprises a light chain comprising CDR sequences of QSVGSSY (SEQ ID NO: 3), GAF and QQYGSSPWT (SEQ ID NO: 4).

10. The recombinant fusion protein of any one of claims 1-9, wherein the CTLA-4 antigen binding domain comprises a single chain variable fragment (scFv), a single-domain antibody (sdAb), an antibody, or an antibody fragment.

11. The recombinant fusion protein of any one of claims 1-9, wherein the CTLA-4 antigen binding domain comprises a CTLA-4 antibody.

12. The recombinant fusion protein of claim 11, wherein the CTLA-4 antibody comprises a first heavy chain and second heavy chain.

13. The recombinant fusion protein of claim 12, wherein the first and second heavy chains both comprise a heavy chain variable region sequence of QVQLVESGGGVVQPGRSLRLSCA ASGFTFSSYTMHWVRQAPGKGLEWV TFISYDGNNKYYADSVKGRFTISRDNSKNTLYLQMNSLRAEDTAIYYCAR TGWLGPFDYWGQGTLVTVSS (SEQ ID NO: 12), or a sequence having at least 80%, at least 85%, at least 90%, at least 95% or at least 99% identity thereto.

14. The recombinant fusion protein of claim 12, wherein the first and second heavy chains both comprise a heavy chain variable region sequence of SEQ ID NO: 12.

15. The recombinant fusion protein of any one of claims 12-14, wherein the first heavy chain comprises a constant region sequence of ASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKRVEPK SCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLSC AVKGFYPSDIAVEWESNGQPENNYKTTPPVLDSDGSFFLVSKLTVDKSRW QQGNVFSCSVMHEALHNHYTQKSLSLSPGK (SEQ ID NO: 13), or a sequence having at least 80%, at least 85%, at least 90%, at least 95% or at least 99% identity thereto.

16. The recombinant fusion protein of any one of claims 12-14, wherein the first heavy chain comprises a constant region sequence of SEQ ID NO: 13.

17. The recombinant fusion protein of any one of claims 12-16, wherein the second heavy chain comprises a constant region sequence of ASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKRVEPK SCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLW CLVKGFYPSDIAVEWESNGQPENNYKTTPPVLDSDGSFFLYSKLTVDKSR WQQGNVFSCSVMHEALHNHYTQKSLSLSPGK (SEQ ID NO: 14), or a sequence having at least 80%, at least 85%, at least 90%, at least 95% or at least 99% identity thereto.

18. The recombinant fusion protein of any one of claims 12-16, wherein the second heavy chain comprises a constant region sequence of SEQ TD NO: 14.

19. The recombinant fusion protein of any one of claims 12-16, wherein the first heavy chain comprises a sequence of QVQLVESGGGVVQPGRSLRLSCAASGFTFSSYTMHWVRQAPGKGLEWV TFISYDGNNKYYADSVKGRFTISRDNSKNTLYLQMNSLRAEDTAIYYCAR TGWLGPFDYWGQGTLVTVSSASTKGPSVFPLAPSSKSTSGGTAALGCLVK DYFPEPVTVSWNSGALTSGVHTFPAVLQSSGLYSLSSVVTVPSSSLGTQTY ICNVNHKPSNTKVDKRVEPKSCDKTHTCPPCPAPELLGGPSVFLFPPKPKD TLMISRTPEVTCVVVDVSHEDPEVKFNWYVDGVEVHNAKTKPREEQYNS TYRVVSVLTVLHQDWLNGKEYKCKVSNKALPAPIEKTISKAKGQPREPQ VYTLPPSREEMTKNQVSLSCAVKGFYPSDIAVEWESNGQPENNYKTTPPV LDSDGSFFLVSKLTVDKSRWQQGNVFSCSVMHEALHNHYTQKSLSLSPG K (SEQ ID NO: 11), or a sequence having at least 90%, at least 95% or at least 99% identity thereto.

20. The recombinant fusion protein of any one of claims 12-19, wherein the second heavy chain comprises a sequence of QVQLVESGGGVVQPGRSLRLSCAASGFTFSSYTMHWVRQAPGKGLEWV TFISYDGNNKYYADSVKGRFTISRDNSKNTLYLQMNSLRAEDTAIYYCAR TGWLGPFDYWGQGTLVTVSSASTKGPSVFPLAPSSKSTSGGTAALGCLVK DYFPEPVTVSWNSGALTSGVHTFPAVLQSSGLYSLSSVVTVPSSSLGTQTY ICNVNHKPSNTKVDKRVEPKSCDKTHTCPPCPAPELLGGPSVFLFPPKPKD TLMISRTPEVTCVVVDVSHEDPEVKFNWYVDGVEVHNAKTKPREEQYNS TYRVVSVLTVLHQDWLNGKEYKCKVSNKALPAPIEKTISKAKGQPREPQ VYTLPPSREEMTKNQVSLWCLVKGFYPSDIAVEWESNGQPENNYKTTPP VLDSDGSFFLYSKLTVDKSRWQQGNVFSCSVMHEALHNHYTQKSLSLSP GK (SEQ ID NO: 10), or a sequence having at least 80%, at least 85%, at least 90%, at least 95% or at least 99% identity thereto.

21. The recombinant fusion protein of any one of claims 12-20, wherein the first and second heavy chains preferentially form a heterodimer.

22. The recombinant fusion protein of any one of claims 12-21, wherein the N-terminus of the IL-15Ra sushi domain is linked to the C-terminus of the first or second heavy chain.

23. The recombinant fusion protein of any one of claims 1-22, wherein the N-terminus of IL-15 domain is linked to the C-terminus of the IL-15Ra sushi domain.

24. The recombinant fusion protein of claim 22 or 23, wherein the first or second heavy chain and the IL-15Ra domain are separated by a linker.

25. The recombinant fusion protein of claim 24, wherein the linker comprises a sequence of GGGS (SEQ ID NO. 23), GGGGS (SEQ ID NO: 24), GGGGSGGGGS (SEQ ID NO: 25), GGGGSGGGGSGGGGS (SEQ ID NO: 26), or GGGGSGGGGSGGGGSGGGGS (SEQ ID NO: 15).

26. The recombinant fusion protein of any one of claims 22-25, wherein the first heavy chain, IL-15Ra sushi domain and IL-15 domain comprise a sequence of QVQLVESGGGVVQPGRSLRLSCAASGFTFSSYTMHWVRQAPGKGLEWV TFISYDGNNKYYADSVKGRFTISRDNSKNTLYLQMNSLRAEDTAIYYCAR TGWLGPFDYWGQGTLVTVSSASTKGPSVFPLAPSSKSTSGGTAALGCLVK DYFPEPVTVSWNSGALTSGVHTFPAVLQSSGLYSLSSVVTVPSSSLGTQTY ICNVNHKPSNTKVDKRVEPKSCDKTHTCPPCPAPELLGGPSVFLFPPKPKD TLMISRTPEVTCVVVDVSHEDPEVKFNWYVDGVEVHNAKTKPREEQYNS TYRVVSVLTVLHQDWLNGKEYKCKVSNKALPAPIEKTISKAKGQPREPQ VYTLPPSREEMTKNQVSLSCAVKGFYPSDIAVEWESNGQPENNYKTTPPV LDSDGSFFLVSKLTVDKSRWQQGNVFSCSVMHEALHNHYTQKSLSLSPG KGGGGGSGGGGSGGGGSITCPPPMSVEHADIWVKSYSLYSRERYICNSGF KRKAGTSSLTECVLNKATNVAHWTTPSLKCIRDPALVHQRPAPPSTVGGG GSGGGGSGGGGSGGGGSNWVNVISDLKKIEDLIQSMHIDATLYTESDVHP SCKVTAMKCFLLELQVISLESGDASIHDTVENLIILANNSLSSNGNVTESGC KECEELEEKNIKEFLQSFVHIVQMFINTS (SEQ ID NO: 16), or a sequence having at least 80%, at least 85%, at least 90%, at least 95% or at least 99% identity thereto.

27. The recombinant fusion protein of any one of claims 11-26, wherein the CTLA-4 antibody comprises a light chain sequence comprising EIVLTQSPGTLSLSPGERATLSCRA SQSVGSSYLAWYQQKPGQAPRLLIYG AFSRA TGIPDRFSGSGSGTDFTLTISRLEPEDFAVYYCQQYGSSPWTFGQG TKVEIKRTVAAPSVFIFPPSDEQLKSGTA SVVCLLNNFYPREAKVQWKVD NALQSGNSQESVTEQDSKDSTYSLSSTLTLSKADYEKHKVYACEVTHQG LSSPVTKSFNRGEC (SEQ ID NO: 9), or a sequence having at least 80%, at least 85%, at least 90%, at least 95% or at least 99% identity thereto.

28. The recombinant fusion protein of any one of claims 11-26, wherein the CTLA-4 antibody comprises a light chain sequence comprising SEQ ID NO: 9.

29. A recombinant fusion protein, comprising:
a. a first polypeptide comprising, from N- to C-terminus, sequences of a first CTLA-4 antibody heavy chain, an IL-15Ra sushi domain and an IL-15 domain, wherein the IL-15 domain and IL-15Ra sushi domain are linked using a GGGGSGGGGSGGGGSGGGGS (SEQ ID NO: 15) linker;
b. a second polypeptide comprising a sequence of a second CTLA-4 heavy chain; and
c. two additional polypeptides comprising a sequence of a CTLA-4 antibody light chain.

30. The recombinant fusion protein of claim 29, wherein the first and second polypeptides preferentially form a heterodimer.

31. The recombinant fusion protein of claim 29 or 30, wherein the first polypeptide comprises a sequence of SEQ ID NO: 16, the second polypeptide comprises a sequence of SEQ ID NO: 10, and the CTLA-4 antibody light chain comprises a sequence of SEQ ID NO: 9, or a sequence having at least 80%/6, at least 85%, at least 90%, at least 95% or at least 99% identity thereto.

32. A polynucleotide encoding the recombinant fusion protein of any one of claims 1-28.

33. A polynucleotide encoding the first polypeptide, second polypeptide, or the CLTA-4 antibody light chain of any one of claims 29-32.

34. The polynucleotide of claim 33, wherein the sequence encoding the CTLA-4 antibody light chain comprises a sequence of SEQ ID NO: 17, or a sequence having at least 80%, at least 85%, at least 90%, at least 95% or at least 99% identity thereto.

35. The polynucleotide of claim 33, wherein the sequence encoding the first polypeptide comprises a sequence of SEQ ID NO: 18, or a sequence having at 80%, at least 85%, at least 90%, at least 95% or at least 99% identity thereto.

36. The polynucleotide of claim 33, wherein the sequence encoding the second polypeptide comprises a sequence of SEQ ID NO: 19 or a sequence having at least 80%, at least 85%, at least 90%, at least 95% or at least 99% identity thereto.

37. A vector comprising the polynucleotide of any one of claims 32-36.

38. The vector of claim 37, further comprising a promoter operably linked to the sequence encoding the recombinant fusion protein or polynucleotide.

39. A pharmaceutical composition comprising the recombinant fusion protein of any one of claims 1-31 and a pharmaceutically acceptable carrier, diluent or excipient.

40. The pharmaceutical composition of claim 39, wherein the pharmaceutical composition is suitable for parenteral administration.

41. The pharmaceutical composition of claim 39 or 40, wherein the parenteral administration comprises intravenous infusion or injection, intratumoral injection, or subcutaneous injection.

42. A method of treating a subject with a disease or disorder, comprising administering a therapeutically effective amount of the recombinant fusion protein of any one of claims 1-31 or the pharmaceutical composition of any one of claims 39-41.

43. The method of claim 42, wherein the disease or disorder is cancer.

44. The method of claim 43, wherein the cancer comprises a solid tumor or a liquid tumor.

45. The method of claim 44, wherein the liquid tumor comprises leukemia, acute myeloid leukemia, myeloma, acute myelogenous leukemia (AML), acute lymphoblastic leukemia (ALL), lymphoma, Hodgkin's lymphoma, non-Hodgkin lymphoma, beta-cell lymphoma, chronic lymphocytic leukemia, chronic myelogenous leukemia, mantle cell lymphoma, follicular lymphoma, T-cell lymphoma, NK-cell lymphoma, B-cell lymphoma or NKT-cell lymphoma.

46. The method of claim 43, wherein the cancer is selected from the group consisting of melanoma, renal cell carcinoma, mesothelioma, small cell lung cancer, uveal melanoma, bladder cancer, gastric cancer, squamous cell carcinoma of the head and neck, cutaneous carcinoma, non-small cell lung cancer, colorectal cancer, prostate cancer, ovarian cancer, cervical cancer, endometrial carcinoma, breast cancer, pancreatic cancer, urothelial cancer, hepatocellular carcinoma, esophageal cancer, glioblastoma, glioma, or sarcoma.

47. The method of claim 43, wherein the cancer is selected from the group consisting of melanoma, and renal cell carcinoma.

48. The method of any one of claims 42-47, wherein the recombinant fusion protein or pharmaceutical composition inhibits the activity of CTLA-4 on an immune cell.

49. The method of any one of claims 42-48, wherein the recombinant fusion protein or pharmaceutical composition increases the activity of an Interleukin 2/Interleukin 15 receptor beta (IL-2Rb)/common gamma chain (IL-2RG) receptor complex on an immune cell.

50. The method of any one of claims 42-49, wherein the recombinant fusion protein or pharmaceutical composition promotes an activity in an immune cell.

51. The method of claim 50, wherein the activity comprises activation, proliferation, or a combination thereof.

52. The method of any one of claims 48-51, wherein the immune cell is a T cell, B cell or an NK cell.

53. The method of claim 52, wherein the T cell is a CD8+ T cell.

54. The method of any one of claims 42-53, wherein the recombinant fusion protein or pharmaceutical composition increases proliferation of NK cells.

55. The method of any one of claims 42-54, wherein the recombinant fusion protein or pharmaceutical composition is administered parenterally.

56. The method of claim 55, wherein the parenteral administration comprises intravenous infusion or injection, intratumoral injection, or subcutaneous injection.

57. The method of any one of claims 43-56, wherein administration of the recombinant fusion protein or pharmaceutical composition alleviates a sign or a symptom of the cancer.

58. The method of any one of claims 43-56, wherein administration of the recombinant fusion protein or pharmaceutical composition inhibits the progression of the cancer.

59. The method of any one of claims 43-58, further comprising one or more additional cancer therapies.

60. The method of claim 59, wherein the one or more additional cancer therapies comprises a chemotherapy, a small molecule inhibitor, a protein or biologic based therapy, radiation, surgery, immunotherapy or adoptive cell therapy.

61. The method of claim 60, wherein the adoptive cell therapy comprises a chimeric antigen receptor (CAR) T cell therapy, a T Cell Receptor (TCR) T cell therapy or a CAR NK cell therapy.

62. The method of any one of claims 42-61, wherein administration of the recombinant fusion protein or pharmaceutical composition does not substantially increase a level of interferon gamma (IFNγ) in a peripheral blood sample from the subject.

63. The method of any one of claims 42-61, wherein administration of the recombinant fusion protein or pharmaceutical composition increases proliferation of immune cells, but does not substantially increase a level of IFNγ in the subject.

64. The method of claim 63, wherein the immune cells comprise NK cells, CD8+ T cells, or a combination thereof.

65. The method of any one of claims 42-64, wherein administration of the recombinant fusion protein or pharmaceutical composition increases a level of interferon gamma (IFNγ) in a peripheral blood sample from the subject less than administration of an equimolar amount of IL-15 or IL-15 in a complex with the IL-15Ra sushi domain.

66. The method of any one of claims 42-65, administration of the recombinant fusion protein or pharmaceutical composition results in less toxicity than administration of an equimolar amount of IL-15 or TL-15 in a complex with the IL-15Ra sushi domain.

67. The method of any one of claims 42-66, wherein administration of the recombinant fusion protein or pharmaceutical composition results in a ratio of IL-6 to IFNγ that is greater than or equal to 2:1, 3:1, 4:1, 5:1, 6:1, 7:1, 8:1, 9:1 or 10:1.

68. The method of any one of claims 42-67, wherein the recombinant fusion protein is administered at a dose of 0.1 μg/kg to 1 mg/kg.

69. The method of any one of claims 42-67, wherein the recombinant fusion protein is administered at a dose of 10 μg/kg to 0.30 mg/kg.

70. The method of any one of claims 42-69, wherein the recombinant fusion protein or pharmaceutical composition is administered intravenously, intratumorally or subcutaneously.

71. The method of any one of claims 42-70, wherein the recombinant fusion protein or pharmaceutical composition is administered daily, every 2 days, every 3 days, every 4 days, every 5 days, every 6 days, every 7 days, every 8 days, every 9 days, every 10 days, every two weeks, every three weeks or monthly.

72. The method of any one of claims 42-71, wherein the recombinant fusion protein or pharmaceutical composition is administered for at least one week, at least two weeks, at least three weeks, at least 4 weeks, at least 5 weeks, at least 6 weeks, at least 2 months, at least 3 months, at least 4 months, at least 5 months, at least 6 months, at least 8 months, at least 10 months, at least 12 months, at least 14 months, at least 16 months, at least 18 months, at least 20 months, at least 22 months or at least 2 years.

73. The recombinant fusion protein of any one of claims 1-31 or the pharmaceutical composition of any one of claims 39-41, for use in a method of treating of a disease or disorder in a subject.

74. The recombinant fusion protein of any one of claims 1-31, for use the manufacture of a medicament for treating a disease or disorder in a subject.

75. A method of making the recombinant fusion protein of any one of claims 1-31, comprising:
a. contacting a plurality of cells with the polynucleotide of any one of claims 32-36 or the vector of claim 37 or 38;
b. expressing the recombinant fusion protein by the plurality of cells; and
c. purifying the recombinant fusion protein.

76. A kit, comprising a therapeutically effective amount of the recombinant fusion protein of any one of claims 1-31, the polynucleotide of any one of claims 32-36, the vector of claim 37 or 38, or the pharmaceutical composition of any one of claims 39-41.
